SEQUENCE LISTING (27 sequences)

SEQ ID NO: 1 - DNA, 25 bp, synthetic construct
  source 1..25: mol_type = other DNA
ccacgcgtggaacccttgggatcca

SEQ ID NO: 2 - protein, 10 aa, Synthetic construct
DDGGGDDDFF

SEQ ID NO: 3 - protein, 10 aa, Synthetic construct
GPPPPPPPPG

SEQ ID NO: 4 - protein, 5 aa, Synthetic construct
  MOD_RES 1: note = Isoglutamic acid
EGWRW

SEQ ID NO: 5 - protein, 14 aa, Synthetic construct
GGSSSGSGNDEEFQ

SEQ ID NO: 6 - protein, 12 aa, Synthetic construct
GGGGGDPDPDFF

SEQ ID NO: 7 - protein, 12 aa, Synthetic construct
GDGDGDGDGDFF

SEQ ID NO: 8 - protein, 10 aa, Synthetic construct
NNGGGNNNFF

SEQ ID NO: 9 - protein, 10 aa, Synthetic construct
  MOD_RES 6..8: note = X is a cysteic acid
DDGGGXXXFF

SEQ ID NO: 10 - protein, 15 aa, Synthetic construct
EFIAWLVAYPDDDWK

SEQ ID NO: 11 - protein, 18 aa, Synthetic construct
ALYKKLLKKLLKSAKKLG

SEQ ID NO: 12 - protein, 13 aa, Synthetic construct
RLYRKRIWRSAGR

SEQ ID NO: 13 - protein, 11 aa, Synthetic construct
LDYVNRRKMYQ

SEQ ID NO: 14 - protein, 17 aa, Synthetic construct
EMRISRIILDFLFLRKK

SEQ ID NO: 15 - protein, 23 aa, Synthetic construct
RIIDLLWRVRRPQKPKFVTVWVR

SEQ ID NO: 16 - protein, 10 aa, Synthetic construct
ASVYAWNRKR

SEQ ID NO: 17 - protein, 13 aa, Synthetic construct
FVQWFSKFLGRIL

SEQ ID NO: 18 - protein, 15 aa, Synthetic construct
WWGKKYRASKLGLAR

SEQ ID NO: 19 - protein, 10 aa, Synthetic construct
TFLLRNPNDK

SEQ ID NO: 20 - protein, 9 aa, Synthetic construct
ILRGSVAHK

SEQ ID NO: 21 - protein, 15 aa, Synthetic construct
  MOD_RES 4: note = Phosphoserine
RSRSGEGEVSGLMRK

SEQ ID NO: 22 - protein, 31 aa, Synthetic construct
  MOD_RES 20: note = Phosphoserine
FSAVLVEPPPMSLPGAGLSSQELSGGPGDGP

SEQ ID NO: 23 - protein, 24 aa, Synthetic construct
  MOD_RES 13: note = Phosphoserine
VQYLTRSAIRRASTIEMPQQARQK

SEQ ID NO: 24 - protein, 10 aa, Synthetic construct
ETFSDLWKLL

SEQ ID NO: 25 - protein, 30 aa, Synthetic construct
MEKVQYLTRSAIRRASTIEMPQQARQKLQN

SEQ ID NO: 26 - protein, 24 aa, Synthetic construct
VQYLTRSAIRRACTIEMPQQARQK

SEQ ID NO: 27 - DNA, 25 bp, synthetic construct
  source 1..25: mol_type = other DNA
tggagtcaaggtcctctgatgccat

(19) United States
(12) Patent Application Publication
(10) Pub. No.: US 2024/0182950 A1
(43) Pub. Date: Jun. 6, 2024

(54) METHODS AND SYSTEMS FOR CHARACTERIZING PHOSPHOSERINE-CONTAINING POLYPEPTIDES

(71) Applicant: Quantum-Si Incorporated, Branford, CT (US)

(72) Inventors: David N. Kamber, Guilford, CT (US); Kenneth Skinner, Cambridge, MA (US); Haidong Huang, Madison, CT (US)

(73) Assignee: Quantum-Si Incorporated, Branford, CT (US)

(21) Appl. No.: 18/491,699

(22) Filed: Oct. 20, 2023

Related U.S. Application Data
(60) Provisional application No. 63/418,295, filed on Oct. 21, 2022.

(51) Int. Cl. C12Q 1/6806; C12Q 1/6834

(52) U.S. Cl. CPC C12Q 1/6806; C12Q 1/6834

(57) ABSTRACT

According to some aspects, systems and methods for characterizing phosphoserine-containing polypeptides are described. In some embodiments, a phosphoserine residue of a polypeptide may be converted to a dehydroalanine residue. Some aspects are directed to a method of reacting a polypeptide comprising at least one dehydroalanine residue with a protected thiol compound comprising a click chemistry handle (e.g., an azide, an alkyne) to provide a functionalized polypeptide comprising a click chemistry handle. Some aspects are directed to systems and methods of characterizing phosphoserine residues in polypeptides (e.g., identifying and/or determining the location of a phosphoserine residue) and/or methods of preparing samples for such systems and methods.

### CROSS-REFERENCE TO RELATED APPLICATIONS

The present application claims the benefit of priority of US Provisional Application No. 63/418,295 filed Oct. 21, 2022, the entire content of which is incorporated herein by reference.

### REFERENCE TO AN ELECTRONIC SEQUENCE LISTING

The contents of the electronic sequence listing (R070870171US01-SEQ-DFC.xml; Size: 26,482 bytes; and Date of Creation: Oct. 20, 2023) is herein incorporated by reference in its entirety.

### FIELD

Methods and systems for characterizing phosphoserine-containing polypeptides are generally described.

### BACKGROUND

Proteins in biological systems often undergo post-translational modifications (PTMs) as strategic responses that mediate diverse cell regulatory processes, including protein conformation, activity, activity, stability, interaction profiles, and trafficking. Thus, PTMs vastly augment the functional diversity of the proteome.

While irreversible PTMs (e.g., citrullination) are often markers of aging or tissue injury, reversible PTMs (e.g., phosphorylation) often operate on rapid time scales that reflect a particular cellular state. Indeed, more than 500 human kinases, which catalyze phosphorylation, and more than 150 phosphatases, which catalyze dephosphorylation, regulate most cellular functions. Protein kinases are important in health and disease as they not only stimulate cell growth and proliferation but also other potential oncogenic properties. In addition, more than 100,000 different phosphorylation sites in the human proteome have been reported. Accordingly, kinases are attractive clinical drug targets, and localizing phosphorylated sites is key to elucidating cellular dysregulation and signal transmission. However, several factors limit the facile mapping of phosphorylated sites, which is key to a mechanistic understanding of the functional diversity of the proteome.

For one, phosphorylation is often transient as phosphatases can readily remove phosphate groups. Thus, to identify phosphorylated sites, covalent capture methods are often employed. For example, the labile nature of the phosphoserine group can be exploited to form a conjugation handle and couple with chemical probes that form covalent bonds. Immunoblotting and mass spectrometry (MS) are two of the most widely used techniques for phosphorylation detection. However, raising antibodies specific to phosphorylated and non-phosphorylated proteins is laborious. And while MS offers a highly sensitive approach to phosphopeptide localization, fragmentation (MS/MS) often leads to release of the phosphate moiety with concomitant breakage of the peptide backbone. This drawback can lead to ambiguities in phosphorylation sites. MS can also present discrepancies in assigning phosphorylation to histidine or neighboring serine/threonine residues. Moreover, assigning phosphorylation sites to peptides with the same PTM on different residues is another common problem. These isobaric (identical mass) phosphopeptides with equivalent sequences will elute closely and co-fragment, which presents hurdles for localization software to assign site probabilities.

In addition to difficulties in pinpointing phosphorylation sites, the substoichiometric nature of phosphorylation hampers automated MS/MS analysis as unmodified precursor ions will be biased and automatically selected for MS/MS rather than phosphopeptides with scant abundance.

Accordingly, improved methods and systems for characterizing phosphoserine-containing polypeptides are needed.

### SUMMARY

Methods and systems for characterizing phosphoserine-containing polypeptides are generally described.

In some aspects, a method is provided. In some embodiments, the method comprises reacting a polypeptide comprising at least one dehydroalanine residue with a protected thiol compound of Formula (I):

R₁-(L₁)ₘ-S—X (I),

or a salt thereof, to provide a functionalized polypeptide comprising a residue of Formula (II):

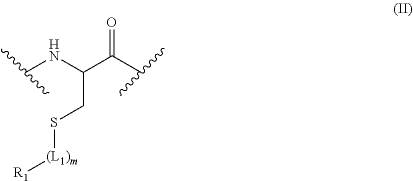

wherein:
X is a protecting group;
L₁ is a linking group;
m is an integer from 0 to 36; and
R₁ is a moiety comprising a click chemistry handle.

In some aspects, a method is provided. In some embodiments, the method comprises converting a phosphoserine residue of a polypeptide to a dehydroalanine residue to provide a dehydroalanine-containing polypeptide. In some embodiments, the method comprises reacting the dehydroalanine-containing polypeptide with a protected thiol compound of Formula (I):

R₁-(L₁)ₘ-S—X (I),

or a salt thereof, to provide a functionalized polypeptide comprising a residue of Formula (II):

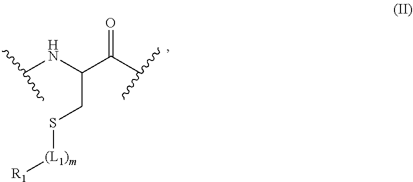

wherein:
X is a protecting group
L₁ is a linking group;
m is an integer from 0 to 36; and
R₁ is a moiety comprising a click chemistry handle.

In some embodiments, the method comprises digesting the functionalized polypeptide to form two or more peptide fragments, wherein at least one peptide fragment is a functionalized peptide fragment comprising R₁. In some embodiments, the method comprises conjugating the functionalized peptide fragment to a linker.

In some aspects, a method is provided. In some embodiments, the method comprises digesting a polypeptide comprising a phosphoserine residue to form two or more peptide fragments, wherein at least one peptide fragment comprises the phosphoserine residue. In some embodiments, the method comprises converting the phosphoserine residue of the at least one peptide fragment to a dehydroalanine residue of the at least one peptide fragment to provide a dehydro alanine-containing peptide fragment. In some embodiments, the method comprises reacting the dehydroalanine-containing peptide fragment with a protected thiol compound of Formula (I):

R₁-(L₁)ₘ-S—X (I),

or a salt thereof, to provide a functionalized peptide fragment comprising a residue of Formula (II):

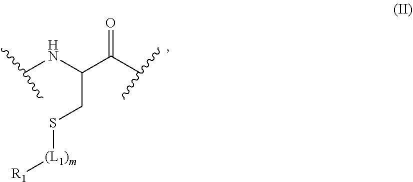

wherein:
X is a protecting group;
L₁ is a linking group;
m is an integer from 0 to 36; and
R₁ is a moiety comprising a click chemistry handle;

In some embodiments, the method comprises conjugating the functionalized peptide fragment to a linker.

In some aspects, a kit is provided. In some embodiments, the kit comprises a protected thiol compound comprising a click chemistry handle. In some embodiments, the kit comprises a base. In some embodiments, the kit comprises a linker.

In some aspects, a method of enriching a sample comprising one or more polypeptides comprising a target residue is provided. In some embodiments, the method comprises selectively conjugating the target residue to a linker. In some embodiments, the method comprises immobilizing the linker to a surface. In some embodiments, the method comprises removing any non-immobilized polypeptides or portions of polypeptides.

In some aspects, a system is provided. In some embodiments, the system comprises an integrated device comprising a sample well. In some embodiments, the system comprises a peptide immobilized to a surface of the sample well at a target residue. In certain embodiments, the target residue is not a terminal amino acid of the peptide.

In some aspects, a method of characterizing a peptide is provided. In some embodiments, the method comprises immobilizing the peptide to a surface of a sample well at a target residue, wherein the target residue is not a terminal amino acid of the peptide. In some embodiments, the method comprises contacting the peptide with one or more amino acid recognition molecules labeled with one or more detectable labels. In some embodiments, the method comprises detecting one or more series of signal pulses indicative of binding events between the one or more amino acid recognition molecules and the peptide. In some embodiments, the method comprises determining at least one chemical characteristic of the peptide.

In some aspects, a method of characterizing a phosphoserine residue of a polypeptide is provided. In some embodiments, the method comprises converting the phosphoserine residue of the polypeptide to a dehydroalanine residue to provide a dehydroalanine-containing polypeptide. In some embodiments, the method comprises reacting the dehydroalanine-containing polypeptide with a thiol compound comprising a click chemistry handle to provide a functionalized polypeptide comprising a functionalized dehydroalanine residue. In some embodiments, the method comprises digesting the functionalized polypeptide to form two or more peptide fragments. In certain embodiments, at least one peptide fragment is a functionalized peptide fragment comprising a functionalized dehydroalanine residue. In some embodiments, the method comprises contacting the functionalized peptide fragment with one or more amino acid recognition molecules. In some embodiments, the method comprises detecting a first series of signal pulses indicative of binding events between the one or more amino acid recognition molecules and a first amino acid at a terminus of the functionalized peptide fragment. In some embodiments, the method comprises determining at least one chemical characteristic of an amino acid of the functionalized peptide fragment based on at least one characteristic of the first series of signal pulses. In some embodiments, the method comprises removing the first amino acid from the terminus of the functionalized peptide fragment. In some embodiments, the method comprises repeating the contacting, detecting, determining, and removing steps until the functionalized dehydroalanine residue is exposed at the terminus of the functionalized peptide fragment.

In some aspects, a method of characterizing a phosphoserine residue of a polypeptide is provided. In some embodiments, the method comprises digesting the polypeptide comprising the phosphoserine residue to form two or more peptide fragments, wherein at least one peptide fragment comprises the phosphoserine residue. In some embodiments, the method comprises converting the phosphoserine residue of the at least one peptide fragment to a dehydroalanine residue of the at least one peptide fragment to provide a dehydroalanine-containing peptide fragment. In some embodiments, the method comprises reacting the dehydroalanine-containing peptide fragment with a thiol compound comprising a click chemistry handle to provide a functionalized peptide fragment comprising a functionalized dehydroalanine residue. In some embodiments, the method comprises contacting the functionalized peptide fragment with one or more amino acid recognition molecules. In some embodiments, the method comprises detecting a first series of signal pulses indicative of binding events between the one or more amino acid recognition molecules and a first amino acid at a terminus of the functionalized peptide fragment. In some embodiments, the method comprises determining at least one chemical characteristic of an amino acid of the functionalized peptide fragment based on at least one characteristic of the first series of signal pulses. In some embodiments, the method comprises removing the first amino acid from the terminus of the functionalized peptide fragment. In some embodiments, the method comprises repeating the contacting, detecting, determining, and removing steps until the functionalized dehydroalanine residue is exposed at the terminus of the functionalized peptide fragment.

The details of certain embodiments of the disclosure are set forth in the Detailed Description. Other features, objects, and advantages of the disclosure will be apparent from the Examples, Drawings, and Claims.

### BRIEF DESCRIPTION OF THE DRAWINGS

The accompanying drawings, which constitute a part of this specification, illustrate several embodiments of the disclosure and, together with the accompanying description, serve to explain the principles of the disclosure.

FIG. 1 shows, according to some embodiments, an example overview of real-time dynamic protein sequencing. Protein samples are digested into peptide fragments, immobilized in nanoscale reaction chambers, and incubated with a mixture of freely-diffusing N-terminal amino acid (NAA) recognizers and aminopeptidases that carry out the sequencing process. The labeled recognizers bind on and off to the peptide when one of their cognate NAAs is exposed at the N-terminus, thereby producing characteristic pulsing patterns. The NAA is cleaved by an aminopeptidase, exposing the next amino acid for recognition. The temporal order of NAA recognition and the kinetics of binding enable peptide characterization and are sensitive to features that modulate binding kinetics, such as post-translational modifications (PTMs).

FIG. 2 shows, according to some embodiments, a schematic illustration of an exemplary system comprising a sample well, where a peptide fragment is immobilized to a bottom surface of the sample well through a target amino acid that is not a terminal amino acid (e.g., a C-terminal amino acid, an N-terminal amino acid) of the peptide fragment.

FIG. 3 shows, according to some embodiments, a schematic illustration of an exemplary pixel of an integrated device.

FIG. 4 shows, according to some embodiments, a schematic illustration of a two-step reaction in which phosphoserine (pSer)-containing proteins are selectively labeled with an azide click chemistry handle and are digested to yield peptide fragments that can be characterized (e.g., sequenced).

FIG. 5A shows, according to some embodiments, a representative single-molecule sequencing trace for pS65-ubiquitin protein. FIG. 5B shows LC-MS/MS data obtained from an Agilent 6550 Quadropole Time-of-Flight (QTOF) showing phosphorylation of Ser 65 of ubiquitin.

FIG. 6A shows, according to some embodiments, an exemplary reaction scheme for selective functionalization of a phosphoserine residue. FIGS. 6B and 6C show, according to some embodiments, representative traces of a phosphoserine-containing fragment of p53 protein. FIG. 6D shows, according to some embodiments, a schematic diagram of selective enrichment of a phosphoserine-containing peptide from a peptide mixture. FIG. 6E shows, according to some embodiments, a representative trace of a sample comprising a 1:1 mix of a phosphoserine-containing fragment of p53 and a non-phosphorylated peptide. Also shown is SEQ ID NO: 24. FIG. 6F shows, according to some embodiments, a representative trace of a mixture of a phosphoserine-containing fragment of p53 and 10 non-phosphorylated peptides. FIG. 6G shows LC-MS/MS data showing phosphorylation of p53.

FIG. 7A shows, according to some embodiments, representative traces for a first phosphoserine-containing fragment of TIF1β. FIG. 7B shows, according to some embodiments, representative traces for a second phosphoserine-containing fragment of TIF1β.

FIG. 8A shows, according to some embodiments, the amino acid sequence of cardiac phospholamban (PLB) and the conversion of phosphorylated cardiac phospholamban (pPLB) to thiol-azide adduct (e.g., functionalization of the phosphoserine residue of pPLB with an azide group). The PLB sequence (SEQ ID NO: 25) is listed as well. FIG. 8B shows, according to some embodiments, a representative LC-MS trace after a 3 hour reaction at room temperature. FIG. 8C shows, according to some embodiments, LC-MS data showing conversion of pPLB to thiol-azide adduct.

FIG. 9A shows, according to some embodiments, sequencing of pPLB anchored with Thiol-PEG-azide (e.g., digestion, functionalization of the phosphoserine residue with an azide group, and conjugation to linker-SV), shown as SEQ ID NO: 26. FIG. 9B shows, according to some embodiments, representative traces for a phosphoserine-containing fragment of pPLB.

### DEFINITIONS

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Michael B. Smith, March's Advanced Organic Chemistry, 7^{th} Edition, John Wiley & Sons, Inc., New York, 2013; Richard C. Larock, Comprehensive Organic Transformations, John Wiley & Sons, Inc., New York, 2018; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, N Y, 1962); and Wilen, S. H., Tables of Resolving Agents and Optical Resolutions p. 268 (E. L. Eliel, Ed., Univ. of Notre Dame Press, Notre Dame, IN 1972). The invention additionally encompasses compounds as individual isomers substantially free of other isomers, and alternatively, as mixtures of various isomers.

Unless otherwise provided, formulae and structures depicted herein include compounds that do not include isotopically enriched atoms, and also include compounds that include isotopically enriched atoms. For example, compounds having the present structures except for the replacement of hydrogen by deuterium or tritium, replacement of ¹⁹F with ¹⁸F, or the replacement of a carbon by a ¹³C- or ¹⁴C-enriched carbon are within the scope of the disclosure. Such compounds are useful, for example, as analytical tools or probes in biological assays.

When a range of values (“range”) is listed, it encompasses each value and sub-range within the range. A range is inclusive of the values at the two ends of the range unless otherwise provided. For example, “C₁₋₆ alkyl” encompasses C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

When a range of values (“range”) is listed, it encompasses each value and sub-range within the range. A range is inclusive of the values at the two ends of the range unless otherwise provided. For example “C₁₋₆ alkyl” encompasses, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”), 1 to 10 carbon atoms (“C₁₋₁₀ alkyl”), 1 to 9 carbon atoms (“C₁₋₉ alkyl”), 1 to 8 carbon atoms (“C₁₋₈ alkyl”), 1 to 7 carbon atoms (“C₁₋₇ alkyl”), 1 to 6 carbon atoms (“C₁₋₆ alkyl”), 1 to 5 carbon atoms (“C₁₋₅ alkyl”), 1 to 4 carbon atoms (“C₁₋₄ alkyl”), 1 to 3 carbon atoms (“C₁₋₃ alkyl”), 1 to 2 carbon atoms (“C₁₋₂ alkyl”), or 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tert-amyl), and hexyl (C₆) (e.g., n-hexyl). Additional examples of alkyl groups include n-heptyl (C₇), n-octyl (C₈), n-dodecyl (C₁₂), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₂ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is a substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)).

The term “haloalkyl” is a substituted alkyl group, wherein one or more of the hydrogen atoms are independently replaced by a halogen, e.g., fluoro, bromo, chloro, or iodo. “Perhaloalkyl” is a subset of haloalkyl, and refers to an alkyl group wherein all of the hydrogen atoms are independently replaced by a halogen, e.g., fluoro, bromo, chloro, or iodo. In some embodiments, the haloalkyl moiety has 1 to 20 carbon atoms (“C₁₋₂₀ haloalkyl”), 1 to 10 carbon atoms (“C₁₋₁₀ haloalkyl”), 1 to 9 carbon atoms (“C₁₋₉ haloalkyl”), 1 to 8 carbon atoms (“C₁₋₈ haloalkyl”), 1 to 7 carbon atoms (“C₁₋₇ haloalkyl”), 1 to 6 carbon atoms (“C₁₋₆ haloalkyl”), 1 to 5 carbon atoms (“C₁₋₅ haloalkyl”), 1 to 4 carbon atoms (“C₁₋₄ haloalkyl”), 1 to 3 carbon atoms (“C₁₋₃ haloalkyl”), or 1 to 2 carbon atoms (“C₁₋₂ haloalkyl”). In some embodiments, all of the haloalkyl hydrogen atoms are independently replaced with fluoro to provide a “perfluoroalkyl” group. In some embodiments, all of the haloalkyl hydrogen atoms are independently replaced with chloro to provide a “perchloroalkyl” group. Examples of haloalkyl groups include —CHF₂, —CH₂F, —CF₃, —CH₂CF₃, —CF₂CF₃, —CF₂CF₂CF₃, —CCl₃, —CFCl₂, —CF₂Cl, and the like.

The term “heteroalkyl” refers to an alkyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl”). In certain embodiments, a heteroalkyl group refers to a saturated group having from 1 to 12 carbon atoms and 1 or more heteroatoms within the parent chain (“heteroC₁₋₁₂ alkyl”). In some embodiments, a heteroalkyl group is a saturated group having 1 to 11 carbon atoms and 1 or more heteroatoms within the parent chain (“heteroC₁₋₁₁ alkyl”). In some embodiments, a heteroalkyl group is a saturated group having 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁-s alkyl”). In some embodiments, a heteroalkyl group is a saturated group having 1 to 4 carbon atoms and for 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₂ alkyl. In certain embodiments, the heteroalkyl group is a substituted heteroC₁₋₁₂ alkyl.

The term “alkenyl” refers to a radical of a straight-chain or branched hydrocarbon group having from 1 to 20 carbon atoms and one or more carbon-carbon double bonds (e.g., 1, 2, 3, or 4 double bonds). In some embodiments, an alkenyl group has 1 to 20 carbon atoms (“C₁₋₂₀ alkenyl”). In some embodiments, an alkenyl group has 1 to 12 carbon atoms (“C₁₋₁₂ alkenyl”). In some embodiments, an alkenyl group has 1 to 11 carbon atoms (“C₁₋₁₁ alkenyl”). In some embodiments, an alkenyl group has 1 to 10 carbon atoms (“C₁₋₁₀ alkenyl”). In some embodiments, an alkenyl group has 1 to 9 carbon atoms (“C₁₋₉ alkenyl”). In some embodiments, an alkenyl group has 1 to 8 carbon atoms (“C₁₋₈ alkenyl”). In some embodiments, an alkenyl group has 1 to 7 carbon atoms (“C₁₋₇ alkenyl”). In some embodiments, an alkenyl group has 1 to 6 carbon atoms (“C₁₋₆ alkenyl”). In some embodiments, an alkenyl group has 1 to 5 carbon atoms (“C₁₋₅ alkenyl”). In some embodiments, an alkenyl group has 1 to 4 carbon atoms (“C₁₋₄ alkenyl”). In some embodiments, an alkenyl group has 1 to 3 carbon atoms (“C₁₋₃ alkenyl”). In some embodiments, an alkenyl group has 1 to 2 carbon atoms (“C₁₋₂ alkenyl”). In some embodiments, an alkenyl group has 1 carbon atom (“C₁ alkenyl”). The one or more carbon-carbon double bonds can be internal (such as in 2-butenyl) or terminal (such as in 1-butenyl). Examples of C₁₋₄ alkenyl groups include methylidenyl (C₁), ethenyl (C₂), 1-propenyl (C₃), 2-propenyl (C₃), 1-butenyl (C₄), 2-butenyl (C₄), butadienyl (C₄), and the like. Examples of C₁₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₁₋₂₀ alkenyl. In certain embodiments, the alkenyl group is a substituted C₁₋₂₀ alkenyl. In an alkenyl group, a C═C double bond for which the stereochemistry is not specified (e.g., —CH═CHCH₃ or

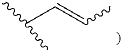

may be in the (E)- or (Z)-configuration.

The term “heteroalkenyl” refers to an alkenyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkenyl group refers to a group having from 1 to 20 carbon atoms, at least one double bond, and 1 or more heteroatoms within the parent chain (“heteroC₁₋₂₀ alkenyl”). In certain embodiments, a heteroalkenyl group refers to a group having from 1 to 12 carbon atoms, at least one double bond, and 1 or more heteroatoms within the parent chain (“heteroC₁₋₁₂ alkenyl”). In certain embodiments, a heteroalkenyl group refers to a group having from 1 to 11 carbon atoms, at least one double bond, and 1 or more heteroatoms within the parent chain (“heteroC₁₋₁₁ alkenyl”). In certain embodiments, a heteroalkenyl group refers to a group having from 1 to 10 carbon atoms, at least one double bond, and 1 or more heteroatoms within the parent chain (“heteroC₁₋₁₀ alkenyl”). In some embodiments, a heteroalkenyl group has 1 to 9 carbon atoms at least one double bond, and 1 or more heteroatoms within the parent chain (“heteroC₁₋₉ alkenyl”). In some embodiments, a heteroalkenyl group has 1 to 8 carbon atoms, at least one double bond, and 1 or more heteroatoms within the parent chain (“heteroC₁₋₈ alkenyl”). In some embodiments, a heteroalkenyl group has 1 to 7 carbon atoms, at least one double bond, and 1 or more heteroatoms within the parent chain (“heteroC₁₋₇ alkenyl”). In some embodiments, a heteroalkenyl group has 1 to 6 carbon atoms, at least one double bond, and 1 or more heteroatoms within the parent chain (“heteroC₁₋₆ alkenyl”). In some embodiments, a heteroalkenyl group has 1 to carbon atoms, at least one double bond, and 1 or 2 heteroatoms within the parent chain (“heteroC₁-s alkenyl”). In some embodiments, a heteroalkenyl group has 1 to 4 carbon atoms, at least one double bond, and 1 or 2 heteroatoms within the parent chain (“heteroC₁₋₄ alkenyl”). In some embodiments, a heteroalkenyl group has 1 to 3 carbon atoms, at least one double bond, and 1 heteroatom within the parent chain (“heteroC₁₋₃ alkenyl”). In some embodiments, a heteroalkenyl group has 1 to 2 carbon atoms, at least one double bond, and 1 heteroatom within the parent chain (“heteroC₁₋₂ alkenyl”). In some embodiments, a heteroalkenyl group has 1 to 6 carbon atoms, at least one double bond, and 1 or 2 heteroatoms within the parent chain (“heteroC₁₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₁₋₂₀ alkenyl. In certain embodiments, the heteroalkenyl group is a substituted heteroC₁₋₂₀ alkenyl.

The term “alkynyl” refers to a radical of a straight-chain or branched hydrocarbon group having from 1 to 20 carbon atoms and one or more carbon-carbon triple bonds (e.g., 1, 2, 3, or 4 triple bonds) (“C₁₋₂₀ alkynyl”). In some embodiments, an alkynyl group has 1 to 10 carbon atoms (“C₁₋₁₀ alkynyl”). In some embodiments, an alkynyl group has 1 to 9 carbon atoms (“C₁₋₉ alkynyl”). In some embodiments, an alkynyl group has 1 to 8 carbon atoms (“C₁₋₈ alkynyl”). In some embodiments, an alkynyl group has 1 to 7 carbon atoms (“C₁₋₇ alkynyl”). In some embodiments, an alkynyl group has 1 to 6 carbon atoms (“C₁₋₆ alkynyl”). In some embodiments, an alkynyl group has 1 to 5 carbon atoms (“Cis alkynyl”). In some embodiments, an alkynyl group has 1 to 4 carbon atoms (“C₁₋₄ alkynyl”). In some embodiments, an alkynyl group has 1 to 3 carbon atoms (“C₁₋₃ alkynyl”). In some embodiments, an alkynyl group has 1 to 2 carbon atoms (“C₁₋₂ alkynyl”). In some embodiments, an alkynyl group has 1 carbon atom (“C₁ alkynyl”). The one or more carbon-carbon triple bonds can be internal (such as in 2-butynyl) or terminal (such as in 1-butynyl). Examples of C₁₋₄ alkynyl groups include, without limitation, methylidynyl (C₁), ethynyl (C₂), 1-propynyl (C₃), 2-propynyl (C₃), 1-butynyl (C₄), 2-butynyl (C₄), and the like. Examples of C₁₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₁₋₂₀ alkynyl. In certain embodiments, the alkynyl group is a substituted C₁₋₂₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkynyl group refers to a group having from 1 to 20 carbon atoms, at least one triple bond, and 1 or more heteroatoms within the parent chain (“heteroC₁₋₂₀ alkynyl”). In certain embodiments, a heteroalkynyl group refers to a group having from 1 to 10 carbon atoms, at least one triple bond, and 1 or more heteroatoms within the parent chain (“heteroC₁₋₁₀ alkynyl”). In some embodiments, a heteroalkynyl group has 1 to 9 carbon atoms, at least one triple bond, and 1 or more heteroatoms within the parent chain (“heteroC₁₋₉ alkynyl”). In some embodiments, a heteroalkynyl group has 1 to 8 carbon atoms, at least one triple bond, and 1 or more heteroatoms within the parent chain (“heteroC₁₋₈ alkynyl”). In some embodiments, a heteroalkynyl group has 1 to 7 carbon atoms, at least one triple bond, and 1 or more heteroatoms within the parent chain (“heteroC₁₋₇ alkynyl”). In some embodiments, a heteroalkynyl group has 1 to 6 carbon atoms, at least one triple bond, and 1 or more heteroatoms within the parent chain (“heteroC₁₋₆ alkynyl”). In some embodiments, a heteroalkynyl group has 1 to 5 carbon atoms, at least one triple bond, and 1 or 2 heteroatoms within the parent chain (“heteroC₁-s alkynyl”). In some embodiments, a heteroalkynyl group has 1 to 4 carbon atoms, at least one triple bond, and for 2 heteroatoms within the parent chain (“heteroC₁₋₄ alkynyl”). In some embodiments, a heteroalkynyl group has 1 to 3 carbon atoms, at least one triple bond, and 1 heteroatom within the parent chain (“heteroC₁₋₃ alkynyl”). In some embodiments, a heteroalkynyl group has 1 to 2 carbon atoms, at least one triple bond, and 1 heteroatom within the parent chain (“heteroC₁₋₂ alkynyl”). In some embodiments, a heteroalkynyl group has 1 to 6 carbon atoms, at least one triple bond, and 1 or 2 heteroatoms within the parent chain (“heteroC₁₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₁₋₂₀ alkynyl. In certain embodiments, the heteroalkynyl group is a substituted heteroC₁₋₂₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 14 ring carbon atoms (“C₃₋₁₄ carbocyclyl”). In some embodiments, a carbocyclyl group has 3 to 13 ring carbon atoms (“C₃₋₁₃ carbocyclyl”). In some embodiments, a carbocyclyl group has 3 to 12 ring carbon atoms (“C₃₋₁₂ carbocyclyl”). In some embodiments, a carbocyclyl group has 3 to 11 ring carbon atoms (“C₃₋₁₁ carbocyclyl”). In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. Exemplary C₃₋₈ carbocyclyl groups include the aforementioned C₃₋₁₀ carbocyclyl groups as well as cycloundecyl (C₁₁), spiro[5.5]undecanyl (C₁₁), cyclododecyl (C₁₂), cyclododecenyl (C₁₂), cyclotridecane (C₁₃), cyclotetradecane (C₁₄), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl. In certain embodiments, the carbocyclyl includes 0, 1, or 2 C═C double bonds in the carbocyclic ring system, as valency permits.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl. In certain embodiments, the heterocyclyl is substituted or unsubstituted, 3- to 7-membered, monocyclic heterocyclyl, wherein 1, 2, or 3 atoms in the heterocyclic ring system are independently oxygen, nitrogen, or sulfur, as valency permits.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1 4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include azirdinyl, oxiranyl, and thiiranyl. Exemplary 4-membered heterocyclyl groups containing 1 heteroatom include azetidinyl, oxetanyl, and thietanyl. Exemplary 5-membered heterocyclyl groups containing 1 heteroatom include tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include dioxolanyl, oxathiolanyl and dithiolanyl. Exemplary 5-membered heterocyclyl groups containing 3 heteroatoms include triazolinyl, oxadiazolinyl, and thiadiazolinyl. Exemplary 6-membered heterocyclyl groups containing 1 heteroatom include piperidinyl, tetrahydropyranyl, dihydropyridinyl, and thianyl. Exemplary 6-membered heterocyclyl groups containing 2 heteroatoms include piperazinyl, morpholinyl, dithianyl, and dioxanyl. Exemplary 6-membered heterocyclyl groups containing 3 heteroatoms include triazinyl. Exemplary 7-membered heterocyclyl groups containing 1 heteroatom include azepanyl, oxepanyl and thiepanyl. Exemplary 8-membered heterocyclyl groups containing 1 heteroatom include azocanyl, oxecanyl and thiocanyl. Exemplary bicyclic heterocyclyl groups include indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 (electrons shared in a cyclic array) having 6 14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Aralkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14 (electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, e.g., either the ring bearing a heteroatom (e.g., 2-indolyl) or the ring that does not contain a heteroatom (e.g., 5-indolyl). In certain embodiments, the heteroaryl is substituted or unsubstituted, 5- or 6-membered, monocyclic heteroaryl, wherein 1, 2, 3, or 4 atoms in the heteroaryl ring system are independently oxygen, nitrogen, or sulfur. In certain embodiments, the heteroaryl is substituted or unsubstituted, 9- or 10-membered, bicyclic heteroaryl, wherein 1, 2, 3, or 4 atoms in the heteroaryl ring system are independently oxygen, nitrogen, or sulfur.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include pyrrolyl, furanyl, and thiophenyl. Exemplary 5-membered heteroaryl groups containing 2 heteroatoms include imidazolyl, pyrazolyl, oxazolyl, isoxazolyl, thiazolyl, and isothiazolyl. Exemplary 5-membered heteroaryl groups containing 3 heteroatoms include triazolyl, oxadiazolyl, and thiadiazolyl. Exemplary 5-membered heteroaryl groups containing 4 heteroatoms include tetrazolyl. Exemplary 6-membered heteroaryl groups containing 1 heteroatom include pyridinyl. Exemplary 6-membered heteroaryl groups containing 2 heteroatoms include pyridazinyl, pyrimidinyl, and pyrazinyl. Exemplary 6-membered heteroaryl groups containing 3 or 4 heteroatoms include triazinyl and tetrazinyl, respectively. Exemplary 7-membered heteroaryl groups containing 1 heteroatom include azepinyl, oxepinyl, and thiepinyl. Exemplary 5,6-bicyclic heteroaryl groups include indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include naphthyridinyl, pteridinyl, quinolinyl, isoquinolinyl, cinnolinyl, quinoxalinyl, phthalazinyl, and quinazolinyl. Exemplary tricyclic heteroaryl groups include phenanthridinyl, dibenzofuranyl, carbazolyl, acridinyl, phenothiazinyl, phenoxazinyl, and phenazinyl.

“Heteroaralkyl” is a subset of “alkyl” and refers to an alkyl group substituted by a heteroaryl group, wherein the point of attachment is on the alkyl moiety.

The term “unsaturated bond” refers to a double or triple bond.

The term “unsaturated” or “partially unsaturated” refers to a moiety that includes at least one double or triple bond.

The term “saturated” or “fully saturated” refers to a moiety that does not contain a double or triple bond, e.g., the moiety only contains single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which is substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety. The invention is not limited in any manner by the exemplary substituents described herein.

Exemplary carbon atom substituents include halogen, —CN, —NO₂, —N₃, —SO₂H, —SO₃H, —OH, —OR^{aa}, —ON(R^{bb})₂, —N(R^{bb})₂, —N(R)₃⁺X⁻, —N(OR^{cc})R^{bb}, —SH, —SR^{aa}, —SSR^{cc}, —C(═O)R^{aa}, —CO₂H, —CHO, —C(OR)₂, —CO₂R^{aa}, —OC(═O)R^{aa}, —OCO₂R′, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂—NR^{bb}P(═O)(R^{aa})₂—, NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃X, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};
wherein:
   each instance of R^{aa} is, independently, selected from C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each of the alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{bb} is, independently, selected from hydrogen, —OH, —OR^{aa}, —N(R^{cc})₂, —CN, —C(═O)R^{aa}, —C(═O)N(R)₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R)₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, heteroC₁₋₂₀alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{cc} is, independently, selected from hydrogen, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{dd} is, independently, selected from halogen, —CN, —NO₂, —N₃, SO₂H, —SO₃H, —OH, —OR^{ee}, —ON(R^{ff})₂, —N(R^{ff})₂, —N(R^{ff})₃X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{a})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{f}SO₂R^{ee}, —SO₂N(R^{ff})₂, —SO₂R^{ee}, —SO₂OR^{ee}, —OSO₂R^{ee}, —S(═O)R^{ee}, —Si(R)₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₁₀ alkyl, C₁₋₁₀ perhaloalkyl, C₁₋₁₀ alkenyl, C₁₋₁₀ alkynyl, heteroC₁₋₁₀alkyl, heteroC₁₋₁₀alkenyl, heteroC₁₋₁₀alkynyl, C₃₋₁₀ carbocyclyl, 3-10 membered heterocyclyl, C₆₋₁₀ aryl, and 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R⁹⁹ groups, or two geminal R^{dd} substituents are joined to form ═O or ═S; wherein X⁻ is a counterion;
   each instance of R is, independently, selected from C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R⁹⁹ groups;
   each instance of R^{ff} is, independently, selected from hydrogen, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, 3-10 membered heterocyclyl, C₆₋₁₀ aryl, and 5-10 membered heteroaryl, or two R^{a} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R⁹⁹ groups;
   each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃—C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, or 5-10 membered heteroaryl; or two geminal R⁹⁹ substituents can be joined to form ═O or ═S; and each X⁻ is a counterion.

In certain embodiments, each carbon atom substituent is independently halogen, substituted (e.g., substituted with one or more halogen) or unsubstituted C₁₋₆ alkyl, —OR^{aa}—SR^{aa}—N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R⁻, or —NR^{bb}C(═O)N(R^{bb})₂. In certain embodiments, each carbon atom substituent is independently halogen, substituted (e.g., substituted with one or more halogen) or unsubstituted C₁₋₁₀ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts). In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, or —NO₂. In certain embodiments, each carbon atom substituent is independently halogen, substituted (e.g., substituted with one or more halogen moieties) or unsubstituted C₁₋₁₀ alkyl, —OR^{aa}, —SR^{aa}, —N(R^{bb})₂, —CN, —SCN, or —NO₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts).

In certain embodiments, the molecular weight of a carbon atom substituent is lower than 250, lower than 200, lower than 150, lower than 100, or lower than 50 g/mol. In certain embodiments, a carbon atom substituent consists of carbon, hydrogen, fluorine, chlorine, bromine, iodine, oxygen, sulfur, nitrogen, and/or silicon atoms. In certain embodiments, a carbon atom substituent consists of carbon, hydrogen, fluorine, chlorine, bromine, iodine, oxygen, sulfur, and/or nitrogen atoms. In certain embodiments, a carbon atom substituent consists of carbon, hydrogen, fluorine, chlorine, bromine, and/or iodine atoms. In certain embodiments, a carbon atom substituent consists of carbon, hydrogen, fluorine, and/or chlorine atoms.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R)₂, —OP(R^{cc})₃⁺X⁻, —OP(OR^{cc})₂, —OP(OR^{cc})₃⁺X⁻, —OP(═O)(R^{aa})₂, —OP(═O)(OR^{cc})₂, and —OP(═O)(N(R^{bb}))₂, wherein X⁻, R^{aa}R^{bb} and R^{cc} are as defined herein.

The term “thiol” or “thio” refers to the group —SH. The term “substituted thiol” or “substituted thio,” by extension, refers to a thiol group wherein the sulfur atom directly attached to the parent molecule is substituted with a group other than hydrogen, and includes groups selected from —SR, —S═SR^{cc}, —SC(═S)SR^{aa}, —SC(═S)OR^{aa}, —SC(═S) N(R^{bb})₂, —SC(═O)SR^{aa}, —SC(═O)OR^{aa}, —SC(═O)N(R^{bb})₂, and —SC(═O)R^{aa}, wherein R^{aa} and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group.

The term “mono substituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(N(R^{bb})₂)₂, wherein R^{aa}R^{bb} and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb} C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR)₂, and —NR^{bb}P(═O)(N(R^{bb})₂)₂, wherein R^{aa}, R^{bb}, and R^{cc} are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and X⁻ are as defined herein.

The term “sulfonyl” refers to a group selected from —SO₂N(R^{bb})₂, —SO₂R^{aa}, and —SO₂OR^{aa} wherein R^{aa} and R^{bb} are as defined herein.

The term “sulfinyl” refers to the group —S(═O)R^{aa}, wherein R^{aa} is as defined herein.

The term “acyl” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, and —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “carbonyl” refers to a group wherein the carbon directly attached to the parent molecule is sp² hybridized, and is substituted with an oxygen, nitrogen or sulfur atom, e.g., a group selected from ketones (—C(═O)R^{aa}), carboxylic acids (—CO₂H), aldehydes (—CHO), esters (—CO₂R^{aa}, —C(═O)SR^{aa}, —C(═S)SR^{aa}), amides (—C(═O)N(R^{bb})₂, —C(═O)NR^{bb}SO₂R^{aa}, —C(═S)N(R^{bb})₂), and imines (—C(═NR^{bb})R^{aa}, —C(═NR^{bb})OR^{aa}), —C(═NR^{bb})N(R^{bb})₂), wherein R and R^{bb} are as defined herein.

The term “silyl” refers to the group —Si(R^{aa})₃, wherein R^{aa} is as defined herein.

The term “phosphino” refers to the group —P(R^{cc})₂, wherein R^{cc} is as defined herein.

The term “oxo” refers to the group ═O, and the term “thiooxo” refers to the group ═S.

In certain embodiments, the molecular weight of a substituent is lower than 250, lower than 200, lower than 150, lower than 100, or lower than 50 g/mol. In certain embodiments, a substituent consists of carbon, hydrogen, fluorine, chlorine, bromine, iodine, oxygen, sulfur, nitrogen, and/or silicon atoms. In certain embodiments, a substituent consists of carbon, hydrogen, fluorine, chlorine, bromine, iodine, oxygen, sulfur, and/or nitrogen atoms. In certain embodiments, a substituent consists of carbon, hydrogen, fluorine, chlorine, bromine, and/or iodine atoms. In certain embodiments, a substituent consists of carbon, hydrogen, fluorine, and/or chlorine atoms. In certain embodiments, a substituent comprises 0, 1, 2, or 3 hydrogen bond donors. In certain embodiments, a substituent comprises 0, 1, 2, or 3 hydrogen bond acceptors.

A “counterion” or “anionic counterion” is a negatively charged group associated with a positively charged group in order to maintain electronic neutrality. An anionic counterion may be monovalent (e.g., including one formal negative charge). An anionic counterion may also be multivalent (e.g., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂⁻ or (HCB₁₁MesBr₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

Use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

A “non-hydrogen group” refers to any group that is defined for a particular variable that is not hydrogen.

These and other exemplary substituents are described in more detail in the Detailed Description, Examples, and Claims. The invention is not limited in any manner by the above exemplary listing of substituents.

As used herein, the term “salt” refers to any and all salts and encompasses pharmaceutically acceptable salts. Salts include ionic compounds that result from the neutralization reaction of an acid and a base. A salt is composed of one or more cations (positively charged ions) and one or more anions (negative ions) so that the salt is electrically neutral (without a net charge). Salts of the compounds of this invention include those derived from inorganic and organic acids and bases. Examples of acid addition salts are salts of an amino group formed with inorganic acids, such as hydrochloric acid, hydrobromic acid, phosphoric acid, sulfuric acid, and perchloric acid, or with organic acids, such as acetic acid, oxalic acid, maleic acid, tartaric acid, citric acid, succinic acid, or malonic acid or by using other methods known in the art such as ion exchange. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate, hippurate, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further salts include ammonium, quaternary ammonium, and amine cations formed using counterions such as halide, hydroxide, carboxylate, sulfate, phosphate, nitrate, lower alkyl sulfonate, and aryl sulfonate.

As used herein, the term “work up” refers to any single step or series of multiple steps relating to isolating and/or purifying one or more products of a chemical reaction (e.g., from any remaining starting material, other reagents, solvents, or byproducts of the chemical reaction). Working up a reaction may include removing solvents by, for example, evaporation or lyophilization. Working up a reaction may also include performing liquid-liquid extraction, for example, by separating the reaction mixture into organic and aqueous layers. In some embodiments, working up a reaction includes quenching the reaction to deactivate any unreacted reagents. Working up a reaction may also include cooling a reaction mixture to induce precipitation of solids from the mixture, which may be collected or removed by, for example, filtration, decantation, or centrifugation. Working up a reaction can also include purifying one or more products of the reaction by chromatography. Other methods may also be used to purify one or more reaction products, including, but not limited to, distillation and recrystallization. Other processes for working up a reaction are known in the art, and a person of ordinary skill in the art would readily be capable of determining other appropriate methods that could be employed in working up a particular reaction.

As used herein, the term “about X,” or “approximately X,” where X is a number or percentage, refers to a number or percentage that is between 99.5% and 100.5%, between 99% and 101%, between 98% and 102%, between 97% and 103%, between 96% and 104%, between 95% and 105%, between 92% and 108%, or between 90% and 110%, inclusive, of X.

The terms “polynucleotide”, “nucleotide sequence”, “nucleic acid”, “nucleic acid molecule”, “nucleic acid sequence”, and “oligonucleotide” refer to a series of nucleotide bases (also called “nucleotides”) in DNA and RNA, and mean any chain of two or more nucleotides. The polynucleotides can be chimeric mixtures or derivatives or modified versions thereof, single-stranded or double-stranded. The oligonucleotide can be modified at the base moiety, sugar moiety, or phosphate backbone, for example, to improve stability of the molecule, its hybridization parameters, etc. The antisense oligonuculeotide may comprise a modified base moiety which is selected from the group including, but not limited to, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N₆-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N₆-isopentenyladenine, wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid, 5-methyl-2-thiouracil, 3-(3-amino-3-N-2-carboxypropyl) uracil, a thio-guanine, and 2,6-diaminopurine. A nucleotide sequence typically carries genetic information, including the information used by cellular machinery to make proteins and enzymes. These terms include double- or single-stranded genomic and cDNA, RNA, any synthetic and genetically manipulated polynucleotide, and both sense and antisense polynucleotides. This includes single- and double-stranded molecules, i.e., DNA-DNA, DNA-RNA and RNA-RNA hybrids, as well as “protein nucleic acids” (PNAs) formed by conjugating bases to an amino acid backbone. This also includes nucleic acids containing carbohydrate or lipids. Exemplary DNAs include single-stranded DNA (ssDNA), double-stranded DNA (dsDNA), plasmid DNA (pDNA), genomic DNA (gDNA), complementary DNA (cDNA), antisense DNA, chloroplast DNA (ctDNA or cpDNA), microsatellite DNA, mitochondrial DNA (mtDNA or mDNA), kinetoplast DNA (kDNA), provirus, lysogen, repetitive DNA, satellite DNA, and viral DNA. Exemplary RNAs include single-stranded RNA (ssRNA), double-stranded RNA (dsRNA), small interfering RNA (siRNA), messenger RNA (mRNA), precursor messenger RNA (pre-mRNA), small hairpin RNA or short hairpin RNA (shRNA), microRNA (miRNA), guide RNA (gRNA), transfer RNA (tRNA), antisense RNA (asRNA), heterogeneous nuclear RNA (hnRNA), coding RNA, non-coding RNA (ncRNA), long non-coding RNA (long ncRNA or lncRNA), satellite RNA, viral satellite RNA, signal recognition particle RNA, small cytoplasmic RNA, small nuclear RNA (snRNA), ribosomal RNA (rRNA), Piwi-interacting RNA (piRNA), polyinosinic acid, ribozyme, flexizyme, small nucleolar RNA (snoRNA), spliced leader RNA, viral RNA, and viral satellite RNA.

A “protein,” “peptide,” or “polypeptide” comprises a polymer of amino acid residues linked together by peptide bonds. The term refers to proteins, polypeptides, and peptides of any size, structure, or function. Typically, a protein will be at least three amino acids long. A protein may refer to an individual protein or a collection of proteins. Inventive proteins preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in a protein may be modified, for example, by the addition of a chemical entity such as a carbohydrate group, a hydroxyl group, a phosphate group, a farnesyl group, an isofarnesyl group, a fatty acid group, a linker for conjugation or functionalization, or other modification. A protein may also be a single molecule or may be a multi-molecular complex. A protein may be a fragment of a naturally occurring protein or peptide. A protein may be naturally occurring, recombinant, synthetic, or any combination of these.

Amino acid residues may be indicated by their corresponding single letter codes, e.g., R (arginine), H (histidine), K (lysine), D (aspartic acid), E (glutamic acid), S (serine), T (threonine), N (asparagine), Q (glutamine), C (cysteine), G (glycine), P (proline), A (alanine), V (valine), I (isoleucine), L (leucine), M (methionine), F (phenylalanine), Y (tyrosine), W (tryptophan).

### DETAILED DESCRIPTION

According to some aspects, systems and methods for characterizing phosphoserine-containing polypeptides are described. In some embodiments, a phosphoserine residue of a polypeptide or peptide fragment may be converted to a dehydroalanine residue. Some aspects are directed to a method of reacting a polypeptide comprising at least one dehydroalanine residue with a protected thiol compound comprising a click chemistry handle (e.g., an azide, a tetrazine, a nitrile oxide, an alkyne, or an alkene) to provide a functionalized polypeptide comprising a click chemistry handle. Some aspects are directed to a method of reacting a peptide fragment comprising at least one dehydroalanine residue with a protected thiol compound comprising a click chemistry handle (e.g., an azide, a tetrazine, a nitrile oxide, an alkyne, or an alkene) to provide a functionalized peptide fragment comprising a click chemistry handle. Some aspects are directed to systems and methods of characterizing phosphoserine residues in polypeptides (e.g., identifying and/or determining the location of a phosphoserine residue) and/or methods of preparing samples for such systems and methods.

Certain aspects are directed to a bioconjugation reaction that selectively targets phosphoserine residues and functionalizes them with a click chemistry handle. In some cases, a phosphoserine residue of a polypeptide or a peptide fragment may be converted to a dehydroalanine residue (e.g., through reaction with one or more bases). Surprisingly, the inventors discovered that a dehydroalanine residue can be selectively reacted with a protected thiol compound comprising a click chemistry handle to produce a functionalized polypeptide comprising the click chemistry handle or a functionalized peptide fragment comprising the click chemistry handle. Conventional methods of functionalizing dehydroalanine residues generally require reaction with an unprotected thiol compound (e.g., a compound having a free —SH group) and/or an amine compound. These conventional methods may be associated with certain disadvantages, such as requiring large amounts of the unprotected thiol compound or amine compound. In some cases, for example, these conventional methods may require a molar ratio of the unprotected thiol compound or amine compound to a dehydroalanine-containing polypeptide in a range from 1000:1 to more than 10,000:1. Without wishing to be bound by any particular theory, such large molar ratios may be required due to oxidation of unprotected thiol compounds over time and/or relatively low reactivity of amine compounds with dehydroalanine residues. In contrast, a protected thiol compound generally will not oxidize prior to reaction with a dehydroalanine residue and may exhibit relatively high reactivity. As a result, much lower molar ratios (e.g., about 2:1 or less) of the protected thiol compound to the dehydroalanine-containing polypeptide may be needed to achieve functionalization of the dehydroalanine-containing polypeptide with a click chemistry handle. These low molar ratios may advantageously reduce waste and cost associated with these reactions. In addition, the use of protected thiol compounds, which are often odorless, may avoid other disadvantages associated with the use of unprotected thiol compounds and/or amine compounds in conventional methods, such as strong, unpleasant odor.

Some aspects are directed to a method comprising reacting a polypeptide comprising at least one dehydroalanine residue with a protected thiol compound of Formula (I):

R₁-(L₁)ₘ-S—X (I),
or a salt thereof, wherein:
   X is a protecting group;
   L₁ is a linking group;
   m is an integer from 0 to 36; and
   R₁ is a moiety comprising a click chemistry handle.

In some embodiments, this provides a functionalized polypeptide comprising a functionalized residue of Formula (II):

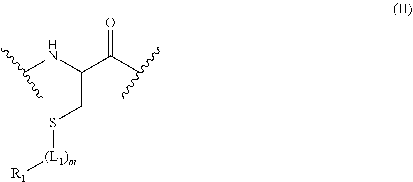

or a salt thereof.

Some aspects are directed to a method comprising reacting a peptide fragment comprising at least one dehydroalanine residue with a protected thiol compound of Formula (I):

R₁-(L₁)ₘ-S—X (I),
or a salt thereof, wherein:
   X is a protecting group;
   L₁ is a linking group;
   m is an integer from 0 to 36; and
   R₁ is a moiety comprising a click chemistry handle.

In some embodiments, this provides a functionalized peptide fragment comprising a functionalized residue of Formula (II):

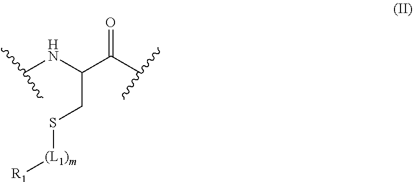

or a salt thereof.

In some embodiments, X is a protecting group. A “protecting group” generally refers to an atomic or molecular fragment that departs with a pair of electrons in heterolytic bond cleavage, wherein the molecular fragment is an anion or neutral molecule. As used herein, a protecting group can be an atom or a group capable of being displaced by a nucleophile.

In some embodiments, X is selected from the group consisting of acetyl, allyl, propargyl, succinimide, thioether, thioester, glycosyl, alkenyl, diphenylmethyl, tetrahydropyranyl, 9-fluorenylmethyl, 9H-xanthen-9-yl, pseudoproline, tert-butyl, urea, aryl, benzyl, trityl, and disulfide.

In some embodiments, L₁ is a linking group. In some embodiments, L₁ is selected from the group consisting of substituted or unsubstituted alkylene, substituted or unsubstituted alkenylene, substituted or unsubstituted alkynylene, substituted or unsubstituted heteroalkylene, substituted or unsubstituted heteroalkenylene, substituted or unsubstituted heteroalkynylene, substituted or unsubstituted carbocyclylene, substituted or unsubstituted heterocyclylene, substituted or unsubstituted arylene, substituted or unsubstituted heteroarylene, or any combination thereof. In certain embodiments, L₁ comprises a substituted or unsubstituted heteroalkylene. In some instances, the substituted or unsubstituted heteroalkylene comprises an alkylene oxide. A non-limiting example of a suitable alkylene oxide is polyethylene glycol.

In some embodiments, m is an integer from 0 to 36. In certain embodiments, m is an integer in a range from 0 to 3, 0 to 5, 0 to 10, 0 to 15, 0 to 20, 0 to 25, 0 to 30, 0 to 36, 1 to 3, 1 to 5, 1 to 10, 1 to 15, 1 to 20, 1 to 25, 1 to 30, 1 to 36, 3 to 5, 3 to 10, 3 to 15, 3 to 20, 3 to 25, 3 to 30, 3 to 36, 5 to 10, 5 to 15, 5 to 20, 5 to 25, 5 to 30, 5 to 36, 10 to 15, 10 to 20, 10 to 25, 10 to 30, 10 to 36, 15 to 20, 15 to 25, 15 to 30, 15 to 36, 20 to 25, 20 to 30, 20 to 36, 25 to 30, 25 to 36, or 30 to 36. In certain embodiments, m is 0, 1, 2, 3, 4, 5, 10, 15, 20, 25, 30, or 36.

In some embodiments, R₁ is a moiety comprising a click chemistry handle. A “click chemistry handle” generally refers to a reactant, or a reactive group, that can partake in a click chemistry reaction. A “click chemistry reaction” generally refers to a reaction tailored to generate covalent bonds quickly and reliably by joining small units comprising reactive groups together. See, e.g., Kolb, Finn and Sharpless Angewandte Chemie International Edition (2001) 40: 2004-2021; Evans, Australian Journal of Chemistry (2007) 60: 384-395). Exemplary click chemistry reactions include, but are not limited to, strain-promoted azide-alkyne cycloaddition, Cu-catalyzed azide-alkyne cycloaddition, azide-alkyne Huisgen cycloaddition, Diels-Alder reaction (e.g., tetrazine [4+2] cycloaddition), and thiol-ene addition. In some embodiments, click chemistry reactions are modular, wide in scope, give high chemical yields, generate inoffensive byproducts, are stereospecific, exhibit a large thermodynamic driving force (e.g., greater than 84 kJ/mol) to favor a reaction with a single reaction product, and/or can be carried out under physiological conditions. In some embodiments, a click chemistry reaction exhibits high atom economy, can be carried out under simple reaction conditions, uses readily available starting materials and reagents, uses no toxic solvents or use a solvent that is benign or easily removed (preferably water), and/or provides simple product isolation by non-chromatographic methods (crystallization or distillation). In some embodiments, click chemistry handles are used that can react to form covalent bonds in the absence of a metal catalyst (e.g., a copper catalyst). In some embodiments, click chemistry handles are used that can react to form covalent bonds in the presence of a metal catalyst (e.g., copper (II)).

In general, click chemistry reactions require at least two molecules comprising click chemistry handles that can react with each other. Such click chemistry handle pairs that are reactive with each other are sometimes referred to herein as partner click chemistry handles or complementary click chemistry handles. For example, an azide may be a partner click chemistry handle to a strained alkyne (e.g., a cyclooctyne). Exemplary click chemistry handles suitable for use according to some aspects of this invention are described herein, for example, in Tables 1 and 2.

TABLE 1
Exemplary click chemistry handles and reactions.

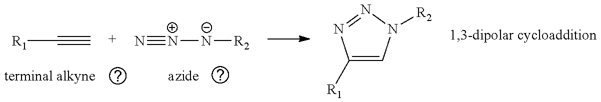

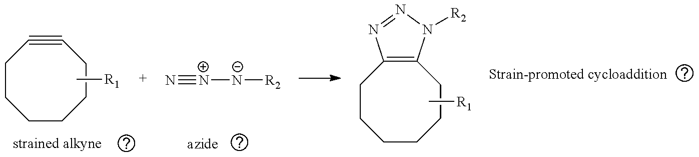

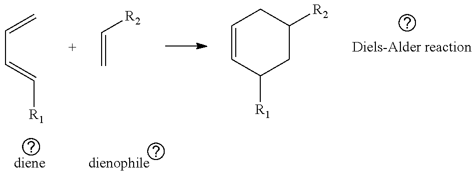

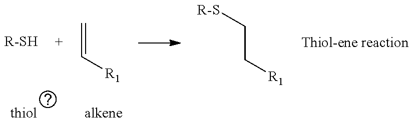

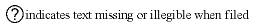

TABLE 2
Exemplary click chemistry handles and reactions (from Becer, Hoogenboom, and Schubert, Click Chemistry
Beyond Metal-Catalyzed Cycloaddition, Angewandte Chemie International Edition (2009) 48: 4900-4908.).

| | Reagent A | Reagent B | Mechanism | Notes on reaction^{[a]} | Reference |
|---|---|---|---|---|---|
| 0 | azide | alkyne | Cu-catalyzed [3 + 2] azide-alkyne | 2 h at 60° C. in H₂O | [9] |
| | | | cycloaddition (CuAAC) | | |
| 1 | azide | cyclooctyne | strain-promoted [3 + 2] azide-alkyne | 1 h at RT | [6-8, |
| | | | cycloaddition (SPAAC) | | 10, 11] |
| 2 | azide | activated alkyne | [3 + 2] Huisgen cycloaddition | 4 h at 50° C. | [12] |
| 3 | azide | electron-deficient | [3 + 2] cycloaddition | 12 h at RT in H₂O | [13] |
| | | alkyne | | | |
| 4 | azide | aryne | [3 + 2] cycloaddition | 4 h at RT in THF with crown ether or | [14, 15] |
| | | | | 24 h at RT in CH₃CN | |
| 5 | tetrazine | alkene | Diels-Alder retro-[4 + 2] cycloaddition | 40 min at 25° C. (100% yield) | [36-38] |
| | | | | N₂ is the only by-product | |
| 6 | tetrazole | alkene | 1,3-dipolar cycloaddition (photoclick) | few min UV irradiation and then | [39, 40] |
| | | | | overnight at 4° C. | |
| 7 | dithioester | diene | hetero-Diels-Alder cycloaddition | 10 min at RT | [43] |
| 8 | anthracene | maleimide | [4 + 2] Diels-Alder reaction | 2 days at reflux in toluene | [41] |
| 9 | thiol | alkene | radical addition | 30 min UV (quantitative conv.) or | [19-23] |
| | | | (thio click) | 24 h UV irradiation (>96%) | |
| 10 | thiol | enone | Michael addition | 24 h at RT in CH₃CN | [27] |
| 11 | thiol | maleimide | Michael addition | 1 h at 40° C. in THF or | [24-26] |
| | | | | 16 h at RT in dioxane | |
| 12 | thiol | para-fluoro | nucleophilic substitution | overnight at RT in DMF or | [32] |
| | | | | 60 min at 40° C. in DMF | |
| 13 | amine | para-fluoro | nucleophilic substitution | 20 min MW at 95° C. in NMP as | [30] |
| | | | | solvent | |

^{[a]}RT = room temperature, DMF = N,N-dimethylformamide, NMP = N-methylpyrolidone, THF = tetrahydrofuran, CN₃CN = acetonitrile

In some embodiments, R₁ is a moiety comprising a click chemistry handle selected from the group consisting of an azide, tetrazine, nitrile oxide, alkyne, or alkene. In some embodiments, the click chemistry handle of R₁ can participate in a strain-promoted cycloaddition reaction. In certain embodiments, the click chemistry handle of R₁ comprises an azide. In certain embodiments, the click chemistry handle of R₁ comprises a strained alkene. In some instances, the strained alkene comprises a trans-cyclooctene and/or a cyclopropene. In certain embodiments, the click chemistry handle of R₁ comprises an alkyne. In some instances, the alkyne is a strained alkyne. In some instances, the strained alkyne comprises a cyclic alkyne (e.g., a cyclooctyne). The cyclic alkyne may be a monocyclic alkyne or a polycyclic alkyne. In certain cases, the click chemistry handle of R₁ comprises dibenzoazacyclooctyne (DIBAC or DBCO), biarylazacyclooctynone (BARAC), dibenzocyclooctyne (DIBO), difluorinated cyclooctyne (DIFO), bicyclononyne (BCN), dimethoxyazacyclooctyne (DIMAC), monofluorinated cyclooctyne (MOFO), cyclooctyne (OCT), and/or aryl-less cyclooctyne (ALO). Structures of these compounds are shown below.

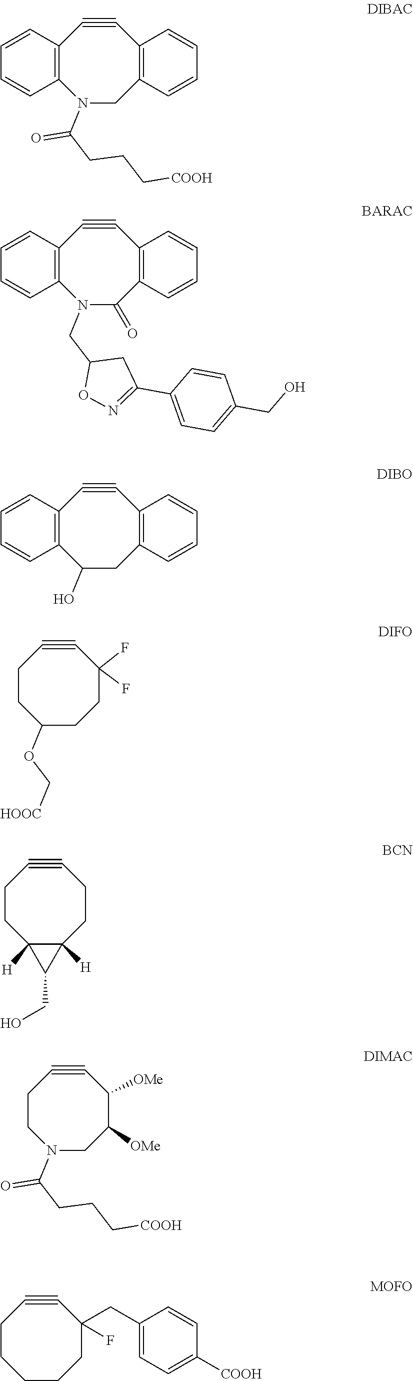

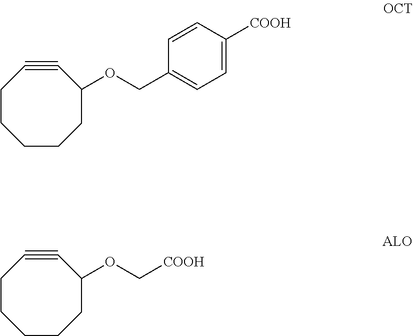 [SMILES: O=C(O)c1ccc(COC2C#CCCCCC2)cc1 ; O=C(O)COC1C#CCCCCC1]

In some embodiments, a molar ratio of R₁-(L₁)ₘ—S—X, or a salt thereof, to the polypeptide comprising at least one dehydroalanine residue, or a salt thereof, is relatively low. In some embodiments, the molar ratio of R₁-(L₁)ₘ-S—X, or a salt thereof, to the polypeptide comprising at least one dehydroalanine residue, or a salt thereof, is about 1000:1 or less, 500:1 or less, 200:1 or less, 100:1 or less, 50:1 or less, 20:1 or less, 10:1 or less, 5:1 or less, 4:1 or less, 3:1 or less, 2:1 or less, or 1.5:1 or less. In some embodiments, the molar ratio of R₁-(L₁)ₘ-S—X, or a salt thereof, to the polypeptide comprising at least one dehydroalanine residue, or a salt thereof, is in a range from 1.5:1 to 2:1, 1:5:1 to 3:1, 1.5:1 to 4:1, 1.5:1 to 5:1, 1.5:1 to 10:1, 1.5:1 to 20:1, 1.5:1 to 50:1, 1.5:1 to 100:1, 1.5:1 to 200:1, 1.5:1 to 500:1, 1.5:1 to 1000:1, 2:1 to 3:1, 2:1 to 4:1, 2:1 to 5:1, 2:1 to 10:1, 2:1 to 20:1, 2:1 to 50:1, 2:1 to 100:1, 2:1 to 200:1, 2:1 to 500:1, 2:1 to 1000:1, 5:1 to 10:1, 5:1 to 20:1, 5:1 to 50:1, 5:1 to 100:1, 5:1 to 200:1, 5:1 to 500:1, 5:1 to 1000:1, 10:1 to 20:1, 10:1 to 50:1, 10:1 to 100:1, 10:1 to 200:1, 10:1 to 500:1, 10:1 to 1000:1, 20:1 to 50:1, 20:1 to 100:1, 20:1 to 200:1, 20:1 to 500:1, 20:1 to 1000:1, 50:1 to 100:1, 50:1 to 200:1, 50:1 to 500:1, 50:1 to 1000:1, 100:1 to 200:1, 100:1 to 500:1, 100:1 to 1000:1, 200:1 to 500:1, 200:1 to 1000:1, or 500:1 to 1000:1.

The polypeptide comprising at least one dehydroalanine residue, or a salt thereof, may have any suitable length. In some embodiments, the polypeptide, or a salt thereof, has 2-10 amino acid residues, 2-25 amino acid residues, 2-50 amino acid residues, 2-75 amino acid residues, 2-100 amino acid residues, 2-200 amino acid residues, 2-500 amino acid residues, 2 1000 amino acid residues, 2-2000 amino acid residues, 10-25 amino acid residues, 10-50 amino acid residues, 10-75 amino acid residues, 10-100 amino acid residues, 10-200 amino acid residues, 10-500 amino acid residues, 10-1000 amino acid residues, 10-2000 amino acid residues, 50-100 amino acid residues, 50-200 amino acid residues, 50-500 amino acid residues, 50-1000 amino acid residues, 50-2000 amino acid residues, 100-200 amino acid residues, 100 500 amino acid residues, 100-1000 amino acid residues, 100-2000 amino acid residues, 200 500 amino acid residues, 200-1000 amino acid residues, 200-2000 amino acid residues, 500 1000 amino acid residues, 500-2000 amino acid residues, or 1000-2000 amino acid residues.

### Sample Preparation

Some aspects are directed to systems, methods, and kits for preparing samples for systems and methods for characterizing phosphoserine-containing polypeptides (e.g., identifying and/or determining the location of a phosphoserine residue in a polypeptide).

In some embodiments, a sample preparation method comprises converting a phosphoserine residue of a polypeptide, or a salt thereof, to a dehydroalanine residue to provide a dehydroalanine-containing polypeptide, or a salt thereof. In certain embodiments, the sample preparation method comprises reacting a phosphoserine-containing polypeptide, or a salt thereof, with one or more bases. In some instances, reaction of the phosphoserine-containing polypeptide, or a salt thereof, with one or more bases may result in β-elimination of the phosphate group of the phosphoserine residue to yield a dehydroalanine residue. Non-limiting example of suitable bases include barium hydroxide (Ba(OH)₂) and sodium hydroxide (NaOH).

In some embodiments, a molar ratio of the one or more bases to the phosphoserine-containing polypeptide, or a salt thereof, is relatively low. In certain embodiments, the molar ratio of the one or more bases to the phosphoserine-containing polypeptide, or a salt thereof, is about 100:1 or less, about 50:1 or less, about 20:1 or less, about 10:1 or less, about 5:1 or less, or about 2:1 or less. In certain embodiments, the molar ratio of the one or more bases to the phosphoserine-containing polypeptide, or a salt thereof, is in a range from 2:1 to 5:1, 2:1 to 10:1, 2:1 to 20:1, 2:1 to 50:1, 2:1 to 100:1, 5:1 to 10:1, 5:1 to 20:1, 5:1 to 50:1, 5:1 to 100:1, 10:1 to 20:1, 10:1 to 50:1, 10:1 to 100:1, 20:1 to 50:1, 20:1 to 100:1, or 50:1 to 100:1.

In some embodiments, the reaction of the phosphoserine-containing polypeptide, or a salt thereof, with one or more bases is performed for a relatively short amount of time. In certain embodiments, the reaction of the phosphoserine-containing polypeptide, or a salt thereof, with one or more bases has a reaction time of about 120 minutes or less, 60 minutes or less, 45 minutes or less, 30 minutes or less, 25 minutes or less, 20 minutes or less, 15 minutes or less, 10 minutes or less, or 5 minutes or less. In certain embodiments, the reaction of the phosphoserine-containing polypeptide, or a salt thereof, with one or more bases has a reaction time in a range from 5 to 10 minutes, 5 to 15 minutes, 5 to 20 minutes, 5 to 25 minutes, 5 to 30 minutes, 5 to 45 minutes, 5 to 60 minutes, 5 to 120 minutes, 10 to 30 minutes, 10 to 45 minutes, 10 to 60 minutes, 10 to 120 minutes, 30 minutes to 45 minutes, 30 minutes to 60 minutes, 30 to 120 minutes, or 60 to 120 minutes.

The reaction of the phosphoserine-containing polypeptide, or a salt thereof, with one or more bases may be performed at various temperatures. In certain embodiments, the reaction of the phosphoserine-containing polypeptide, or a salt thereof, with one or more bases is performed at a reaction temperature in a range from 20° C. to 25° C., 20° C. to 30° C., 20° C. to 37° C., 20° C. to 40° C., 20° C. to 45° C., 20° C. to 50° C., 20° C. to 55° C., 20° C. to 60° C., 25° C. to 30° C., 25° C. to 37° C., 25° C. to 40° C., 25° C. to 45° C., 25° C. to 50° C., 25° C. to 55° C., 25° C. to 60° C., 30° C. to 37° C., 30° C. to 40° C., 30° C. to 45° C., 30° C. to 50° C., 30° C. to 55° C., 30° C. to 60° C., 37° C. to 45° C., 37° C. to 50° C., 37° C. to 55° C., 37° C. to 60° C., 40° C. to 50° C., 40° C. to 55° C., 40° C. to 60° C., or 50° C. to 60° C. In certain embodiments, the reaction of the phosphoserine-containing polypeptide, or a salt thereof, with one or more bases is performed at a reaction temperature of about 20° C., 25° C., 30° C., 35° C., 37° C., 40° C., 45° C., 50° C., 55° C., or 60° C. In certain embodiments, the reaction temperature is about 37° C.

In some embodiments, the sample preparation method comprises reacting the dehydroalanine-containing polypeptide, or a salt thereof, with a protected thiol compound of Formula (I):

R₁-(L₁)ₘ-S—X (I),

or a salt thereof, to provide a functionalized polypeptide comprising a residue of Formula (II):

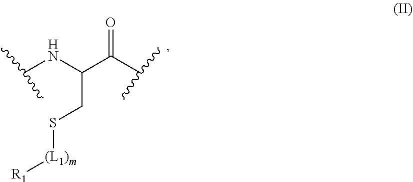

or a salt thereof, where X, L₁, m, and R₁ are described herein.

In some embodiments, the sample preparation method comprises digesting the functionalized polypeptide, or a salt thereof, to form two or more peptide fragments. In some embodiments, at least one peptide fragment of the two or more peptide fragments is a functionalized peptide fragment comprising R₁.

In some embodiments, digesting the functionalized polypeptide comprises exposing the functionalized polypeptide to a polypeptide-cleaving agent. In certain embodiments, the polypeptide-cleaving agent comprises one or more enzymes. In some cases, the one or more enzymes comprise one or more proteases. Examples of suitable proteases include, but are not limited to, Arg-C (also referred to as clostripain), Lys-C, Glu-C, trypsin, chymotrypsin, Asp-N, Lys-N, Asp-X, thrombin, elastase, subtilisin, pepsin, thermolysin, proteinase K, carboxypeptidase A, carboxypeptidase B, cathepsin C, caspase, glutamyl endopeptidase, and proline endopeptidase. In some embodiments, the protease comprises Arg-C. In some embodiments, the protease comprises Lys-C. In certain embodiments, the polypeptide-cleaving agent comprises one or more non-enzymatic reagents. Examples of suitable non-enzymatic reagents include, but are not limited to, cyanogen bromide, formic acid, hydrochloric acid, hydroxylamine, 2-nitro-5-thiocyanatobenzoic acid, BNPS-skatole, and iodosobenzoic acid.

In some embodiments, a sample preparation method comprises digesting a polypeptide comprising a phosphoserine residue, or a salt thereof, to form two or more peptide fragments, or a salt thereof. In some embodiments, at least one peptide fragment of the two or more peptide fragments, or a salt thereof, comprises the phosphoserine residue.

In some embodiments, digesting the polypeptide comprising the phosphoserine residue, or a salt thereof, comprises exposing the polypeptide comprising the phosphoserine residue, or a salt thereof, to a polypeptide-cleaving agent. In certain embodiments, the polypeptide-cleaving agent comprises one or more enzymes. In some cases, the one or more enzymes comprise one or more proteases. Examples of suitable proteases include, but are not limited to, Arg-C (also referred to as clostripain), Lys-C, Glu-C, trypsin, chymotrypsin, Asp-N, Lys-N, Asp-X, thrombin, elastase, subtilisin, pepsin, thermolysin, proteinase K, carboxypeptidase A, carboxypeptidase B, cathepsin C, caspase, glutamyl endopeptidase, and proline endopeptidase. In some embodiments, the protease comprises Arg-C. In some embodiments, the protease comprises Lys-C. In certain embodiments, the polypeptide-cleaving agent comprises one or more non-enzymatic reagents. Examples of suitable non-enzymatic reagents include, but are not limited to, cyanogen bromide, formic acid, hydrochloric acid, hydroxylamine, 2-nitro-5-thiocyanatobenzoic acid, BNPS-skatole, and iodosobenzoic acid.

In some embodiments, the sample preparation method comprises converting the phosphoserine residue of the at least one peptide fragment, or a salt thereof, to a dehydroalanine residue of the at least one peptide fragment, or a salt thereof, to provide a dehydroalanine-containing peptide fragment, or a salt thereof. In certain embodiments, the sample preparation method comprises reacting a phosphoserine-containing peptide fragment, or a salt thereof, with one or more bases. In some instances, reaction of the phosphoserine-containing peptide fragment, or a salt thereof, with one or more bases may result in β-elimination of the phosphate group of the phosphoserine residue to yield a dehydroalanine residue. Non-limiting example of suitable bases include barium hydroxide (Ba(OH)₂) and sodium hydroxide (NaOH).

In some embodiments, a molar ratio of the one or more bases to the phosphoserine-containing peptide fragment, or a salt thereof, is relatively low. In certain embodiments, the molar ratio of the one or more bases to the phosphoserine-containing peptide fragment, or a salt thereof, is about 100:1 or less, about 50:1 or less, about 20:1 or less, about 10:1 or less, about 5:1 or less, or about 2:1 or less. In certain embodiments, the molar ratio of the one or more bases to the phosphoserine-containing peptide fragment, or a salt thereof, is in a range from 2:1 to 5:1, 2:1 to 10:1, 2:1 to 20:1, 2:1 to 50:1, 2:1 to 100:1, 5:1 to 10:1, 5:1 to 20:1, 5:1 to 50:1, 5:1 to 100:1, 10:1 to 20:1, 10:1 to 50:1, 10:1 to 100:1, 20:1 to 50:1, 20:1 to 100:1, or 50:1 to 100:1.

In some embodiments, the reaction of the phosphoserine-containing peptide fragment, or a salt thereof, with one or more bases is performed for a relatively short amount of time. In certain embodiments, the reaction of the phosphoserine-containing peptide fragment, or a salt thereof, with one or more bases has a reaction time of about 120 minutes or less, 60 minutes or less, 45 minutes or less, 30 minutes or less, 25 minutes or less, 20 minutes or less, 15 minutes or less, 10 minutes or less, or 5 minutes or less. In certain embodiments, the reaction of the phosphoserine-containing peptide fragment, or a salt thereof, with one or more bases has a reaction time in a range from 5 to 10 minutes, 5 to 15 minutes, 5 to 20 minutes, 5 to 25 minutes, 5 to 30 minutes, 5 to 45 minutes, 5 to 60 minutes, 5 to 120 minutes, 10 to 30 minutes, 10 to 45 minutes, 10 to 60 minutes, 10 to 120 minutes, 30 minutes to 45 minutes, 30 minutes to 60 minutes, 30 to 120 minutes, or 60 to 120 minutes.

The reaction of the phosphoserine-containing peptide fragment, or a salt thereof, with one or more bases may be performed at various temperatures. In certain embodiments, the reaction of the phosphoserine-containing peptide fragment, or a salt thereof, with one or more bases is performed at a reaction temperature in a range from 20° C. to 25° C., 20° C. to 30° C., 20° C. to 37° C., 20° C. to 40° C., 20° C. to 45° C., 20° C. to 50° C., 20° C. to 55° C., 20° C. to 60° C., 25° C. to 30° C., 25° C. to 37° C., 25° C. to 40° C., 25° C. to 45° C., 25° C. to 50° C., 25° C. to 55° C., 25° C. to 60° C., 30° C. to 37° C., 30° C. to 40° C., 30° C. to 45° C., 30° C. to 50° C., 30° C. to 55° C., 30° C. to 60° C., 37° C. to 45° C., 37° C. to 50° C., 37° C. to 55° C., 37° C. to 60° C., 40° C. to 50° C., 40° C. to 55° C., 40° C. to 60° C., or 50° C. to 60° C. In certain embodiments, the reaction of the phosphoserine-containing peptide fragment, or a salt thereof, with one or more bases is performed at a reaction temperature of about 20° C., 25° C., 30° C., 35° C., 37° C., 40° C., 45° C., 50° C., 55° C., or 60° C. In certain embodiments, the reaction temperature is about 37° C.

In some embodiments, the sample preparation method comprises reacting the dehydroalanine-containing peptide fragment, or a salt thereof, with a protected thiol compound of Formula (I):

R₁-(L₁)ₘ-S—X (I),

or a salt thereof, to provide a functionalized peptide fragment comprising a residue of Formula (II),

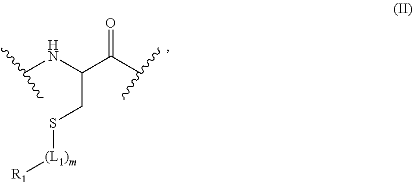

or a salt thereof, where X, L₁, m, and R₁ are described herein.

In some embodiments, the sample preparation method comprises conjugating the functionalized peptide fragment to a linker. In certain embodiments, the linker is a compound of Formula (III):

R₂-L₂-B (III)
wherein:
   R₂ is a moiety comprising a click chemistry handle;
   L₂ is a bond or a linking group; and
   B is a binding group.

In some embodiments, R₂ is a moiety comprising a click chemistry handle that is complementary (e.g., a partner click chemistry handle) to R₁ of the protected thiol compound (e.g., a compound of Formula (I) or a salt thereof) and the functionalized peptide (e.g., a peptide having a residue of Formula (II) or a salt thereof). In some embodiments, the click chemistry handle of R₂ is capable of undergoing a click chemistry reaction with the click chemistry handle of R₁. For example, when R₁ comprises an azide, nitrile oxide, or tetrazine, then R₂ may comprise an alkyne or strained alkene. Conversely, when R₁ comprises an alkyne or strained alkene, then R₂ may comprise an azide, nitrile oxide, or tetrazine.

In some embodiments, R₂ is a moiety comprising a click chemistry handle selected from the group consisting of an azide, tetrazine, nitrile oxide, alkyne, or alkene. In some embodiments, the click chemistry handle of R₂ can participate in a strain-promoted cycloaddition reaction. In certain embodiments, the click chemistry handle of R₂ comprises an alkyne. In some instances, the alkyne is a strained alkyne. In some instances, the strained alkyne comprises a cyclic alkyne (e.g., a cyclooctyne). The cyclic alkyne may be a monocyclic alkyne or a polycyclic alkyne. In certain cases, the click chemistry handle of R₂ comprises dibenzoazacyclooctyne (DIBAC or DBCO), biarylazacyclooctynone (BARAC), dibenzocyclooctyne (DIBO), difluorinated cyclooctyne (DIFO), bicyclononyne (BCN), dimethoxyazacyclooctyne (DIMAC), monofluorinated cyclooctyne (MOFO), cyclooctyne (OCT), and/or aryl-less cyclooctyne (ALO). In certain embodiments, the click chemistry handle of R₂ comprises a strained alkene. In some instances, the strained alkene comprises a trans-cyclooctene and/or a cyclopropene. In certain embodiments, the click chemistry handle of R₂ comprises an azide.

In certain embodiments, the click chemistry handle of R₁ and/or R₂ is of Formula (IV) or Formula (V):

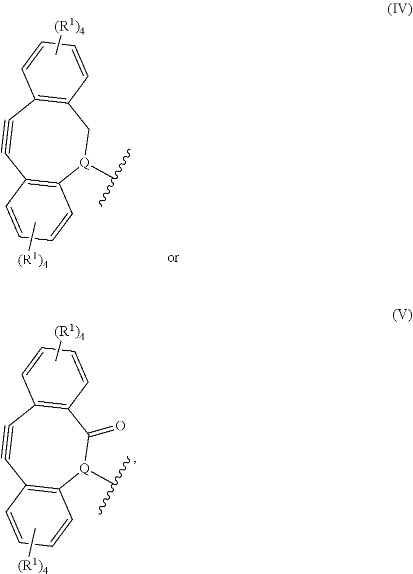

or a salt thereof, wherein:
each instance of R¹ is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, -Osi(R^{A})₃, -Osi(R^{A})₂OR^{A}, -Osi(R^{A})(OR^{A})₂, -Osi(OR^{A})₃, or
each occurrence of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{A} are joined together with their intervening atom to form an optionally substituted heterocyclic ring or optionally substituted heteroaryl ring; and
Q is CH or N.

In certain embodiments, the click chemistry handle is of formula (VI):

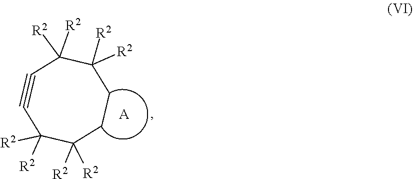

or a salt thereof, wherein:
each instance of R² is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, -Osi(R^{A})₃, -Osi(R^{A})₂OR^{A}, -Osi(R^{A})(OR^{A})₂, -Osi(OR^{A})₃, or or two instances of R² attached to the same carbon atom are taken together to form ═O or ═S;
each occurrence of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{A} are joined together with their intervening atom to form an optionally substituted heterocyclic ring or optionally substituted heteroaryl ring; and
Ring A is optionally substituted carbocyclyl, optionally substituted heterocyclyl, optionally substituted aryl, or optionally substituted heteroaryl.

In certain embodiments, the click chemistry handle is of formula (VI-a):

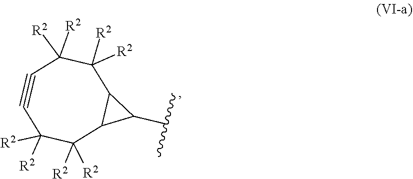

or a salt thereof, wherein: (VI-a),
each instance of R² is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, -Osi(R^{A})₃, -Osi(R^{A})₂OR^{A}, -Osi(R^{A})(OR^{A})₂, -Osi(OR^{A})₃, or and
each occurrence of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{A} are joined together with their intervening atom to form an optionally substituted heterocyclic ring or optionally substituted heteroaryl ring.

In certain embodiments, the click chemistry handle is of Formulae (VII-a), (VII-b), (VII-c), or (VII-d):

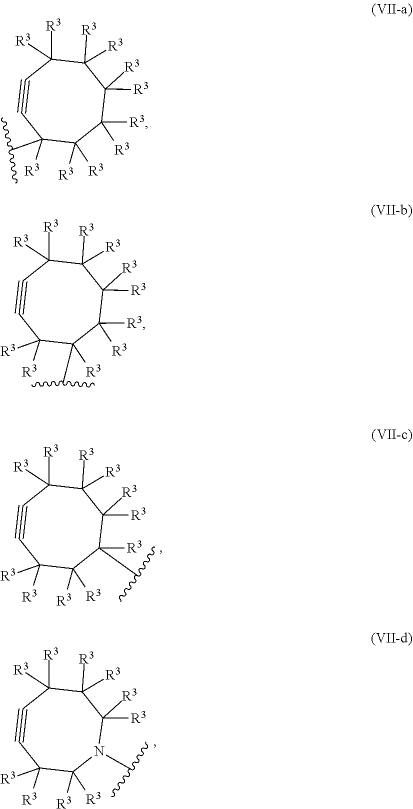

or a salt thereof, wherein:
each instance of R³ is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, -Osi(R^{A})₃, -Osi(R^{A})₂OR^{A}, -Osi(R^{A})(OR^{A})₂, -Osi(OR^{A})₃, or or two instances of R³ attached to the same carbon atom are taken together to form ═O or ═S; and
each occurrence of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{A} are joined together with their intervening atom to form an optionally substituted heterocyclic ring or optionally substituted heteroaryl ring.

In some embodiments, L₂ comprises a polypeptidyl group. In certain embodiments, the polypeptidyl group of L₂ comprises at least 5 amino acid residues, at least 10 amino acid residues, at least 15 amino acid residues, or at least 20 amino acid residues. In certain embodiments, the polypeptidyl group of L₂ comprises between 5 and 10 amino acid residues, between 5 and 15 amino acid residues, between 5 and 20 amino acid residues, between 10 and 15 amino acid residues, between 10 and 20 amino acid residues, or between 15 and 20 amino acid residues.

In some embodiments, the polypeptidyl group of L₂ has a length of at least about 20 Å, 25 Å, 30 Å, 35 Å, 40 Å, 45 Å, 50 Å, 55 Å, 60 Å, 65 Å, 70 Å, or 75 Å. In certain embodiments, the polypeptidyl group of L₂ has a length in a range from 20 Å to 30 Å, 20 Å to 35 Å, 20 Å to 40 Å, 20 Å to 45 Å, 20 Å to 50 Å, 20 Å to 55 Å, 20 Å to 60 Å, 20 Å to 65 Å, 20 Å to 70 Å, 20 Å to 75 Å, 30 Å to 40 Å, 30 Å to 45 Å, 30 Å to 50 Å, 30 Å to 55 Å, 30 Å to 60 Å, 30 Å to 65 Å, 30 Å to 70 Å, 30 Å to 75 Å, 40 Å to 50 Å, 40 Å to 55 Å, 40 Å to 60 Å, 40 Å to 65 Å, 40 Å to 70 Å, 40 Å to 75 Å, 50 Å to 60 Å, 50 Å to 65 Å, 50 Å to 70 Å, 50 Å to 75 Å, 60 Å to 70 Å, or 60 Å to 75 Å. In certain embodiments, the sulfur atom and R₁ of L₂ are separated by a minimum distance of at least about 20 A, 25 Å, 30 Å, 35 Å, 40 Å, 45 Å, 50 Å, 55 Å, 60 Å, 65 Å, 70 Å, or 75 Å. In certain embodiments, the sulfur atom and R₁ of L₂ are separated by a minimum distance in a range from 20 Å to 30 Å, 20 Å to 35 Å, 20 Å to 40 Å, 20 Å to 45 Å, 20 Å to 50 Å, 20 Å to 55 Å, 20 Å to 60 Å, 20 Å to 65 Å, 20 Å to 70 Å, 20 Å to 75 Å, 30 Å to 40 Å, 30 Å to 45 Å, 30 Å to 50 Å, 30 Å to 55 Å, 30 Å to 60 Å, 30 Å to 65 Å, 30 Å to 70 Å, 30 Å to 75 Å, 40 Å to 50 Å, 40 Å to 55 Å, 40 Å to 60 Å, 40 Å to 65 Å, 40 Å to 70 Å, 40 Å to 75 Å, 50 Å to 60 Å, 50 Å to 65 Å, 50 Å to 70 Å, 50 Å to 75 Å, 60 Å to 70 Å, or 60 Å to 75 Å.

In some embodiments, the polypeptidyl group of L₂ comprises at least 1 negatively charged moiety at physiological pH. In certain embodiments, the polypeptidyl group of L₂ comprises at least 1, at least 2, at least 3, at least 4, at least 5, at least 6, at least 7, at least 8, at least 9, at least 10, at least 11, at least 12, at least 13, at least 14, or at least 15 negatively charged moieties at physiological pH. In certain embodiments, the polypeptidyl group of L₂ comprises between 1 and 2, 1 and 3, 1 and 4, 1 and 5, 1 and 6, 1 and 7, 1 and 8, 1 and 9, 1 and 10, 1 and 11, 1 and 12, 1 and 13, 1 and 14, 1 and 15, 2 and 3, 2 and 4, 2 and 5, 2 and 6, 2 and 7, 2 and 8, 2 and 9, 2 and 10, 2 and 11, 2 and 12, 2 and 13, 2 and 14, 2 and 15, 3 and 4, 3 and 5, 3 and 6, 3 and 7, 3 and 8, 3 and 9, 3 and 10, 3 and 11, 3 and 12, 3 and 13, 3 and 14, 3 and 15, 4 and 5, 4 and 6, 4 and 7, 4 and 8, 4 and 9, 4 and 10, 4 and 11, 4 and 12, 4 and 13, 4 and 14, 4 and 15, 5 and 6, 5 and 7, 5 and 8, 5 and 9, 5 and 10, 5 and 11, 5 and 12, 5 and 13, 5 and 14, 5 and 15, 6 and 10, 6 and 15, 7 and 10, 7 and 15, 8 and 10, 8 and 15, 9 and 10, 9 and 15, or 10 and 15 negatively charged moieties at physiological pH.

In some embodiments, the polypeptidyl group of L₂ comprises at least 1 aspartate residue. In certain embodiments, the polypeptidyl group of L₂ comprises at least 1, at least 2, at least 3, at least 4, at least 5, at least 6, at least 7, at least 8, at least 9, at least 10, at least 11, at least 12, at least 13, at least 14, or at least 15 aspartate residues. In certain embodiments, the polypeptidyl group of L₂ comprises between 1 and 2, 1 and 3, 1 and 4, 1 and 5, 1 and 6, 1 and 7, 1 and 8, 1 and 9, 1 and 10, 1 and 11, 1 and 12, 1 and 13, 1 and 14, 1 and 15, 2 and 3, 2 and 4, 2 and 5, 2 and 6, 2 and 7, 2 and 8, 2 and 9, 2 and 10, 2 and 11, 2 and 12, 2 and 13, 2 and 14, 2 and 15, 3 and 4, 3 and 5, 3 and 6, 3 and 7, 3 and 8, 3 and 9, 3 and 10, 3 and 11, 3 and 12, 3 and 13, 3 and 14, 3 and 15, 4 and 5, 4 and 6, 4 and 7, 4 and 8, 4 and 9, 4 and 10, 4 and 11, 4 and 12, 4 and 13, 4 and 14, 4 and 15, 5 and 6, 5 and 7, 5 and 8, 5 and 9, 5 and 10, 5 and 11, 5 and 12, 5 and 13, 5 and 14, 5 and 15, 6 and 10, 6 and 15, 7 and 10, 7 and 15, 8 and 10, 8 and 15, 9 and 10, 9 and 15, or 10 and 15 aspartate residues.

In some embodiments, the polypeptidyl group of L₂ comprises at least 1 phenylalanine residue. In certain embodiments, the polypeptidyl group of L₂ comprises at least 1, at least 2, at least 3, at least 4, at least 5, at least 6, at least 7, at least 8, at least 9, at least 10, at least 11, at least 12, at least 13, at least 14, or at least 15 phenylalanine residues. In certain embodiments, the polypeptidyl group of L₂ comprises between 1 and 2, 1 and 3, 1 and 4, 1 and 5, 1 and 6, 1 and 7, 1 and 8, 1 and 9, 1 and 10, 1 and 11, 1 and 12, 1 and 13, 1 and 14, 1 and 15, 2 and 3, 2 and 4, 2 and 5, 2 and 6, 2 and 7, 2 and 8, 2 and 9, 2 and 10, 2 and 11, 2 and 12, 2 and 13, 2 and 14, 2 and 15, 3 and 4, 3 and 5, 3 and 6, 3 and 7, 3 and 8, 3 and 9, 3 and 10, 3 and 11, 3 and 12, 3 and 13, 3 and 14, 3 and 15, 4 and 5, 4 and 6, 4 and 7, 4 and 8, 4 and 9, 4 and 10, 4 and 11, 4 and 12, 4 and 13, 4 and 14, 4 and 15, 5 and 6, 5 and 7, 5 and 8, 5 and 9, 5 and 10, 5 and 11, 5 and 12, 5 and 13, 5 and 14, 5 and 15, 6 and 10, 6 and 15, 7 and 10, 7 and 15, 8 and 10, 8 and 15, 9 and 10, 9 and 15, or 10 and 15 phenylalanine residues.

In some embodiments, the polypeptidyl group of L₂ comprises at least 1 glycine residue. In certain embodiments, the polypeptidyl group of L₂ comprises at least 1, at least 2, at least 3, at least 4, at least 5, at least 6, at least 7, at least 8, at least 9, at least 10, at least 11, at least 12, at least 13, at least 14, or at least 15 glycine residues. In certain embodiments, the polypeptidyl group of L₂ comprises between 1 and 2, 1 and 3, 1 and 4, 1 and 5, 1 and 6, 1 and 7, 1 and 8, 1 and 9, 1 and 10, 1 and 11, 1 and 12, 1 and 13, 1 and 14, 1 and 15, 2 and 3, 2 and 4, 2 and 5, 2 and 6, 2 and 7, 2 and 8, 2 and 9, 2 and 10, 2 and 11, 2 and 12, 2 and 13, 2 and 14, 2 and 15, 3 and 4, 3 and 5, 3 and 6, 3 and 7, 3 and 8, 3 and 9, 3 and 10, 3 and 11, 3 and 12, 3 and 13, 3 and 14, 3 and 15, 4 and 5, 4 and 6, 4 and 7, 4 and 8, 4 and 9, 4 and 10, 4 and 11, 4 and 12, 4 and 13, 4 and 14, 4 and 15, 5 and 6, 5 and 7, 5 and 8, 5 and 9, 5 and 10, 5 and 11, 5 and 12, 5 and 13, 5 and 14, 5 and 15, 6 and 10, 6 and 15, 7 and 10, 7 and 15, 8 and 10, 8 and 15, 9 and 10, 9 and 15, or 10 and 15 glycine residues.

In some embodiments, the polypeptidyl group of L₂ comprises at least 1 proline residue. In certain embodiments, the polypeptidyl group of L₂ comprises at least 1, at least 2, at least 3, at least 4, at least 5, at least 6, at least 7, at least 8, at least 9, at least 10, at least 11, at least 12, at least 13, at least 14, or at least 15 proline residues. In certain embodiments, the polypeptidyl group of L₂ comprises between 1 and 2, 1 and 3, 1 and 4, 1 and 5, 1 and 6, 1 and 7, 1 and 8, 1 and 9, 1 and 10, 1 and 11, 1 and 12, 1 and 13, 1 and 14, 1 and 15, 2 and 3, 2 and 4, 2 and 5, 2 and 6, 2 and 7, 2 and 8, 2 and 9, 2 and 10, 2 and 11, 2 and 12, 2 and 13, 2 and 14, 2 and 15, 3 and 4, 3 and 5, 3 and 6, 3 and 7, 3 and 8, 3 and 9, 3 and 10, 3 and 11, 3 and 12, 3 and 13, 3 and 14, 3 and 15, 4 and 5, 4 and 6, 4 and 7, 4 and 8, 4 and 9, 4 and 10, 4 and 11, 4 and 12, 4 and 13, 4 and 14, 4 and 15, 5 and 6, 5 and 7, 5 and 8, 5 and 9, 5 and 10, 5 and 11, 5 and 12, 5 and 13, 5 and 14, 5 and 15, 6 and 10, 6 and 15, 7 and 10, 7 and 15, 8 and 10, 8 and 15, 9 and 10, 9 and 15, or 10 and 15 proline residues.

In some embodiments, the polypeptidyl group of L₂ comprises at least 1 DD repeat, GG repeat, FF repeat, DDD repeat, GGG, and/or FFF repeat. In certain embodiments, the polypeptidyl group of L₂ comprises at least 1, at least 2, at least 3, at least 4, at least 5, at least 6, at least 7, at least 8, at least 9, at least 10, at least 11, at least 12, at least 13, at least 14, or at least 15 DD repeats, GG repeats, FF repeats, DDD repeats, GGG, and/or FFF repeats. In certain embodiments, the polypeptidyl group of L₂ comprises between 1 and 2, 1 and 3, 1 and 4, 1 and 5, 1 and 6, 1 and 7, 1 and 8, 1 and 9, 1 and 10, 1 and 11, 1 and 12, 1 and 13, 1 and 14, 1 and 15, 2 and 3, 2 and 4, 2 and 5, 2 and 6, 2 and 7, 2 and 8, 2 and 9, 2 and 10, 2 and 11, 2 and 12, 2 and 13, 2 and 14, 2 and 15, 3 and 4, 3 and 5, 3 and 6, 3 and 7, 3 and 8, 3 and 9, 3 and 10, 3 and 11, 3 and 12, 3 and 13, 3 and 14, 3 and 15, 4 and 5, 4 and 6, 4 and 7, 4 and 8, 4 and 9, 4 and 10, 4 and 11, 4 and 12, 4 and 13, 4 and 14, 4 and 15, 5 and 6, 5 and 7, 5 and 8, 5 and 9, 5 and 10, 5 and 11, 5 and 12, 5 and 13, 5 and 14, 5 and 15, 6 and 10, 6 and 15, 7 and 10, 7 and 15, 8 and 10, 8 and 15, 9 and 10, 9 and 15, or 10 and 15DD repeats, GG repeats, FF repeats, DDD repeats, GGG, and/or FFF repeats.

In some embodiments, the polypeptidyl group of L₂ comprises a sequence selected from the group consisting of GPPPPPPPPG (SEQ ID NO: 3), isoEGWRW (SEQ ID NO: 4), DDGGGDDDFF (SEQ ID NO: 2), GGSSSGSGNDEEFQ (SEQ ID NO: 5), GGGGGDPDPDFF (SEQ ID NO: 6), GDGDGDGDGDFF (SEQ ID NO: 7), NNGGGNNNFF (SEQ ID NO: 8), and DDGGGCyCyCyFF (SEQ ID NO: 9), or a salt thereof, wherein Cy is a cysteic acid.

In some embodiments, L₂ comprises an oligonucleotide. In certain embodiments, the oligonucleotide of L₂ is a single-stranded oligonucleotide. In certain embodiments, the oligonucleotide of L₂ is a double-stranded oligonucleotide. In certain embodiments, the oligonucleotide of L₂ has a length of at least 15, at least 20, at least 25, at least 30, at least 35, at least 40, at least 45, or at least 50 nucleotides. In certain embodiments, the oligonucleotide of L₂ has a length in a range from 15 to 20, 15 to 25, 15 to 30, 15 to 35, 15 to 40, 15 to 45, 15 to 50, 20 to 25, 20 to 30, 20 to 35, 20 to 40, 20 to 45, 20 to 50, 25 to 30, 25 to 35, 25 to 40, 25 to 45, 25 to 50, 30 to 35, 30 to 40, 30 to 45, 30 to 50, 35 to 40, 35 to 45, 35 to 50, 40 to 45, 40 to 50, or 45 to 50 nucleotides. In certain embodiments, at least one strand of the oligonucleotide of L₂ has a sequence that is at least 50%, at least 55%, at least 60%, at least 65%, at least 70%, at least 75%, at least 80%, at least 85%, at least 90%, at least 95%, at least 99%, or 100% identical to 5′-CCACGCGTGGAACCCTTGGGATCCA-3′ (SEQ ID NO: 1). In certain embodiments, at least one strand of the oligonucleotide of L₂ has a sequence that is at least 50%, at least 55%, at least 60%, at least 65%, at least 70%, at least 75%, at least 80%, at least 85%, at least 90%, at least 95%, at least 99%, or 100% identical to 5′-TGG AGT CAA GGT CCT CTG ATG CCA T-3′ (SEQ ID NO: 27).

In some embodiments, L₂ further comprises at least one of optionally substituted alkylene, optionally substituted alkenylene, optionally substituted alkynylene, optionally substituted heteroalkylene, optionally substituted heteroalkenylene, optionally substituted heteroalkynylene, optionally substituted heterocyclylene, optionally substituted carbocyclylene, optionally substituted arylene, optionally substituted heteroarylene, or a combination thereof.

In some embodiments, the binding group B of the linker (e.g., a compound of Formula (III)) comprises at least one biotin moiety. In certain embodiments, the at least one biotin moiety comprises a bis-biotin moiety.

In some embodiments, the binding group B of the linker (e.g., a compound of Formula (III)) comprises at least one tag sequence. In certain embodiments, the at least one tag sequence comprises at least one biotin ligase recognition sequence that permits biotinylation of the linker (e.g., incorporation of one or more biotin moieties, including biotin and bis-biotin moieties). In certain embodiments, the at least one tag sequence comprises two biotin ligase recognition sequences oriented in tandem. In some cases, a biotin ligase recognition sequence refers to an amino acid sequence that is recognized by a biotin ligase, which catalyzes a covalent linkage between the sequence and a biotin molecule. Each biotin ligase recognition sequence of a tag sequence can be covalently linked to a biotin moiety, such that a tag sequence having multiple biotin ligase recognition sequences can be covalently linked to multiple biotin molecules. A region of a tag sequence having one or more biotin ligase recognition sequences can be generally referred to as a biotinylation tag or a biotinylation sequence. In some embodiments, a bis-biotin or bis-biotin moiety can refer to two biotins bound to two biotin ligase recognition sequences oriented in tandem. In certain embodiments, the binding group B of the linker (e.g., a compound of Formula (III)) comprises at least one biotin ligase recognition sequence having a biotin moiety attached thereto or at least two biotin ligase recognition sequences, each having a biotin moiety attached thereto.

In some embodiments, the binding group B of the linker (e.g., a compound of Formula (III)) comprises or is conjugated to an avidin protein. The term “avidin protein” refers to a biotin-binding protein, generally having a biotin binding site at each of four subunits of the avidin protein. Non-limiting examples of avidin proteins include avidin, streptavidin, traptavidin, tamavidin, bradavidin, xenavidin, and homologs and variants thereof. In some cases, the avidin protein may have a monomeric, dimeric, or tetrameric form. In certain embodiments, the avidin protein is streptavidin in a tetrameric form (e.g., a homotetramer). In certain embodiments, the streptavidin in a tetrameric form may be bound to one component (e.g., a first component comprising a first mono-biotin moiety or a first bis-biotin moiety), two components (e.g., a first component comprising a first mono-biotin moiety or a first bis-biotin moiety and a second component comprising a second mono-biotin moiety or a second bis-biotin moiety), three components (e.g., a first component comprising a first bis-biotin moiety, a second component comprising a first mono-biotin moiety, and a third component comprising a second mono-biotin moiety), or four components (e.g., four components, each comprising a mono-biotin moiety).

In some embodiments, the binding group B of the linker (e.g., a compound of Formula (III)) comprises a click chemistry handle. In certain embodiments, the binding group B of the linker comprises a click chemistry handle selected from the group consisting of an azide, tetrazine, nitrile oxide, alkyne, or alkene. In some embodiments, the click chemistry handle of the binding group B of the linker can participate in a strain-promoted cycloaddition reaction. In certain embodiments, the click chemistry handle of the binding group B of the linker comprises an azide. In certain embodiments, the click chemistry handle of the binding group B of the linker comprises an alkyne. In some instances, the alkyne is a strained alkyne. In some instances, the strained alkyne comprises a cyclic alkyne (e.g., a cyclooctyne). The cyclic alkyne may be a monocyclic alkyne or a polycyclic alkyne. In certain cases, the click chemistry handle of the binding group B of the linker comprises dibenzoazacyclooctyne (DIBAC or DBCO), biarylazacyclooctynone (BARAC), dibenzocyclooctyne (DIBO), difluorinated cyclooctyne (DIFO), bicyclononyne (BCN), dimethoxyazacyclooctyne (DIMAC), monofluorinated cyclooctyne (MOFO), cyclooctyne (OCT), and/or aryl-less cyclooctyne (ALO). In certain embodiments, the click chemistry handle of the binding group B of the linker comprises a strained alkene. In some instances, the strained alkene comprises a trans-cyclooctene and/or a cyclopropene.

In some embodiments, the sample preparation method comprises conjugating the functionalized peptide fragment to a linker (e.g., a compound of Formula (III)). In certain embodiments, conjugating the functionalized peptide fragment to the linker comprises performing a click chemistry reaction between the R₁ group of the functionalized peptide fragment and the R₂ group of the linker. As a non-limiting example, when R₁ comprises an azide and R₂ comprises a strained alkyne (e.g., a cyclooctyne), R₁ may react with R₂ in a strain-promoted cycloaddition reaction. Various reaction conditions (e.g., reaction times, reaction temperatures, solvents) are suitable for such strain-promoted cycloaddition reactions and other click chemistry reactions, and those of ordinary skill in the art will be able to determine appropriate reaction conditions.

In some embodiments, the sample preparation method comprises immobilizing a linker (e.g., a compound of Formula (III) conjugated to a functionalized peptide fragment) to a surface (e.g., a surface of a sample well). In certain embodiments, the surface comprises a binding moiety. In some instances, the binding moiety comprises at least one biotin moiety (e.g., a bis-biotin moiety). In some instances, the binding moiety comprises at least one avidin protein (e.g., a streptavidin). In some instances, the binding moiety comprises a click chemistry handle as described herein (e.g., an azide, tetrazine, nitrile oxide, alkyne, or alkene).

In some embodiments, the binding moiety of the surface is configured to covalently or non-covalently bind, directly or indirectly, to the binding group B of the linker (e.g., a compound of Formula (III) conjugated to a functionalized peptide fragment). In certain embodiments, the binding group B of the linker may comprise at least one biotin moiety (e.g., a bis-biotin moiety), and the binding moiety of the surface may comprise at least one biotin moiety (e.g., a bis-biotin moiety) and/or an avidin protein (e.g., a streptavidin). In some such embodiments, the at least one biotin moiety of the binding moiety of the surface may be conjugated to one or more biotin binding sites of an avidin protein, and the at least one biotin moiety of the binding group B of the linker may be conjugated to one or more biotin binding sites of the same avidin protein. In certain embodiments, the binding group B of the linker may comprise at least one biotin moiety (e.g., a bis-biotin moiety), and the binding moiety of the surface may comprise an avidin protein (e.g., a streptavidin). In some such embodiments, the at least one biotin moiety of the binding group B of the linker may be conjugated to one or more biotin binding sites of the avidin protein. In certain embodiments, the binding group B of the linker may comprise a click chemistry handle, and the binding moiety of the surface may comprise a complementary click chemistry handle capable of undergoing a click chemistry reaction with the click chemistry handle of the binding group B of the linker. For example, when the click chemistry handle of the binding group B of the linker comprises an azide, nitrile oxide, or tetrazine, then the click chemistry handle of the binding moiety of the surface may comprise an alkyne or strained alkene. Conversely, when the click chemistry handle of the binding group B of the linker comprises an alkyne or strained alkene, then the click chemistry handle of the binding moiety of the surface may comprise an azide, nitrile oxide, or tetrazine.

In some embodiments, the sample preparation method comprises removing (e.g., washing away) any peptide fragments that are not functionalized (e.g., do not comprise a click chemistry handle). In certain embodiments, the sample preparation method comprises removing (e.g., washing away) any peptide fragments that are not immobilized to a surface (e.g., a surface of a sample well).

Some aspects are directed to a kit comprising one or more reagents (e.g., one or more sample preparation reagents). In some embodiments, the kit comprises a protected thiol compound. In certain embodiments, the protected thiol compound is a compound of Formula (I), or a salt thereof, as described herein. In certain embodiments, the protected thiol compound comprises a click chemistry handle (e.g., an azide, an alkyne, an alkene, a nitrile oxide, a tetrazine). In some embodiments, the kit comprises one or more bases. In certain embodiments, the one or more bases comprise barium hydroxide and/or sodium hydroxide. In some embodiments, the kit comprises a linker. In certain embodiments, the linker is a compound of Formula (III), as described herein. In some embodiments, the kit comprises a polypeptide-cleaving agent. In certain embodiments, the polypeptide-cleaving agent comprises a protease. Non-limiting examples of suitable proteases include Arg-C (also referred to as clostripain), Lys-C, Glu-C, trypsin, chymotrypsin, Asp-N, Lys-N, Asp-X, thrombin, elastase, subtilisin, pepsin, thermolysin, proteinase K, carboxypeptidase A, carboxypeptidase B, cathepsin C, caspase, glutamyl endopeptidase, and proline endopeptidase. In some embodiments, the polypeptide-cleaving agent comprises a non-enzymatic reagent. Non-limiting examples of suitable non-enzymatic reagents include cyanogen bromide, formic acid, hydrochloric acid, hydroxylamine, 2-nitro-5-thiocyanatobenzoic acid, BNPS-skatole, and iodosobenzoic acid. In some embodiments, the kit comprises a quenching agent. In certain embodiments, the quenching agent is capable of quenching a polypeptide-cleaving reaction (e.g., enzymatic digestion). Non-limiting examples of quenching agents include acetic acid, trifluoroacetic acid, and hydrochloric acid.

In some embodiments, a sample preparation kit comprises a protected thiol compound (e.g., a compound of Formula (I), or a salt thereof) and a linker (e.g., a compound of Formula (III)). In some embodiments, a sample preparation kit comprises a protected thiol compound, a linker, and a base. In some embodiments, a sample preparation kit comprises a protected thiol compound, a linker, and a polypeptide-cleaving agent. In some embodiments, a sample preparation kit comprises a protected thiol compound, a linker, and a quenching agent. In some embodiments, a sample preparation kit comprises a protected thiol compound, a linker, a base, and a polypeptide-cleaving agent. In some embodiments, a sample preparation kit comprises a protected thiol compound, a linker, a base, and a quenching agent. In some embodiments, a sample preparation kit comprises a protected thiol compound, a linker, a polypeptide-cleaving agent, and a quenching agent. In some embodiments, a sample preparation kit comprises a protected thiol compound, a linker, a base, a polypeptide-cleaving agent, and a quenching agent.

Some aspects are directed to a method of enriching a sample comprising at least one polypeptide comprising a target residue. In certain embodiments, the sample comprises a plurality of polypeptides. In some instances, one or more polypeptides of the plurality of peptides comprise a target residue and one or more polypeptides of the plurality of peptides do not comprise a target residue. In certain instances, a molar ratio of polypeptides that do not comprise a target residue to polypeptides that do comprise a target residue is at least 2:1, at least 3:1, at least 4:1, at least 5:1, at least 10:1, at least 15:1, at least 20:1, at least 50:1, or at least 100:1. In certain instances, a molar ratio of polypeptides that do not comprise a target residue to polypeptides that do comprise a target residue is in a range between 2:1 and 5:1, 2:1 and 10:1, 2:1 and 15:1, 2:1 and 20:1, 2:1 and 50:1, 2:1 and 100:1, 5:1 and 10:1, 5:1 and 15:1, 5:1 and 20:1, 5:1 and 50:1, 5:1 and 100:1, 10:1 and 20:1, 10:1 and 50:1, 10:1 and 100:1, 20:1 and 50:1, 20:1 and 100:1, and 50:1 and 100:1.

In some embodiments, the target residue comprises a post-translational modification. Non-limiting examples of post-translational modifications include acetylation, ADP-ribosylation, caspase cleavage, citrullination, formylation, N-linked glycosylation, O-linked glycosylation, hydroxylation, methylation, myristoylation, neddylation, nitration, oxidation, palmitoylation, phosphorylation, prenylation, S-nitrosylation, sulfation, sumoylation, and ubiquitination. In certain embodiments, the target residue is a phosphoserine residue.

In some embodiments, the method of enriching a sample comprising at least one polypeptide comprising a target residue comprises selectively conjugating the target residue to a linker. In certain embodiments, the linker comprises a first binding moiety. In certain cases, the first binding moiety comprises at least one biotin moiety (e.g., a bis-biotin moiety), an avidin protein (e.g., a streptavidin), and/or a click chemistry handle (e.g., an azide, tetrazine, nitrile oxide, alkyne, or alkene). In some instances, the linker is a compound of Formula (III), as described herein. In certain instances, the target residue comprises phosphoserine, and selectively conjugating the target residue to a linker comprises converting a phosphoserine residue of a polypeptide or peptide fragment to a dehydroalanine residue, reacting the dehydroalanine residue with a protected thiol compound (e.g., a compound of Formula (I), or a salt thereof) to provide a functionalized dehydroalanine residue, and conjugating the functionalized dehydroalanine residue to a linker (e.g., a compound of Formula (III)).

In some embodiments, the method of enriching a sample comprising at least one polypeptide comprising a target residue comprises immobilizing the linker to a surface (e.g., a surface of a sample well). In certain embodiments, the surface comprises a second binding moiety. In some cases, the second binding moiety of the surface is configured to covalently or non-covalently bind, directly or indirectly, to the first binding moiety of the linker. In certain cases, the first binding moiety comprises at least one biotin moiety (e.g., a bis-biotin moiety) and/or an avidin protein (e.g., a streptavidin), and the second binding moiety comprises at least one biotin moiety (e.g., a bis-biotin moiety) and/or an avidin protein (e.g., a streptavidin). In certain cases, the first binding moiety comprises a click chemistry handle, and the second binding moiety comprises a complementary click chemistry handle capable of undergoing a click chemistry reaction with the click chemistry handle of the first binding moiety. For example, when the click chemistry handle of the first binding moiety comprises an azide, nitrile oxide, or tetrazine, then the click chemistry handle of the second binding moiety may comprise an alkyne or strained alkene. Conversely, when the click chemistry handle of the first binding moiety comprises an alkyne or strained alkene, then the click chemistry handle of the second binding moiety may comprise an azide, nitrile oxide, or tetrazine. In some embodiments, the second binding moiety an azide, tetrazine, nitrile oxide, alkyne, or alkene.

In some embodiments, the method of enriching a sample comprising at least one polypeptide comprising a target residue comprises removing (e.g., washing away) any non-immobilized polypeptides or peptide fragments. In some embodiments, the remaining immobilized peptide fragments may be used in systems and methods for characterizing a polypeptide comprising a target residue, as described herein.

### Systems and Methods for Characterizing a Polypeptide Comprising a Target Residue

In some aspects, systems and methods of the disclosure may be utilized to characterize a polypeptide (e.g., a polypeptide comprising a target residue). In some embodiments, one or more characteristics of a polypeptide may be determined by evaluating single-molecule binding interactions between amino acid recognition molecules and the polypeptide while amino acids are progressively cleaved from a terminal end of the polypeptide (e.g., an N-terminal end of the polypeptide).

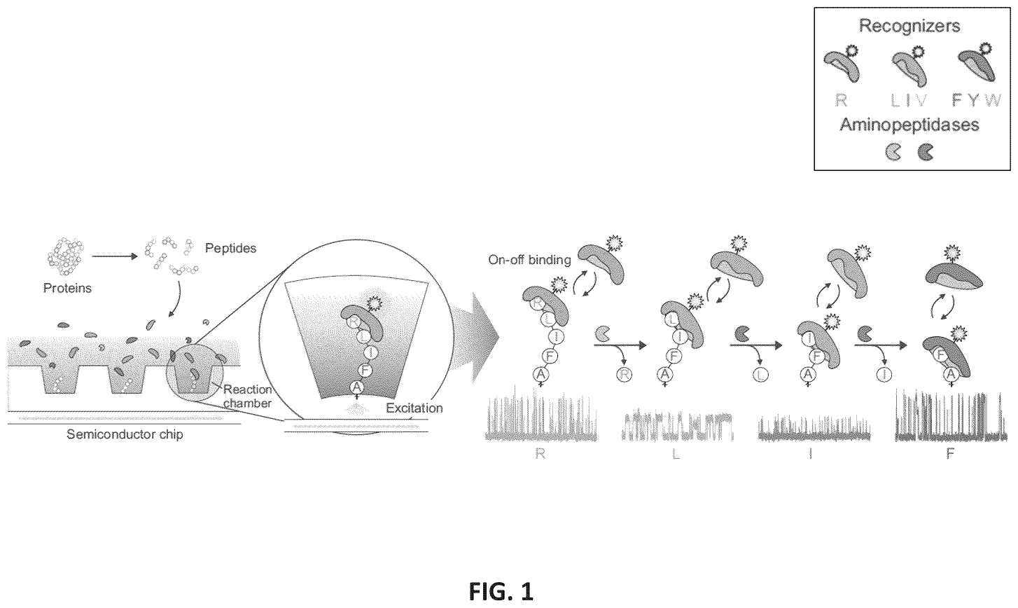

FIG. 1 shows a schematic illustration of an exemplary dynamic peptide sequencing reaction in which individual on-off binding events give rise to signal pulses of a signal output. As shown at left, a polypeptide sample may be fragmented into peptides, which are immobilized in sample wells of an array, where the immobilized peptides are exposed to one or more amino acid recognition molecules (also referred to as recognizers) and one or more cleaving reagents (e.g., aminopeptidases). As shown at right, an amino acid recognition molecule reversibly binds a terminal end of the peptide, and a detectable signal is produced while the recognition molecule is bound to the peptide. As the on-off binding of recognition molecules generally occurs at a faster rate than amino acid cleavage, the binding events preceding amino acid cleavage give rise to a series of signal pulses that can be used to determine at least one chemical characteristic of the peptide (and/or an originating polypeptide). In certain embodiments, determining at least one chemical characteristic of the peptide comprises detecting the presence or absence of a target residue. In certain embodiments, determining at least one chemical characteristic of the peptide comprises determining the location of a target residue in the peptide (and/or an originating polypeptide). In certain embodiments, determining at least one chemical characteristic of the peptide comprises determining if one or more amino acids comprise a post-translational modification. In certain embodiments, determining at least one chemical characteristic of the peptide comprises determining an identity of one or more amino acids of the peptide.

In some embodiments, the peptide is immobilized to a surface (e.g., a surface of a sample well) through a terminal end distal to the terminus to which one or more amino acid recognition molecules associate. In certain embodiments, for example, a first terminus (e.g., a C or N terminus) of a peptide is immobilized to a surface and the other terminus (an N or C terminus) is characterized (e.g., sequenced) as described herein. In certain embodiments, a peptide is characterized (e.g., sequenced) from its amino (N) terminus to its carboxy (C) terminus. In certain embodiments, a peptide is characterized (e.g., sequenced) from its carboxy (C) terminus to its amino (N) terminus.

In some embodiments, the peptide is immobilized to a surface (e.g., a surface of a sample well) at an amino acid other than a terminal amino acid. In certain embodiments, the surface comprises a binding moiety that is covalently or non-covalently bound, directly or indirectly, to an amino acid of the peptide other than a terminal amino acid. In some embodiments, the peptide is immobilized to the surface at a target residue. In certain embodiments, the surface comprises a binding moiety that is covalently or non-covalently bound, directly or indirectly, to a target residue of the peptide.

In some embodiments, the target residue comprises a post-translational modification. Non-limiting examples of post-translational modifications include acetylation, ADP-ribosylation, caspase cleavage, citrullination, formylation, N-linked glycosylation, O-linked glycosylation, hydroxylation, methylation, myristoylation, neddylation, nitration, oxidation, palmitoylation, phosphorylation, prenylation, S-nitrosylation, sulfation, sumoylation, and ubiquitination. In certain embodiments, the target residue is a phosphoserine residue. In certain embodiments, the target residue is a phosphoserine residue that has undergone one or more chemical reactions (e.g., conversion to a dehydroalanine residue, functionalization with a click chemistry handle).

In some embodiments, a system comprises an integrated device comprising a sample well. In some embodiments, the system further comprises a peptide that is immobilized to a surface of the sample well at a target residue. In certain embodiments, the target residue is not a terminal amino acid of the peptide (e.g., a C-terminal amino acid, an N-terminal amino acid). That is, the peptide may comprise one or more amino acids between a C-terminal amino acid and the target residue and one or more amino acids between an N-terminal amino acid and the target residue. In some embodiments, the integrated device further comprises one or more photodetectors, as described herein. In some embodiments, the system further comprises one or more amino acid recognition molecules. In certain instances, the one or more amino acid recognition molecules are labeled with one or more detectable labels. In some embodiments, the system further comprises one or more cleaving reagents. In certain instances, the one or more cleaving agents comprise one or more aminopeptidases.

In some embodiments, a method of characterizing a peptide comprises immobilizing the peptide to a surface of a sample well at a target residue. In certain embodiments, the target residue is not a terminal amino acid of the peptide. In some embodiments, the method further comprises contacting the peptide with one or more amino acid recognition molecules labeled with one or more detectable labels. In some embodiments, the method further comprises detecting one or more series of signal pulses indicative of binding events between the one or more amino acid recognition molecules and the peptide. In some embodiments, the method further comprises determining at least one chemical characteristic of the peptide (and/or an originating polypeptide). In certain embodiments, determining at least one chemical characteristic of the peptide comprises detecting the presence or absence of a target residue. In certain embodiments, determining at least one chemical characteristic of the peptide comprises determining the location of a target residue in the peptide (and/or an originating polypeptide). In certain embodiments, determining at least one chemical characteristic of the peptide comprises determining if one or more amino acids comprise a post-translational modification. In certain embodiments, determining at least one chemical characteristic of the peptide comprises determining an identity of one or more amino acids of the peptide.

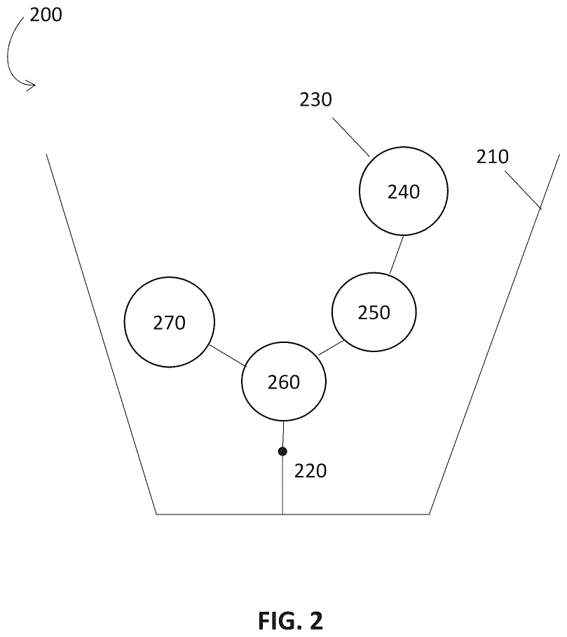

FIG. 2 shows a schematic illustration of an exemplary system in which a peptide is immobilized to a surface of a sample well at a target residue. In FIG. 2, system 200 comprises sample well 210. A surface (e.g., a bottom surface) of sample well 210 is functionalized with peptide-binding moiety 220. In some embodiments, peptide-binding moiety 220 is bound to peptide fragment 230 comprising N-terminal amino acid 240, amino acid 250, target residue 260, and C-terminal amino acid 270. As shown in FIG. 2, peptide-binding moiety 220 may be bound to peptide fragment 230 through target residue 260, which is not a terminal amino acid (e.g., a C-terminal amino acid, an N-terminal amino acid) of peptide fragment 230. In certain embodiments, peptide-bonding moiety 220 may be bound to target residue 260 via a linker, as described herein. In certain embodiments, target residue 260 comprises a phosphoserine residue. In certain embodiments, target residue 260 comprises a phosphoserine residue that has been reacted with one or more reagents (e.g., one or more bases, one or more protected thiol compounds) to form a functionalized phosphoserine derivative residue (e.g., a residue of Formula (II)).

In operation, a polypeptide may be digested (e.g., by one or more polypeptide-cleaving agents) to produce two or more peptide fragments, including one or more peptide fragments (e.g., peptide fragment 230) comprising a target residue (e.g., target residue 260). In some embodiments, peptide fragment 230 may be immobilized to a surface of sample well 210 through target residue 260, which is not a terminal amino acid of peptide fragment 230. In some embodiments, one or more labeled amino acid recognition molecules may reversibly bind to N-terminal amino acid 240 of peptide fragment 230, and a detectable signal may be produced while the recognition molecule is bound to N-terminal amino acid 240. A series of pulses may be obtained, which can be used to determine one or more characteristics of the peptide fragment. In some embodiments, a cleaving agent may cleave N-terminal amino acid 240 from peptide fragment 230, thereby exposing amino acid 250 as the next N-terminal amino acid of peptide fragment 230. This process may occur from one terminal end of the peptide fragment (e.g., from the N-terminal end of the peptide fragment) until target residue 260 is exposed at the N-terminal end of peptide fragment 230. Once target residue 260 is exposed, the peptide characterizing reaction may terminate. In some cases, the location at which the reaction terminates may provide information regarding the position of the target residue in the peptide fragment and, therefore, the originating polypeptide.

Some aspects are directed to a method of characterizing a phosphoserine residue of a polypeptide. In some embodiments, the method comprises converting the phosphoserine residue of the polypeptide to a dehydroalanine residue to provide a dehydroalanine-containing polypeptide. In some embodiments, the method comprises reacting the dehydroalanine-containing polypeptide with a thiol compound comprising a click chemistry handle (e.g., a compound of Formula (I), or a salt thereof) to provide a functionalized polypeptide comprising a functionalized dehydroalanine residue (e.g., a residue of Formula (II)). In some embodiments, the method comprises digesting the functionalized polypeptide to form two or more peptide fragments, wherein at least one peptide fragment is a functionalized peptide fragment comprising a functionalized dehydroalanine residue (e.g., a residue of Formula (II)). In certain embodiments, the method comprises conjugating the functionalized peptide fragment to a linker (e.g., a compound of Formula (III)). In certain embodiments, the method comprises immobilizing the linker to a surface of a sample well. In certain embodiments, the method comprises removing any peptide fragments that do not comprise a functionalized dehydroalanine residue (e.g., any non-immobilized peptide fragments).

In some embodiments, the method of characterizing a phosphoserine residue of a polypeptide comprises digesting the polypeptide to form two or more peptide fragments, wherein at least one peptide fragment comprises the phosphoserine residue. In some embodiments, the method comprises converting the phosphoserine residue of the at least one peptide fragment to a dehydroalanine residue of the at least one peptide fragment to provide a dehydroalanine-containing peptide fragment. In some embodiments, the method comprises reacting the dehydroalanine-containing peptide fragment with a thiol compound comprising a click chemistry handle (e.g., a compound of Formula (I), or a salt thereof) to provide a functionalized peptide fragment comprising a functionalized dehydroalanine residue (e.g., a residue of Formula (II)). In certain embodiments, the method comprises conjugating the functionalized peptide fragment to a linker (e.g., a compound of Formula (III)). In certain embodiments, the method comprises immobilizing the linker to a surface of a sample well. In certain embodiments, the method comprises removing any peptide fragments that do not comprise a functionalized dehydroalanine residue (e.g., any non-immobilized peptide fragments).

In some embodiments, the method comprises contacting the functionalized peptide fragment with one or more amino acid recognition molecules. In certain embodiments, the one or more amino acid recognition molecules comprise one or more detectable labels. In some embodiments, the method comprises detecting a first series of signal pulses indicative of binding events between the one or more amino acid recognition molecules and a first amino acid at a terminus of the functionalized peptide fragment. In some embodiments, the method comprises determining at least one chemical characteristic of an amino acid of the functionalized peptide fragment based on at least one characteristic of the first series of signal pulses. In certain embodiments, determining at least one chemical characteristic of an amino acid of the functionalized peptide fragment comprises identifying the amino acid, determining if the amino acid comprises a post-translational modification, detecting the presence of a phosphoserine residue, and/or determining the location of a phosphoserine residue. In certain embodiments, the at least one characteristic of the first series of signal pulses comprises pulse duration, interpulse duration, and/or cleavage rate. In some embodiments, the method comprises removing the first amino acid from the terminus of the functionalized peptide fragment. In certain embodiments, the first amino acid is removed by a cleaving agent. In some instances, the cleaving agent comprises an aminopeptidase. In some embodiments, the method comprises repeating the contacting, detecting, determining, and removing steps until the functionalized dehydroalanine residue is exposed at the terminus of the functionalized peptide fragment.

As described herein, one or more characteristics of the series of signal pulses may be determined, including signal pulse intensity, fluorescence lifetime, wavelength, signal pulse duration, interpulse duration, and/or cleavage rate, among others.

In some embodiments, the characteristic of the series of signal pulses may comprise intensity (e.g., average intensity of the series of signal pulses). Intensity may be determined based on an amount of charge carriers detected in the photodetection region which receives the emission light from the fluorescent labels.

In some embodiments, the characteristic of the series of signal pulses may comprise pulse wavelength (e.g., average pulse wavelength of the series of signal pulses). In particular, emission light from a particular fluorescent label may have a characteristic wavelength such that analyzing wavelength information of emission light may facilitate identification of one or more chemical characteristics of the sample. Wavelength of the emission light may be determined in any suitable manner, for example, using one or more optical filters and/or photodetection regions disposed at different depths.

In some embodiments, the characteristic of the series of signal pulses may comprise fluorescence lifetime (e.g., average fluorescence lifetime of the series of signal pulses). In particular, fluorescent labels, when excited by incident excitation light, fluoresce with a characteristic lifetime (e.g., a characteristic emission decay time period), such that analyzing the lifetime information of emission light may facilitate identification of one or more chemical characteristics of the sample to which the fluorescent dye is attached. Fluorescence lifetime, also referred to herein as simply “lifetime”, is a measure of the time which a fluorescent dye spends in the excited state before returning to a ground state and emitting a photon. In some embodiments, fluorescence lifetime information and/or other timing characteristics described herein may be obtained through techniques for time binning charge carriers generated by photons incident on a photodetection region (e.g., a photodiode).

In some embodiments, the characteristic of the series of signal pulses may comprise pulse duration (e.g., average pulse duration), also referred to herein as pulse width. Pulse duration refers to the interval of time measured across a pulse, in some embodiments, at the full width half maximum of a pulse. As described herein, dye-labeled amino acid recognizers periodically bind and unbind to the polypeptide (e.g., to the amino acid). When bound, the dye-labeled amino acid recognizers may become excited and emit emission light. The average duration of respective signal pulses emitted by the dye-labeled amino acid recognizers comprise the pulse duration of the fluorescent label.

In some embodiments, the characteristic of the series of signal pulses may comprise interpulse duration (e.g., average interpulse duration). Interpulse duration, also referred to herein as interpulse width, refers to the interval of time between adjacent pulses. As described herein, dye-labeled amino acid recognizers periodically bind and unbind to the polypeptide (e.g., to the amino acid). When bound, the dye-labeled amino acid recognizers may become excited and emit emission light. The average durations between signal pulses emitted by the fluorescent label comprise the interpulse duration of the fluorescent label.

In some embodiments, the characteristic of the series of signal pulses may comprise a cleavage rate/time (e.g., an average cleavage rate/time). For example, a terminal amino acid of the polypeptide may be cleaved from the polypeptide fragment disposed in the reaction chamber. In some embodiments, cleaving the terminal amino acid is performed by introducing a solution comprising aminopeptidases into the chamber. In some embodiments, the aminopeptidases may be included in the same solution as the sample chain of amino acids and/or amino acid recognizers. A cleavage rate or cleavage time may comprise a duration between cleavage events. Cleavage events may be determined based on distinguishing respective series of signal pulses between each other. For example, a first series of signal pulses may be indicative of a series of binding events between a first set of one or more amino acid recognizers and an amino acid, such as the terminal amino acid. A second series of signal pulses may be indicative of a series of binding events between a second set of one or more amino acid recognizers and a subsequent amino acid (e.g., an amino acid which becomes the terminal amino acid after the initial terminal amino acid is cleaved). The respective series of signal pulses may have different characteristics, as described herein, which may allow the respective series of signal pulses to be distinguished from each other. Each series of signal pulses may be referred to herein as a recognition segment. Each recognition segment therefore comprises a plurality of on-off binding events between a set of one or more amino acid recognizers and a respective amino acid. The cleavage time may comprise a duration of each recognition segment. In some embodiments, the at least one characteristic comprises a duration of time between recognition segments (e.g., an average intersegment duration).

As described herein, a method may comprise determining at least one chemical characteristic of a polypeptide. In some embodiments, determining at least one chemical characteristic comprises determining the type of amino acid that is present in the polypeptide, including at a terminal end of the polypeptide, and/or the types of amino acids that are present at one or more other positions in the polypeptide, such as downstream, proximate, or contiguous to the amino acid. In some embodiments, determining the type of amino acid comprises determining the amino acid identity, for example by determining which of the naturally-occurring 20 amino acids is present. In some embodiments, the type of amino acid is selected from alanine, arginine, asparagine, aspartic acid, cysteine, glutamine, glutamic acid, glycine, histidine, isoleucine, leucine, lysine, methionine, phenylalanine, proline, selenocysteine, serine, threonine, tryptophan, tyrosine, and valine.

As used herein, in some embodiments, “identifying,” “determining the identity,” “determining the type,” and like terms, in reference to an amino acid, include determination of an express identity of an amino acid as well as determination of a probability of an express identity of an amino acid. For example, in some embodiments, an amino acid is identified by determining a probability (e.g., from 0% to 100%) that the amino acid is of a specific type, or by determining a probability for each of a plurality of specific types. Accordingly, in some embodiments, the terms “amino acid sequence,” “polypeptide sequence,” and “protein sequence” as used herein may refer to the polypeptide or protein material itself and is not restricted to the specific sequence information (e.g., the succession of letters representing the order of amino acids from one terminus to another terminus) that biochemically characterizes a specific polypeptide or protein.

In some embodiments, determining at least one chemical characteristic of a polypeptide comprises determining a subset of potential amino acids that can be present in the polypeptide. In some embodiments, this can be accomplished by determining that an amino acid is not one or more specific amino acids (and therefore could be any of the other amino acids). In some embodiments, this can be accomplished by determining which of a specified subset of amino acids (e.g., based on size, charge, hydrophobicity, post-translational modification, binding properties) could be in the polypeptide (e.g., using a recognizer that binds to a specified subset of two or more amino acids).

In some embodiments, determining at least one chemical characteristic of a polypeptide comprises determining that an amino acid-whether the terminal amino acid or one or more of the amino acids downstream of the terminal amino acid-comprises a post-translational modification. As described herein, the post-translational modification may be to the terminal amino acid or may additionally or alternatively be to one or more other amino acids of the polypeptide. The post-translational modification may affect the series of signals emitted by a dye-labeled amino acid recognizer bound to the peptide (e.g., to the terminal amino acid and, in some embodiments, to one or more amino acids downstream of the terminal amino acid). In some embodiments, the series of signals emitted by the dye-labeled amino acid may be impacted by the post-translational modification even if the post-translational modification is to an amino acid which does not bind to the dye-labeled amino acid recognizer. Non-limiting examples of post-translational modifications include acetylation (e.g., acetylated lysine), ADP-ribosylation, caspase cleavage, citrullination, formylation, N-linked glycosylation (e.g., glycosylated asparagine), O-linked glycosylation (e.g., glycosylated serine, glycosylated threonine), hydroxylation, methylation (e.g., methylated lysine, methylated arginine), myristoylation (e.g., myristoylated glycine), neddylation, nitration (e.g., nitrated tyrosine), chlorination (e.g., chlorinated tyrosine), oxidation/reduction (e.g., oxidized cysteine, oxidized methionine), carbonylation (e.g., carbonylated lysine, carbonylated proline, carbonylated arginine, carbonylated threonine), palmitoylation (e.g., palmitoylated cysteine), phosphorylation, prenylation (e.g., prenylated cysteine), S-nitrosylation (e.g., S-nitrosylated cysteine, S-nitrosylated methionine), sulfation (e.g., sulfated tyrosine), glycation (e.g., glycated lysine), sumoylation (e.g., sumoylated lysine), and ubiquitination (e.g., ubiquitinated lysine).

In some embodiments, determining at least one chemical characteristic of a polypeptide comprises determining that an amino acid comprises a phosphorylated side chain. For example, in some embodiments, determining at least one chemical characteristic of a polypeptide comprises determining that an amino acid comprises phosphorylated threonine (e.g., phospho-threonine). In some embodiments, determining at least one chemical characteristic of a polypeptide comprises determining that an amino acid comprises phosphorylated tyrosine (e.g., phospho-tyrosine). In some embodiments, determining at least one chemical characteristic of a polypeptide comprises determining that an amino acid comprises phosphorylated serine (e.g., phospho-serine). In some embodiments, determining at least one chemical characteristic of a polypeptide comprises determining a location of a phosphorylated serine in the polypeptide.

In some embodiments, determining at least one chemical characteristic of a polypeptide comprises determining that an amino acid comprises a chemically modified variant, an unnatural amino acid, or a proteinogenic amino acid such as selenocysteine and pyrrolysine. Examples of unnatural amino acids include, without limitation, 2-naphthyl-alanine, statine, homoalanine, α-amino acid, 132-amino acid, 133-amino acid, γ-amino acid, 3-pyridyl-alanine, 4-fluorophenyl-alanine, cyclohexyl-alanine, N-alkyl amino acid, peptoid amino acid, homo-cysteine, penicillamine, 3-nitro-tyrosine, homo-phenyl-alanine, t-leucine, hydroxy-proline, 3-Abz, 5-F-tryptophan, and azabicyclo-[2.2.1]heptane.

In some embodiments, determining at least one chemical characteristic of a polypeptide comprises determining that an amino acid comprises an oxidative modification. For example, as described herein, amino acid recognizers of the disclosure are capable of distinguishing between oxidized methionine and its unmodified variant. In some embodiments, the oxidative modification comprises an oxidatively-damaged side chain of an amino acid. In some embodiments, the oxidative modification comprises an oxidatively-damaged side chain of an amino acid. In some embodiments, the oxidatively-damaged side chain comprises a cysteine-derived product (e.g., disulfide, sulfinic acid, sulfonic acid, sulfenic acid, S-nitrosocysteine), a tyrosine-derived product (e.g., di-tyrosine, 3,4-dihydroxyphenylalanine, 3-chlorotyrosine, 3-nitrotyrosine), a histidine-derived product (e.g., 2-oxohistidine, 4-hydroxy-2-oxohistidine, di-histidine, asparagine, aspartic acid, urea), a methionine-derived product (e.g., sulfoxide, sulfone), a tryptophan-derived product (e.g., di-tryptophan, N-formylkynurenine, kynurenine, 2-oxo-tryptophan oxindolylalanine, 6-nitrotryptophan, hydroxytryptophan), a phenylalanine-derived product (e.g., meta-tyrosine, ortho-tyrosine), or a generic side-chain product (e.g., alcohol, hydroperoxide, aldehyde/ketone carbonyl). Examples of oxidatively damaged amino acids are known in the art, see, e.g., Hawkins, C. L., Davies, M. J. Detection, identification, and quantification of oxidative protein modifications. J Biol Chem. 2019 Dec. 20; 294(51):19683-19708.

In some embodiments, determining at least one chemical characteristic of a polypeptide comprises determining that an amino acid comprises a side chain characterized by one or more biochemical properties. For example, an amino acid may comprise a nonpolar aliphatic side chain, a positively charged side chain, a negatively charged side chain, a nonpolar aromatic side chain, or a polar uncharged side chain. Non-limiting examples of an amino acid comprising a nonpolar aliphatic side chain include alanine, glycine, valine, leucine, methionine, and isoleucine. Non-limiting examples of an amino acid comprising a positively charged side chain includes lysine, arginine, and histidine. Non-limiting examples of an amino acid comprising a negatively charged side chain include aspartate and glutamate. Non-limiting examples of an amino acid comprising a nonpolar, aromatic side chain include phenylalanine, tyrosine, and tryptophan. Non-limiting examples of an amino acid comprising a polar uncharged side chain include serine, threonine, cysteine, proline, asparagine, and glutamine. As described herein, the at least one characteristic of the signal pulses may be used to determine at least one chemical characteristic of at least two amino acids. Accordingly, signal pulses from a dye-labeled first type of amino acid recognizer that binds to an amino acid, such as the terminal amino acid or an internal amino acid, may be used to determine one or more chemical characteristics of multiple amino acids. The inventors have recognized that such techniques are advantageous. For example, such techniques may allow for determining chemical characteristics of amino acids which are unrecognized. Such amino acids may be unrecognizable by any amino acid recognizers present in a reaction chamber, in some instances. Such techniques may also save time and/or require less signal collection. Accordingly, obtaining information regarding multiple amino acids based on fewer series of signal pulses and/or using fewer recognizers is advantageous.

In some embodiments, determining at least one chemical characteristic of a polypeptide comprises determining that at least one amino acid is bound (e.g., via a covalent or non-covalent interaction) to a binding component. Non-limiting examples of suitable binding components include a nucleic acid (e.g., DNA, RNA), a linker, and an antibody. In some instances, one or more amino acids of a polypeptide may be bound to a nucleic acid via one or more non-covalent interactions. In some instances, one or more amino acids of a polypeptide may be bound to a linker via one or more covalent interactions.

In some embodiments, one or more characteristics of a first series of pulse signals indicative of a first series of binding events between one or more amino acid recognizers and a first amino acid of a polypeptide (e.g., a terminal amino acid, an internal amino acid) may be impacted by one or more chemical characteristics of the polypeptide. In certain instances, one or more modifications of one or more amino acids (e.g., post-translational modifications, presence of binding components) may promote a covalent or non-covalent interaction between one or more amino acid recognizers and the first amino acid (e.g., through electrostatic attraction, pi stacking, hydrogen bond formation, etc.), thereby increasing pulse duration. In certain instances, one or more modifications of one or more amino acids (e.g., post-translational modifications, presence of binding components) may discourage a covalent or non-covalent interaction between one or more amino acid recognizers and the first amino acid (e.g., through electrostatic repulsion, steric hindrance, etc.), thereby decreasing pulse duration.

Compositions and methods for characterizing a polypeptide and analyzing data obtained therefrom are described more fully in PCT International Publication No. WO2020102741A1, filed Nov. 15, 2019, and PCT International Publication No. WO2021236983A2, filed May 20, 2021, each of which is incorporated by reference in its entirety. Examples of luminescent labels, linkers, and other reagents for use in accordance with the disclosure are described in U.S. applications entitled “Polypeptidyl Linkers” and “Luminescently Labeled Oligonucleotide Structures and Associated Systems and Methods,” filed on even date herewith, the relevant contents of each of which are incorporated herein by reference in their entireties.

### Amino Acid Recognition Molecules

In some embodiments, methods provided herein comprise contacting a polypeptide with an amino acid recognition molecule, which may or may not comprise a label, that selectively binds at least one type of terminal amino acid. As used herein, in some embodiments, a terminal amino acid may refer to an amino-terminal amino acid of a polypeptide or a carboxy-terminal amino acid of a polypeptide. In some embodiments, a labeled recognition molecule selectively binds one type of terminal amino acid over other types of terminal amino acids. In some embodiments, a labeled recognition molecule selectively binds one type of terminal amino acid over an internal amino acid of the same type. In yet other embodiments, a labeled recognition molecule selectively binds one type of amino acid at any position of a polypeptide, e.g., the same type of amino acid as a terminal amino acid and an internal amino acid.

As used herein, in some embodiments, the term “bond” or “bonds” refers to any non-covalent interaction (e.g., a hydrogen bond, a van der Waals interaction, an aromatic interaction, an electrostatic interaction) or covalent interaction between specified binding components or any plurality thereof, and the terms “bind,” “binding,” “bound,” and like terms refer to the formation and/or existence of any such bonds. As an illustrative example, a binding event between an amino acid recognizer and an amino acid may comprise the formation of one or more non-covalent or covalent interactions between the amino acid recognizer and the amino acid.

As used herein, in some embodiments, a type of amino acid refers to one of the twenty naturally occurring amino acids or a subset of types thereof. In some embodiments, a type of amino acid refers to a modified variant of one of the twenty naturally occurring amino acids or a subset of unmodified and/or modified variants thereof. Examples of modified amino acid variants include, without limitation, post-translationally-modified variants (e.g., acetylation, ADP-ribosylation, caspase cleavage, citrullination, formylation, N-linked glycosylation, O-linked glycosylation, hydroxylation, methylation, myristoylation, neddylation, nitration, oxidation, palmitoylation, phosphorylation, prenylation, S-nitrosylation, sulfation, sumoylation, and ubiquitination), chemically modified variants, unnatural amino acids, and proteinogenic amino acids such as selenocysteine and pyrrolysine. In some embodiments, a subset of types of amino acids includes more than one and fewer than twenty amino acids having one or more similar biochemical properties. For example, in some embodiments, a type of amino acid refers to one type selected from amino acids with charged side chains (e.g., positively and/or negatively charged side chains), amino acids with polar side chains (e.g., polar uncharged side chains), amino acids with nonpolar side chains (e.g., nonpolar aliphatic and/or aromatic side chains), and amino acids with hydrophobic side chains.

In some embodiments, methods provided herein comprise contacting a polypeptide with one or more labeled recognition molecules that selectively bind one or more types of terminal amino acids. As an illustrative and non-limiting example, where four labeled recognition molecules are used in a method of the disclosure, any one recognition molecule selectively binds one type of terminal amino acid that is different from another type of amino acid to which any of the other three selectively binds (e.g., a first recognition molecule binds a first type, a second recognition molecule binds a second type, a third recognition molecule binds a third type, and a fourth recognition molecule binds a fourth type of terminal amino acid). For the purposes of this discussion, one or more labeled recognition molecules in the context of a method described herein may be alternatively referred to as a set of labeled recognition molecules.

In some embodiments, a set of labeled recognition molecules comprises at least one and up to six labeled recognition molecules. For example, in some embodiments, a set of labeled recognition molecules comprises one, two, three, four, five, or six labeled recognition molecules. In some embodiments, a set of labeled recognition molecules comprises ten or fewer labeled recognition molecules. In some embodiments, a set of labeled recognition molecules comprises eight or fewer labeled recognition molecules. In some embodiments, a set of labeled recognition molecules comprises six or fewer labeled recognition molecules. In some embodiments, a set of labeled recognition molecules comprises four or fewer labeled recognition molecules. In some embodiments, a set of labeled recognition molecules comprises three or fewer labeled recognition molecules. In some embodiments, a set of labeled recognition molecules comprises two or fewer labeled recognition molecules. In some embodiments, a set of labeled recognition molecules comprises four labeled recognition molecules. In some embodiments, a set of labeled recognition molecules comprises at least two and up to twenty (e.g., at least two and up to ten, at least two and up to eight, at least four and up to twenty, at least four and up to ten) labeled recognition molecules. In some embodiments, a set of labeled recognition molecules comprises more than twenty (e.g., 20 to 25, 20 to 30) recognition molecules. It should be appreciated, however, that any number of recognition molecules may be used in accordance with a method of the disclosure to accommodate a desired use.

In accordance with the disclosure, in some embodiments, one or more types of amino acids are identified by detecting luminescence of a labeled recognition molecule. In some embodiments, a labeled recognition molecule comprises a recognition molecule that selectively binds one type of amino acid and a luminescent label having a luminescence that is associated with the recognition molecule. In this way, the luminescence (e.g., luminescence lifetime, luminescence intensity, and other luminescence properties described elsewhere herein) may be associated with the selective binding of the recognition molecule to identify an amino acid of a polypeptide. In some embodiments, a plurality of types of labeled recognition molecules may be used in a method according to the disclosure, where each type comprises a luminescent label having a luminescence that is uniquely identifiable from among the plurality. In some embodiments, the luminescent label of each type of labeled recognition molecule is uniquely identifiable from among the plurality by luminescence intensity alone. Suitable luminescent labels may include luminescent molecules, such as fluorophore dyes, and are described elsewhere herein.

In some embodiments, an amino acid recognition molecule may be engineered by one skilled in the art using conventionally known techniques. In some embodiments, desirable properties may include an ability to bind selectively and with high affinity to one type of amino acid only when it is located at a terminus (e.g., an N-terminus or a C-terminus) of a polypeptide. In yet other embodiments, desirable properties may include an ability to bind selectively and with high affinity to one type of amino acid when it is located at a terminus (e.g., an N-terminus or a C-terminus) of a polypeptide and when it is located at an internal position of the polypeptide. In some embodiments, desirable properties include an ability to bind selectively and with low affinity (e.g., with a K_{D} of about 50 nM or higher, for example, between about 50 nM and about 50 μM, between about 100 nM and about 10 μM, between about 500 nM and about 50 μM) to more than one type of amino acid. For example, in some aspects, the disclosure provides methods of sequencing by detecting reversible binding interactions during a polypeptide degradation process. Advantageously, such methods may be performed using a recognition molecule that reversibly binds with low affinity to more than one type of amino acid (e.g., a subset of amino acid types).

As used herein, in some embodiments, the terms “selective” and “specific” (and variations thereof, e.g., selectively, specifically, selectivity, specificity) refer to a preferential binding interaction. For example, in some embodiments, an amino acid recognition molecule that selectively binds one type of amino acid preferentially binds the one type over another type of amino acid. A selective binding interaction will discriminate between one type of amino acid (e.g., one type of terminal amino acid) and other types of amino acids (e.g., other types of terminal amino acids), typically more than about 10- to 100-fold or more (e.g., more than about 1,000- or 10,000-fold). Accordingly, it should be appreciated that a selective binding interaction can refer to any binding interaction that is uniquely identifiable to one type of amino acid over other types of amino acids. For example, in some aspects, the disclosure provides methods of polypeptide sequencing by obtaining data indicative of association of one or more amino acid recognition molecules with a polypeptide molecule. In some embodiments, the data comprises a series of signal pulses corresponding to a series of reversible amino acid recognition molecule binding interactions with an amino acid of the polypeptide molecule, and the data may be used to determine the identity of the amino acid. As such, in some embodiments, a “selective” or “specific” binding interaction refers to a detected binding interaction that discriminates between one type of amino acid and other types of amino acids.

In some embodiments, an amino acid recognition molecule binds one type of amino acid with a dissociation constant (K_{D}) of less than about 10⁻⁶ M (e.g., less than about 10⁻⁷ M, less than about 10⁻⁸ M, less than about 10⁻⁹ M, less than about 10⁻¹⁰ M, less than about 10⁻¹¹ M, less than about 10⁻¹² M, to as low as 10⁻¹⁶ M) without significantly binding to other types of amino acids. In some embodiments, an amino acid recognition molecule binds one type of amino acid (e.g., one type of terminal amino acid) with a K_{D} of less than about 100 nM, less than about 50 nM, less than about 25 nM, less than about 10 nM, or less than about 1 nM. In some embodiments, an amino acid recognition molecule binds one type of amino acid with a K_{D} of between about 50 nM and about 50 μM (e.g., between about 50 nM and about 500 nM, between about 50 nM and about 5 μM, between about 500 nM and about 50 μM, between about 5 μM and about 50 μM, or between about 10 μM and about 50 μM). In some embodiments, an amino acid recognition molecule binds one type of amino acid with a K_{D} of about 50 nM.

In some embodiments, an amino acid recognition molecule binds two or more types of amino acids with a K_{D} of less than about 10⁻⁶ M (e.g., less than about 10⁻⁷ M, less than about 10⁻⁸ M, less than about 10⁻⁹ M, less than about 10⁻¹⁰ M, less than about 10⁻¹¹ M, less than about 10⁻¹² M, to as low as 10⁻¹⁶ M). In some embodiments, an amino acid recognition molecule binds two or more types of amino acids with a K_{D} of less than about 100 nM, less than about 50 nM, less than about 25 nM, less than about 10 nM, or less than about 1 nM. In some embodiments, an amino acid recognition molecule binds two or more types of amino acids with a K_{D} of between about 50 nM and about 50 μM (e.g., between about 50 nM and about 500 nM, between about 50 nM and about 5 μM, between about 500 nM and about 50 μM, between about 5 μM and about 50 μM, or between about 10 μM and about 50 μM). In some embodiments, an amino acid recognition molecule binds two or more types of amino acids with a K_{D} of about 50 nM.

In some embodiments, an amino acid recognition molecule binds at least one type of amino acid with a dissociation rate (k_{off}) of at least 0.1 s⁻¹. In some embodiments, the dissociation rate is between about 0.1 s⁻¹ and about 1,000 s⁻¹ (e.g., between about 0.5 s⁻¹ and about 500 s⁻¹, between about 0.1 s⁻¹ and about 100 s⁻¹, between about 1 s⁻¹ and about 100 s⁻¹, or between about 0.5 s⁻¹ and about 50 s⁻¹). In some embodiments, the dissociation rate is between about 0.5 s⁻¹ and about 20 s⁻¹. In some embodiments, the dissociation rate is between about 2 s⁻¹ and about 20 s⁻¹. In some embodiments, the dissociation rate is between about 0.5 s⁻¹ and about 2 s⁻¹.

In some embodiments, the value for K_{D} or k_{off} can be a known literature value, or the value can be determined empirically. In some embodiments, the value for k_{off} can be determined empirically based on signal pulse information obtained in a single-molecule assay as described elsewhere herein. For example, the value for k_{off} can be approximated by the reciprocal of the mean pulse duration. In some embodiments, an amino acid recognition molecule binds two or more types of amino acids with a different K_{D} or k_{off} for each of the two or more types. In some embodiments, a first K_{D} or k_{off} for a first type of amino acid differs from a second K_{D} or k_{off} for a second type of amino acid by at least 10% (e.g., at least 25%, at least 50%, at least 100%, or more). In some embodiments, the first and second values for K_{D} or k_{off} differ by about 10-25%, 25-50%, 50-75%, 75-100%, or more than 100%, for example by about 2-fold, 3-fold, 4-fold, 5-fold, or more.

As described herein, an amino acid recognition molecule may be any biomolecule capable of selectively or specifically binding one molecule over another molecule (e.g., one type of amino acid over another type of amino acid). In some embodiments, a recognition molecule is not a peptidase or does not have peptidase activity. For example, in some embodiments, methods of polypeptide sequencing of the disclosure involve contacting a polypeptide molecule with one or more recognition molecules and a cleaving reagent. In such embodiments, the one or more recognition molecules do not have peptidase activity, and removal of one or more amino acids from the polypeptide molecule (e.g., amino acid removal from a terminus of the polypeptide molecule) is performed by the cleaving reagent.

Recognition molecules include, for example, proteins and nucleic acids, which may be synthetic or recombinant. In some embodiments, a recognition molecule may be an antibody or an antigen-binding portion of an antibody, an SH2 domain-containing protein or fragment thereof, or an enzymatic biomolecule, such as a peptidase, an aminotransferase, a ribozyme, an aptazyme, or a tRNA synthetase, including aminoacyl-tRNA synthetases and related molecules described in U.S. patent application Ser. No. 15/255,433, filed Sep. 2, 2016, titled “MOLECULES AND METHODS FOR ITERATIVE POLYPEPTIDE ANALYSIS AND PROCESSING.”

In some embodiments, a recognition molecule of the disclosure is a degradation pathway protein. Examples of degradation pathway proteins suitable for use as recognition molecules include, without limitation, N-end rule pathway proteins, such as Arg/N-end rule pathway proteins, Ac/N-end rule pathway proteins, and Pro/N-end rule pathway proteins. In some embodiments, a recognition molecule is an N-end rule pathway protein selected from a Gid protein (e.g., Gid4 or Gid10 protein), a UBR-box protein (e.g., UBR1, UBR2) or UBR-box domain-containing protein fragment thereof, a p62 protein or ZZ domain-containing fragment thereof, and a ClpS protein (e.g., ClpS1, ClpS2). Accordingly, in some embodiments, a labeled recognition molecule comprises a degradation pathway protein. In some embodiments, a labeled recognition molecule comprises a ClpS protein.

In some embodiments, a recognition molecule of the disclosure is a ClpS protein, such as Agrobacterium tumifaciens ClpS1, Agrobacterium tumifaciens ClpS2, Synechococcus elongatus ClpS1, Synechococcus elongatus ClpS2, Thermosynechococcus elongatus ClpS, Escherichia coli ClpS, or Plasmodium falciparum: ClpS. In some embodiments, the recognition molecule is an L/F transferase, such as Escherichia coli leucyl/phenylalanyl-tRNA-protein transferase. In some embodiments, the recognition molecule is a D/E leucyltransferase, such as Vibrio vulnificus Aspartate/glutamate leucyltransferase Bpt. In some embodiments, the recognition molecule is a UBR protein or UBR-box domain, such as the UBR protein or UBR-box domain of human UBR1 and UBR2 or Saccharomyces cerevisiae UBR1. In some embodiments, the recognition molecule is a p62 protein, such as H. sapiens p62 protein or Rattus norvegicus p62 protein, or truncation variants thereof that minimally include a ZZ domain. In some embodiments, the recognition molecule is a Gid4 protein, such as H. sapiens GID4 or Saccharomyces cerevisiae GID4. In some embodiments, the recognition molecule is a Gid10 protein, such as Saccharomyces cerevisiae GID10. In some embodiments, the recognition molecule is an N-meristoyltransferase, such as Leishmania major N-meristoyltransferase or H. sapiens N-meristoyltransferase NMT1. In some embodiments, the recognition molecule is a BIR2 protein, such as Drosophila melanogaster BIR2. In some embodiments, the recognition molecule is a tyrosine kinase or SH2 domain of a tyrosine kinase, such as H. sapiens Fyn SH2 domain, H. sapiens Src tyrosine kinase SH2 domain, or variants thereof, such as H. sapiens Fyn SH2 domain triple mutant superbinder. In some embodiments, the recognition molecule is an antibody or antibody fragment, such as a single-chain antibody variable fragment (scFv) against phosphotyrosine or another post-translationally modified amino acid variant described herein.

In some embodiments, an amino acid recognition molecule comprises a single polypeptide having tandem copies of two or more amino acid binding proteins (e.g., two or more binders).

In some embodiments, a recognition molecule of the disclosure is an amino acid binding protein which can be used with other types of amino acid binding molecules, such as a peptidase and/or a nucleic acid aptamer, in a sequencing method. A peptidase, also referred to as a protease or proteinase, is an enzyme that catalyzes the hydrolysis of a peptide bond. Peptidases digest polypeptides into shorter fragments and may be generally classified into endopeptidases and exopeptidases, which cleave a polypeptide chain internally and terminally, respectively. In some embodiments, a labeled recognition molecule comprises a peptidase that has been modified to inactivate exopeptidase or endopeptidase activity. In this way, the labeled recognition molecule selectively binds without also cleaving the amino acid from a polypeptide. In yet other embodiments, a peptidase that has not been modified to inactivate exopeptidase or endopeptidase activity may be used with an amino acid binding protein of the disclosure. For example, in some embodiments, a labeled recognition molecule comprises a labeled exopeptidase.

In some embodiments, an amino acid recognition molecule comprises one or more labels. In some embodiments, the one or more labels comprise a luminescent label or a conductivity label as described elsewhere herein. In some embodiments, the one or more labels comprise one or more polyol moieties (e.g., one or more moieties selected from dextran, polyvinylpyrrolidone, polyethylene glycol, polypropylene glycol, polyoxyethylene glycol, and polyvinyl alcohol). For example, in some embodiments, an amino acid recognition molecule is PEGylated. In some embodiments, polyol modification (e.g., PEGylation) can limit the extent of non-specific sticking to a substrate (e.g., sequencing chip) surface. In some embodiments, polyol modification can limit the extent of aggregation or interaction between an amino acid recognition molecule with other recognition molecules, with a cleaving reagent, or with other species present in a sequencing reaction mixture. PEGylation can be performed by incubating a recognition molecule (e.g., an amino acid binding protein, such as a ClpS protein) with mPEG4-NHS ester, which labels primary amines such as surface-exposed lysine side chains. Other types of PEG and other methods of polyol modification are known in the art.

In some embodiments, the one or more labels comprise a tag sequence. For example, in some embodiments, an amino acid recognition molecule comprises a tag sequence that provides one or more functions other than amino acid binding. In some embodiments, a tag sequence comprises at least one biotin ligase recognition sequence that permits biotinylation of the recognition molecule (e.g., incorporation of one or more biotin molecules, including biotin and bis-biotin moieties). In some embodiments, the tag sequence comprises two biotin ligase recognition sequences oriented in tandem. In some embodiments, a biotin ligase recognition sequence refers to an amino acid sequence that is recognized by a biotin ligase, which catalyzes a covalent linkage between the sequence and a biotin molecule. Each biotin ligase recognition sequence of a tag sequence can be covalently linked to a biotin moiety, such that a tag sequence having multiple biotin ligase recognition sequences can be covalently linked to multiple biotin molecules. A region of a tag sequence having one or more biotin ligase recognition sequences can be generally referred to as a biotinylation tag or a biotinylation sequence. In some embodiments, a bis-biotin or bis-biotin moiety can refer to two biotins bound to two biotin ligase recognition sequences oriented in tandem. Additional examples of functional sequences in a tag sequence include purification tags, cleavage sites, and other moieties useful for purification and/or modification of recognition molecules.

Examples of amino acid recognition molecules (e.g., amino acid binding proteins) for use in accordance with the disclosure are described more fully in PCT International Application No. PCT/US2019/061831, filed Nov. 15, 2019, and PCT International Application No. PCT/US2021/033493, filed May 20, 2021, the relevant contents of which are incorporated by reference in their entireties.

### Cleaving Reagents

In some embodiments, one or more cleaving reagents comprise a peptidase. A “peptidase,” “protease,” or “proteinase” is an enzyme that catalyzes the hydrolysis of a peptide bond. Peptidases digest polypeptides into shorter fragments and may be generally classified into endopeptidases and exopeptidases, which cleave a polypeptide chain internally and terminally, respectively.

In certain embodiments, one or more cleaving agents comprise an exopeptidase. An exopeptidase generally requires a polypeptide substrate to comprise at least one of a free amino group at its amino-terminus or a free carboxyl group at its carboxy-terminus. In some embodiments, an exopeptidase in accordance with the disclosure hydrolyses a bond at or near a terminus of a polypeptide. In some embodiments, an exopeptidase hydrolyses a bond not more than three residues from a polypeptide terminus. For example, in some embodiments, a single hydrolysis reaction catalyzed by an exopeptidase cleaves a single amino acid, a dipeptide, or a tripeptide from a polypeptide terminal end.

In some embodiments, an exopeptidase in accordance with the disclosure is an aminopeptidase or a carboxypeptidase, which cleaves a single amino acid from an amino- or a carboxy-terminus, respectively. In some embodiments, an exopeptidase in accordance with the disclosure is a dipeptidyl-peptidase or a peptidyl-dipeptidase, which cleave a dipeptide from an amino- or a carboxy-terminus, respectively. In yet other embodiments, an exopeptidase in accordance with the disclosure is a tripeptidyl-peptidase, which cleaves a tripeptide from an amino-terminus. Peptidase classification and activities of each class or subclass thereof is well known and described in the literature (see, e.g., Gurupriya, V. S. & Roy, S. C. Proteases and Protease Inhibitors in Male Reproduction. Proteases in Physiology and Pathology 195-216 (2017); and Brix, K. & Stocker, W. Proteases: Structure and Function. Chapter 1). In some embodiments, a peptidase in accordance with the disclosure removes more than three amino acids from a polypeptide terminus. Accordingly, in some embodiments, the peptidase is an endopeptidase, e.g., that cleaves preferentially at particular positions (e.g., before or after a particular amino acid). In some embodiments, the size of a polypeptide cleavage product of endopeptidase activity will depend on the distribution of cleavage sites (e.g., amino acids) within the polypeptide being analyzed.

An exopeptidase in accordance with the disclosure may be selected or engineered based on the directionality of a sequencing reaction. For example, in embodiments of sequencing from an amino-terminus to a carboxy-terminus of a polypeptide, an exopeptidase comprises aminopeptidase activity. Conversely, in embodiments of sequencing from a carboxy-terminus to an amino-terminus of a polypeptide, an exopeptidase comprises carboxypeptidase activity. Examples of carboxypeptidases that recognize specific carboxy-terminal amino acids, which may be used as labeled exopeptidases or inactivated to be used as non-cleaving labeled recognition molecules described herein, have been described in the literature (see, e.g., Garcia-Guerrero, M. C., et al. (2018) PNAS 115(17)).

Suitable peptidases for use as cleaving reagents and/or recognition molecules include aminopeptidases that selectively bind one or more types of amino acids. In some embodiments, an aminopeptidase recognition molecule is modified to inactivate aminopeptidase activity. In some embodiments, an aminopeptidase cleaving reagent is non-specific such that it cleaves most or all types of amino acids from a terminal end of a polypeptide. In some embodiments, an aminopeptidase cleaving reagent is more efficient at cleaving one or more types of amino acids from a terminal end of a polypeptide as compared to other types of amino acids at the terminal end of the polypeptide. For example, an aminopeptidase in accordance with the disclosure specifically cleaves alanine, arginine, asparagine, aspartic acid, cysteine, glutamine, glutamic acid, glycine, histidine, isoleucine, leucine, lysine, methionine, phenylalanine, proline, selenocysteine, serine, threonine, tryptophan, tyrosine, and/or valine. In some embodiments, an aminopeptidase is a proline aminopeptidase. In some embodiments, an aminopeptidase is a proline iminopeptidase. In some embodiments, an aminopeptidase is a glutamate/aspartate-specific aminopeptidase. In some embodiments, an aminopeptidase is a methionine-specific aminopeptidase.

In some embodiments, an aminopeptidase is a non-specific aminopeptidase. In some embodiments, a non-specific aminopeptidase is a zinc metalloprotease.

Examples of cleaving reagents for use in accordance with the disclosure are described more fully in PCT International Application No. PCT/US2019/061831, filed Nov. 15, 2019, and PCT International Application No. PCT/US2021/033493, filed May 20, 2021, and a U.S. patent application entitled “Polypeptide Cleaving Reagents and Uses Thereof,” filed on even date herewith, the relevant contents of each of which are incorporated herein by reference in their entireties.

### Integrated Device & Instrument

Methods in accordance with the disclosure, in some aspects, may be performed using a system that permits single-molecule analysis. The system may include an integrated device and an instrument configured to interface with the integrated device. The integrated device may include an array of pixels, where individual pixels include a sample well and at least one photodetector. The sample wells of the integrated device may be formed on or through a surface of the integrated device and be configured to receive a sample placed on the surface of the integrated device. Collectively, the sample wells may be considered as an array of sample wells. The plurality of sample well may have a suitable size and shape such that at least a portion of the sample well receive a single sample (e.g., a single molecule, such as a polypeptide). In some embodiments, the number of samples within a sample well may be distributed among the sample wells of the integrated device such that some sample wells contain one sample while others contain zero, two or more samples.

Excitation light is provided to the integrated device from one or more light sources external to the integrated device. Optical components of the integrated device may receive the excitation light from the light source and direct the light towards the array of sample wells of the integrated device and illuminate an illumination region within the sample well. In some embodiments, a sample well may have a configuration that allows for the sample to be retained in proximity to a surface of the sample well, which may ease delivery of excitation light to the sample and detection of emission light from the sample. A sample positioned within the illumination region may emit emission light in response to being illuminated by the excitation light. For example, the sample may be labeled with a fluorescent label, which emits light in response to achieving an excited state through the illumination of excitation light. Emission light emitted by a sample may then be detected by one or more photodetectors within a pixel corresponding to the sample well with the sample being analyzed. When performed across the array of sample well, which may range in number between approximately 10,000 pixels to 1,000,000 pixels according to some embodiments, multiple samples can be analyzed in parallel.

The integrated device may include an optical system for receiving excitation light and directing the excitation light among the reaction chamber array. The optical system may include one or more grating couplers configured to couple excitation light to other optical components of the integrated device and direct the excitation light to the other optical components. For example, the optical system may include optical components that direct the excitation light from the grating coupler(s) towards the reaction chamber array. Such optical components may include optical splitters, optical combiners, and waveguides. In some embodiments, one or more optical splitters may couple excitation light from a grating coupler and deliver excitation light to at least one of the waveguides. According to some embodiments, the optical splitter may have a configuration that allows for delivery of excitation light to be substantially uniform across all the waveguides such that each of the waveguides receives a substantially similar amount of excitation light. Such embodiments may improve performance of the integrated device by improving the uniformity of excitation light received by sample wells of the integrated device. Examples of suitable components, e.g., for coupling excitation light to a reaction chamber and/or directing emission light to a photodetector, to include in an integrated device are described in U.S. patent application Ser. No. 14/821,688, filed Aug. 7, 2015, titled “INTEGRATED DEVICE FOR PROBING, DETECTING AND ANALYZING MOLECULES,” and U.S. patent application Ser. No. 14/543,865, filed Nov. 17, 2014, titled “INTEGRATED DEVICE WITH EXTERNAL LIGHT SOURCE FOR PROBING, DETECTING, AND ANALYZING MOLECULES,” both of which are incorporated by reference in their entirety. Examples of suitable grating couplers and waveguides that may be implemented in the integrated device are described in U.S. patent application Ser. No. 15/844,403, filed Dec. 15, 2017, titled “OPTICAL COUPLER AND WAVEGUIDE SYSTEM,” which is incorporated by reference in its entirety.

Additional photonic structures may be positioned between the sample wells and the photodetectors and configured to reduce or prevent excitation light from reaching the photodetectors, which may otherwise contribute to signal noise in detecting emission light. In some embodiments, metal layers which may act as a circuitry for the integrated device, may also act as a spatial filter. Examples of suitable photonic structures may include spectral filters, a polarization filters, and spatial filters and are described in U.S. patent application Ser. No. 16/042,968, filed Jul. 23, 2018, titled “OPTICAL REJECTION PHOTONIC STRUCTURES,” and U.S. Provisional Patent Application No. 63/124,655, filed Dec. 11, 2020, titled “INTEGRATED CIRCUIT WITH IMPROVED CHARGE TRANSFER EFFICIENCY AND ASSOCIATED TECHNIQUES,” both of which are incorporated by reference in their entirety.

Components located off of the integrated device may be used to position and align an excitation source to the integrated device. Such components may include optical components including lenses, mirrors, prisms, windows, apertures, attenuators, and/or optical fibers. Additional mechanical components may be included in the instrument to allow for control of one or more alignment components. Such mechanical components may include actuators, stepper motors, and/or knobs. Examples of suitable excitation sources and alignment mechanisms are described in U.S. patent application Ser. No. 15/161,088, filed May 20, 2016, titled “PULSED LASER AND SYSTEM,” which is incorporated by reference in its entirety. Another example of a beam-steering module is described in U.S. patent application Ser. No. 15/842,720, filed Dec. 14, 2017, titled “COMPACT BEAM SHAPING AND STEERING ASSEMBLY,” which is incorporated herein by reference. Additional examples of suitable excitation sources are described in U.S. patent application Ser. No. 14/821,688, filed Aug. 7, 2015, titled “INTEGRATED DEVICE FOR PROBING, DETECTING AND ANALYZING MOLECULES,” which is incorporated by reference in its entirety.

The photodetector(s) positioned with individual pixels of the integrated device may be configured and positioned to detect emission light from the pixel's corresponding reaction chamber. Examples of suitable photodetectors are described in U.S. patent application Ser. No. 14/821,656, filed Aug. 7, 2015, titled “INTEGRATED DEVICE FOR TEMPORAL BINNING OF RECEIVED PHOTONS,” which is incorporated by reference in its entirety. In some embodiments, a reaction chamber and its respective photodetector(s) may be aligned along a common axis. In this manner, the photodetector(s) may overlap with the reaction chamber within the pixel.

Characteristics of the detected emission light may provide an indication for identifying the label associated with the emission light. Such characteristics may include any suitable type of characteristic, including an arrival time of photons detected by a photodetector, an amount of photons accumulated over time by a photodetector, and/or a distribution of photons across two or more photodetectors. In some embodiments, such characteristics can be any one or a combination of two or more of luminescence lifetime, luminescence intensity, brightness, absorption spectra, emission spectra, luminescence quantum yield, wavelength (e.g., peak wavelength), and signal characteristics (e.g., pulse duration, interpulse durations, change in signal magnitude).

In some embodiments, a photodetector may have a configuration that allows for the detection of one or more timing characteristics associated with a sample's emission light (e.g., luminescence lifetime). The photodetector may detect a distribution of photon arrival times after a pulse of excitation light propagates through the integrated device, and the distribution of arrival times may provide an indication of a timing characteristic of the sample's emission light (e.g., a proxy for luminescence lifetime). In some embodiments, the one or more photodetectors provide an indication of the probability of emission light emitted by the label (e.g., luminescence intensity). In some embodiments, a plurality of photodetectors may be sized and arranged to capture a spatial distribution of the emission light. Output signals from the one or more photodetectors may then be used to distinguish a label from among a plurality of labels, where the plurality of labels may be used to identify a sample within the sample. In some embodiments, a sample may be excited by multiple excitation energies, and emission light and/or timing characteristics of the emission light emitted by the sample in response to the multiple excitation energies may distinguish a label from a plurality of labels.

In operation, parallel analyses of samples within the reaction chambers are carried out by exciting some or all of the samples within the chambers using excitation light and detecting signals from sample emission with the photodetectors. Emission light from a sample may be detected by a corresponding photodetector and converted to at least one electrical signal. The electrical signals may be transmitted along conducting lines in the circuitry of the integrated device, which may be connected to an instrument interfaced with the integrated device. The electrical signals may be subsequently processed and/or analyzed. Processing or analyzing of electrical signals may occur on a suitable computing device either located on or off the instrument.

The instrument may include a user interface for controlling operation of the instrument and/or the integrated device. The user interface may be configured to allow a user to input information into the instrument, such as commands and/or settings used to control the functioning of the instrument. In some embodiments, the user interface may include buttons, switches, dials, and a microphone for voice commands. The user interface may allow a user to receive feedback on the performance of the instrument and/or integrated device, such as proper alignment and/or information obtained by readout signals from the photodetectors on the integrated device. In some embodiments, the user interface may provide feedback using a speaker to provide audible feedback. In some embodiments, the user interface may include indicator lights and/or a display screen for providing visual feedback to a user.

In some embodiments, the instrument may include a computer interface configured to connect with a computing device. The computer interface may be a USB interface, a FireWire interface, or any other suitable computer interface. A computing device may be any general purpose computer, such as a laptop or desktop computer. In some embodiments, a computing device may be a server (e.g., cloud-based server) accessible over a wireless network via a suitable computer interface. The computer interface may facilitate communication of information between the instrument and the computing device. Input information for controlling and/or configuring the instrument may be provided to the computing device and transmitted to the instrument via the computer interface. Output information generated by the instrument may be received by the computing device via the computer interface. Output information may include feedback about performance of the instrument, performance of the integrated device, and/or data generated from the readout signals of the photodetector.

In some embodiments, the instrument may include a processing device configured to analyze data received from one or more photodetectors of the integrated device and/or transmit control signals to the excitation source(s). In some embodiments, the processing device may comprise a general purpose processor, a specially-adapted processor (e.g., a central processing unit (CPU) such as one or more microprocessor or microcontroller cores, a field-programmable gate array (FPGA), an application-specific integrated circuit (ASIC), a custom integrated circuit, a digital signal processor (DSP), or a combination thereof). In some embodiments, the processing of data from one or more photodetectors may be performed by both a processing device of the instrument and an external computing device. In other embodiments, an external computing device may be omitted and processing of data from one or more photodetectors may be performed solely by a processing device of the integrated device.

According to some embodiments, the instrument that is configured to analyze samples based on luminescence emission characteristics may detect differences in luminescence lifetimes and/or intensities between different luminescent molecules, and/or differences between lifetimes and/or intensities of the same luminescent molecules in different environments. The inventors have recognized and appreciated that differences in luminescence emission lifetimes can be used to discern between the presence or absence of different luminescent molecules and/or to discern between different environments or conditions to which a luminescent molecule is subjected. In some cases, discerning luminescent molecules based on lifetime (rather than emission wavelength, for example) can simplify aspects of the system. As an example, wavelength-discriminating optics (such as wavelength filters, dedicated detectors for each wavelength, dedicated pulsed optical sources at different wavelengths, and/or diffractive optics) may be reduced in number or eliminated when discerning luminescent molecules based on lifetime. In some cases, a single pulsed optical source operating at a single characteristic wavelength may be used to excite different luminescent molecules that emit within a same wavelength region of the optical spectrum but have measurably different lifetimes. An analytic system that uses a single pulsed optical source, rather than multiple sources operating at different wavelengths, to excite and discern different luminescent molecules emitting in a same wavelength region can be less complex to operate and maintain, more compact, and may be manufactured at lower cost.

Although analytic systems based on luminescence lifetime analysis may have certain benefits, the amount of information obtained by an analytic system and/or detection accuracy may be increased by allowing for additional detection techniques. For example, some embodiments of the systems may additionally be configured to discern one or more properties of a sample based on luminescence wavelength and/or luminescence intensity. In some implementations, luminescence intensity may be used additionally or alternatively to distinguish between different luminescent labels. For example, some luminescent labels may emit at significantly different intensities or have a significant difference in their probabilities of excitation (e.g., at least a difference of about 35%) even though their decay rates may be similar. By referencing binned signals to measured excitation light, it may be possible to distinguish different luminescent labels based on intensity levels.

According to some embodiments, different luminescence lifetimes may be distinguished with a photodetector that is configured to time-bin luminescence emission events following excitation of a luminescent label. The time binning may occur during a single charge-accumulation cycle for the photodetector. A charge-accumulation cycle is an interval between read-out events during which photo-generated carriers are accumulated in bins of the time-binning photodetector. Examples of a time-binning photodetector are described in U.S. patent application Ser. No. 14/821,656, filed Aug. 7, 2015, titled “INTEGRATED DEVICE FOR TEMPORAL BINNING OF RECEIVED PHOTONS,” which is incorporated herein by reference. In some embodiments, a time-binning photodetector may generate charge carriers in a photon absorption/carrier generation region and directly transfer charge carriers to a charge carrier storage bin in a charge carrier storage region. In such embodiments, the time-binning photodetector may not include a carrier travel/capture region. Such a time-binning photodetector may be referred to as a “direct binning pixel.” Examples of time-binning photodetectors, including direct binning pixels, are described in U.S. patent application Ser. No. 15/852,571, filed Dec. 22, 2017, titled “INTEGRATED PHOTODETECTOR WITH DIRECT BINNING PIXEL,” which is incorporated herein by reference.

In some embodiments, different numbers of fluorophores of the same type may be linked to different reagents in a sample, so that each reagent may be identified based on luminescence intensity. For example, two fluorophores may be linked to a first labeled recognition molecule and four or more fluorophores may be linked to a second labeled recognition molecule. Because of the different numbers of fluorophores, there may be different excitation and fluorophore emission probabilities associated with the different recognition molecules. For example, there may be more emission events for the second labeled recognition molecule during a signal accumulation interval, so that the apparent intensity of the bins is significantly higher than for the first labeled recognition molecule.

The inventors have recognized and appreciated that distinguishing biological or chemical samples based on fluorophore decay rates and/or fluorophore intensities may enable a simplification of the optical excitation and detection systems. For example, optical excitation may be performed with a single-wavelength source (e.g., a source producing one characteristic wavelength rather than multiple sources or a source operating at multiple different characteristic wavelengths). Additionally, wavelength discriminating optics and filters may not be needed in the detection system. Also, a single photodetector may be used for each reaction chamber to detect emission from different fluorophores. The phrase “characteristic wavelength” or “wavelength” is used to refer to a central or predominant wavelength within a limited bandwidth of radiation (e.g., a central or peak wavelength within a 20 nm bandwidth output by a pulsed optical source). In some cases, “characteristic wavelength” or “wavelength” may be used to refer to a peak wavelength within a total bandwidth of radiation output by a source.

According to an aspect of the present disclosure, an exemplary integrated device may be configured to perform single-molecule analysis in combination with an instrument as described above. It should be appreciated that the exemplary integrated device described herein is intended to be illustrative and that other integrated device configurations may be configured to perform any or all techniques described herein.

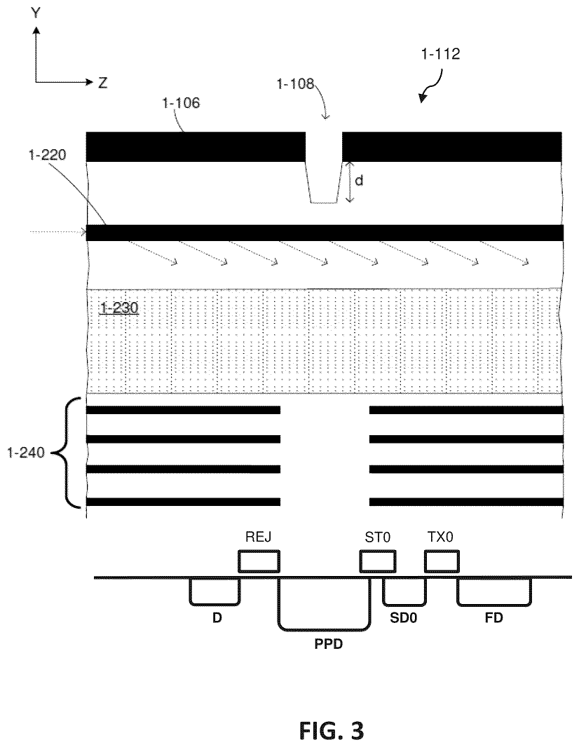

FIG. 3 illustrates a cross-sectional view of a pixel 1-112 of an integrated device 1-102. Pixel 1-112 includes a photodetection region, which may be a pinned photodiode (PPD), and a charge storage region, which may be a storage diode (SD0). In some embodiments, a photodetection region and charge storage regions may be formed in semiconductor material of a pixel by doping regions of the semiconductor material. For example, the photodetection region and charge storage regions can be formed using a same conductivity type (e.g., n-type doping or p-type doping).

During operation of pixel 1-112, excitation light may illuminate reaction chamber 1-108 causing incident photons, including fluorescence emissions from a sample, to flow along the optical axis to photodetection region PPD. As shown in FIG. 3, pixel 1-112 may include a waveguide 1 220 configured to optically (e.g., evanescently) couple excitation light from a grating coupler of the integrated device (not shown) to the reaction chamber 1-108. In response, a sample in the reaction chamber 1-108 may emit fluorescent light toward photodetection region PPD. In some embodiments, pixel 1-112 may also include one or more photonic structures 1-230, which may include one or more optical rejection structures such as a spectral filter, a polarization filter, and/or a spatial filter. For example, the photonic structures 1-230 may be configured to reduce the amount of excitation light that reaches the photodetection region PPD and/or increase the amount of fluorescent emissions that reach the photodetection region PPD. Also shown in pixel 1-112, pixel 1 112 may include one or more metal layers 1-240, which may be configured as a filter and/or may carry control signals from a control circuit configured to control transfer gates, as described further herein.

In some embodiments, pixel 1-112 may include one or more transfer gates configured to control operation of pixel 1-112 by applying an electrical bias to one or more semiconductor regions of pixel 1-112 in response to one or more control signals. For example, when transfer gate ST0 induces a first electrical bias at the semiconductor region between photodetection region PPD and storage region SD0, a transfer path (e.g., charge transfer channel) may be formed in the semiconductor region. Charge carriers (e.g., photo-electrons) generated in photodetection region PPD by the incident photons may flow along the transfer path to storage region SD0. In some embodiments, the first electrical bias may be applied during a collection period during which charge carriers from the sample are selectively directed to storage region SD0. Alternatively, when transfer gate ST0 provides a second electrical bias at the semiconductor region between photodetection region PPD and storage region SD0, charge carriers from photodetection region PPD may be blocked from reaching storage region SD0 along the transfer path. In some embodiments, drain gate REJ may provide a channel to drain D to draw noise charge carriers generated in photodetection region PPD by the excitation light away from photodetection region PPD and storage region SD0, such as during a rejection period before fluorescent emission photons from the sample reach photodetection region PPD. In some embodiments, during a readout period, transfer gate ST0 may provide the second electrical bias and transfer gate TX0 may provide an electrical bias to cause charge carriers stored in storage region SD0 to flow to the readout region, which may be a floating diffusion (FD) region, for processing.

It should be appreciated that, in accordance with various embodiments, transfer gates described herein may include semiconductor material(s) and/or metal, and may include a gate of a field effect transistor (FET), a base of a bipolar junction transistor (BJT), and/or the like.

In some embodiments, operation of pixel 1-112 may include one or more collection sequences, each collection sequence including one or more rejection (e.g., drain) periods and one or more collection periods. In one example, a collection sequence performed in accordance with one or more pulses of an excitation light source may begin with a rejection period, such as to discard charge carriers generated in pixel 1-112 (e.g., in photodetection region PD) responsive to excitation photons from the light source. For instance, the excitation photons may arrive at pixel 1-112 prior to the arrival of fluorescence emission photons from the reaction chamber. Transfer gates for the charge storage regions may be biased to have low conductivity in the charge transfer channels coupling the charge storage regions to the photodetection region, blocking transfer and accumulation of charge carriers in the charge storage regions. A drain gate for the drain region may be biased to have high conductivity in a drain channel between the photodetection region and the drain region, facilitating draining of charge carriers from the photodetection region to the drain region. Transfer gates for any charge storage regions coupled to the photodetection region may be biased to have low conductivity between the photodetection region and the charge storage regions, such that charge carriers are not transferred to or accumulated in the charge storage regions during the rejection period.

Following the rejection period, a collection period may occur in which charge carriers generated responsive to the incident photons are transferred to one or more charge storage regions. During the collection period, the incident photons may include fluorescent emission photons, resulting in accumulation of fluorescent emission charge carriers in the charge storage region(s). For instance, a transfer gate for one of the charge storage regions may be biased to have high conductivity between the photodetection region and the charge storage region, facilitating accumulation of charge carriers in the charge storage region. Any drain gates coupled to the photodetection region may be biased to have low conductivity between the photodetection region and the drain region such that charge carriers are not discarded during the collection period.

Some embodiments may include multiple rejection and/or collection periods in a collection sequence, such as a second rejection period and second collection period following a first rejection period and a collection period, where each pair of rejection and collection periods is conducted in response to a pulse of excitation light. In one example, charge carriers generated in the photodetection region during each collection period of a collection sequence (e.g., in response to a plurality of pulses of excitation light) may be aggregated in a single charge storage region. In some embodiments, charge carriers aggregated in the charge storage region may be read out for processing prior to the next collection sequence. Alternatively or additionally, in some embodiments, charge carriers aggregated in a first charge storage region during a first collection sequence may be transferred to a second charge storage region sequentially coupled to the first charge storage region and read out simultaneously with the next collection sequence. In some embodiments, a processing circuit configured to read out charge carriers from one or more pixels may be configured to determine one or more of luminescence intensity information, luminescence lifetime information, luminescence spectral information, and/or any other mode of luminescence information associated with performing techniques described herein.

In some embodiments, a first collection sequence may include transferring, to a charge storage region at a first time following each excitation pulse, charge carriers generated in the photodetection response in response to the excitation pulse, and a second collection sequence may include transferring, to the charge storage region at a second time following each excitation pulse, charge carriers generated in the photodetection response in response to the excitation pulse. For example, the number of charge carriers aggregated after the first and second times may indicate luminance lifetime information of the received light.

As described further herein, pixels of an integrated device may be controlled to perform one or more collection sequences using one or more control signals from a control circuit of the integrated circuit, such as by providing the control signal(s) to drain and/or transfer gates of the pixel(s) of the integrated circuit. In some embodiments, charge carriers may be read out from the FD region of each pixel during a readout pixel associated with each pixel and/or a row or column of pixels for processing. In some embodiments, FD regions of the pixels may be read out using correlated double sampling (CDS) techniques.

### Example 1

In this Example, a phospho-ubiquitin protein comprising a phosphoserine (pSer) at position 65 (pS65-Ub) was characterized.

Ubiquitin (Ub) is a small, 76-residue signaling protein that is specifically phosphorylated at serine 65 (S65) by PTEN-induced kinase 1 (PINK1). The level of phosphorylated Ub increases significantly under conditions of neurodegeneration, oxidative stress, or aging. However, under normal conditions, only a fraction of Ub is phosphorylated.

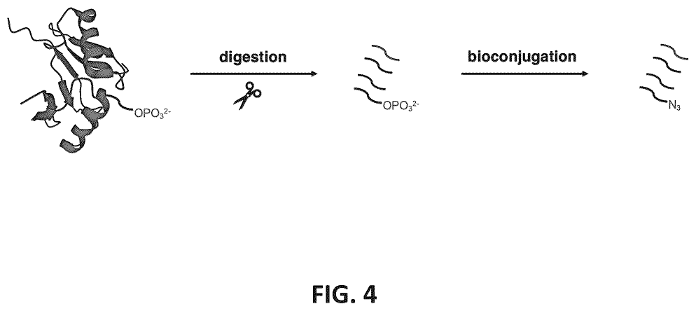

In this Example, pS65-Ub was subjected to a biocompatible two-step reaction. As shown in FIG. 4, the pSer residue of pS65-Ub was selectively functionalized with an azide click chemistry handle for immobilization on semiconductor chips (i.e., integrated devices). As further shown in FIG. 4, the selectively functionalized protein was then digested to yield a plurality of peptide fragments. In particular, the selectively functionalized protein was digested with Arg-C, an endopeptidase that cleaves at the C-terminus of arginine residues, yielding 4 peptide fragments, and the entire mixture was incubated with a macromolecular linker. This sample was subjected to a de salting column and utilized for on-chip sequencing using Quantum-Si's Platinum™ instrument.

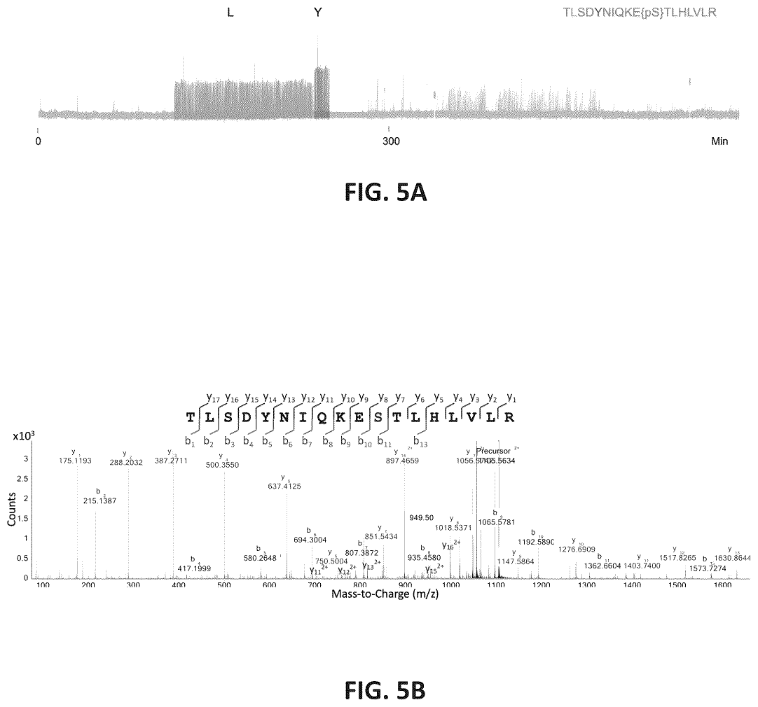

A sequencing reaction was performed on the peptide fragments using a combination of two amino acid recognition molecules—P5610 (F, Y, W) and PS1223 (L, I, V)—and two aminopeptidases. FIG. 5A shows a representative trace, which shows that the sequencing reaction began at the N-terminus and stopped at the site of pSer conjugation. The aminopeptidases cleaved each N-terminal amino acid (NAA), sequentially exposing the next amino acid in the peptide sequence for recognition. The first recognition segment (RS)—a period between aminopeptidase cleavage and NAA recognizer binding on/off—was the leucine (L) amino acid, which was recognized by PS1223 and was automatically highlighted by Quantum-Si's cloud software in orange color. The next RS in the sequence was the tyrosine (Y) amino acid, which was recognized by PS610 and highlighted in blue color. The sequencing reaction terminated when the aminopeptidases stopped cutting at the site where the pSer residue was conjugated. The presence of L and Y in the trace demonstrated that the location of the pSer residue was at the second serine residue of the peptide fragment.

To validate the results, Arg-C digested pS65-Ubiquitin was analyzed using a platform comprised of an Agilent 1290 Infinity II UPLC system coupled to an Agilent 6550 iFunnel Q-TOF. Data acquired using an auto MS/MS is shown in FIG. 5B, showing phosphorylation of S65 of the peptide and confirming the sequencing results obtained from Quantum-Si's Platinum T^{m} instrument.

Thus, this Example demonstrates the ability to directly label and sequence pSer proteins to provide unambiguous detection of phosphoproteins.

### Experimental Protocol

pS65-Ubiquitin (150 μg, 8.6 kDa protein) was supplied at a concentration of 250 μM (U 102-150_24069218D; R&D Systems) in 10 mM HEPES pH 7.5. This protein was digested using Arg-C sequencing grade protease (Promega, V188 Å) and the resulting peptide fragments were subjected to dehydroalanine (Dha) formation by reaction with barium hydroxide Ba(OH)₂. The peptide fragments were then subjected to Michael addition with azido-PEG₃-thioacetate. Thus, the pSer group at position 65 was converted to an azide.

This 250 μM sample (10 μL) was diluted 10-fold in 90 μL of incubation buffer (50 mM Tris-HCl pH 7.5, 5 mM CaCl₂), and 2 mM EDTA), generating 25 μM pS65-Ubiquitin. The incubation buffer comprised 50 mM Tris-HCl, pH 7.5, 5 mM CaCl₂), and 2 mM EDTA.

Arg-C sequencing grade protease (Promega, V188A, 10 μg) was resuspended in 20 μL of incubation buffer, generating a 0.5 μg/μL Arg-C protease stock. 2 μL of 0.5 μg/μL Arg-C stock was added to the pS65-Ubiquitin sample. Then 11 μL of 10× activation buffer was added to the pS65-Ubiquitin sample. The 10× activation buffer comprised 50 mM Tris-HCl, pH 7.5, 50 mM DTT, and 2 mM EDTA. The microcentrifuge tube was briefly centrifuged to collect the components at the bottom of the tube and incubated for 3 hours at 37° C. After digestion, 50 μL of the sample was removed and the reaction was quenched with 2.5 μL of 10% trifluoroacetic acid (TFA) for a final concentration of 0.5% TFA.

To a 1.5 mL microcentrifuge tube was added 20 μL of a 25 μM solution of digested pS65-Ubiquitin, followed by 5 μL of a 1.68 mM Ba(OH)₂ solution. The tube was mixed by vortexing and briefly spun down in a tabletop microcentrifuge. The tube was incubated at 37° C. for 30 min. Then, 1 μL of a 500 μM azido-PEG₃-thioacetate was added and the tube was placed back at 37° C. for 24 h. The reaction was quenched by addition of 2 μL of a 5 mM AcOH solution. The peptide solution was taken to click reaction with DBCO-Q24D-Cy3B complex.

General procedure for clicking azide-containing polypeptides with DBCO-Q24D-Cy3B complex: 3.4 μL of a 30 μM DBCO-Q24D-Cy3B complex, 1 μL of a 0.8-1 mM peptide solution, and 16.1 μL 1×PBS were added to a 1.5 mL microcentrifuge tube. The tube was mixed by vortexing and briefly spun down in a tabletop microcentrifuge. The tube was incubated at 37° C. for 12 hours. Then, excess peptides were removed using Zeba 7K MWCO spin filter (75 μL capacity). Briefly, the spin filter was placed in a microcentrifuge for 1 min at 3,200 rpm, followed by 20 μL peptide storage buffer (60 mM KOAc, 50 mM MOPS, pH 8.0) to the filter, spun at 3,200 rpm for 1 min. The used collection tube and liquid were discarded and the collection tube was changed, followed by loading the reaction mixture to the filter. The filter was spun at 3,200 rpm for 1 min. The filtrate contained the pure peptide-Q24D-SV-Cy3B complex. The absorbance at 564 nm was obtained, and the concentration was calculated using 130,000 M⁻¹ cm¹ extinction coefficient. This sample was subjected to de-salting column and utilized for on-chip sequencing.

### Example 2

In this Example, samples comprising a phosphoserine-containing portion of p53 protein, both alone and in mixture with other peptides, were characterized.

Purification and enrichment of phosphorylated proteins typically rely on affinity chromatography or selective precipitation methods. These methods are often lengthy and may result in loss of material. Instead of requiring additional experimental protocols for enriching phosphorylated proteins, Quantum-Si's semiconductor chips can facilitate enriching phosphorylated peptides from a mixture during the loading process. In this Example, samples comprising p53 alone and in mixture with up to 10 non-phosphorylated peptides were successfully characterized.

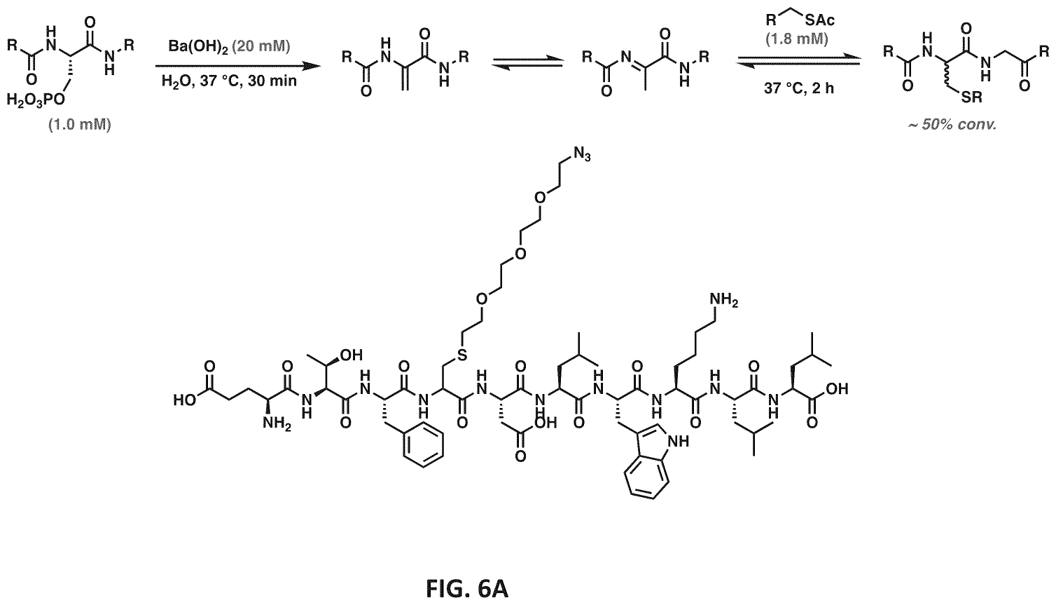

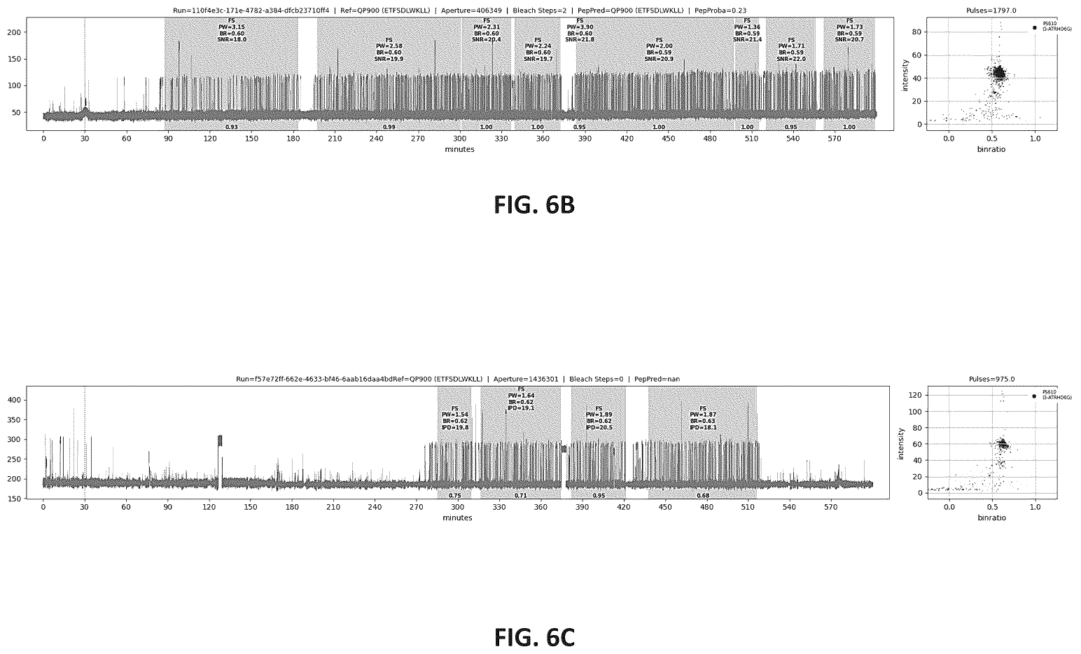

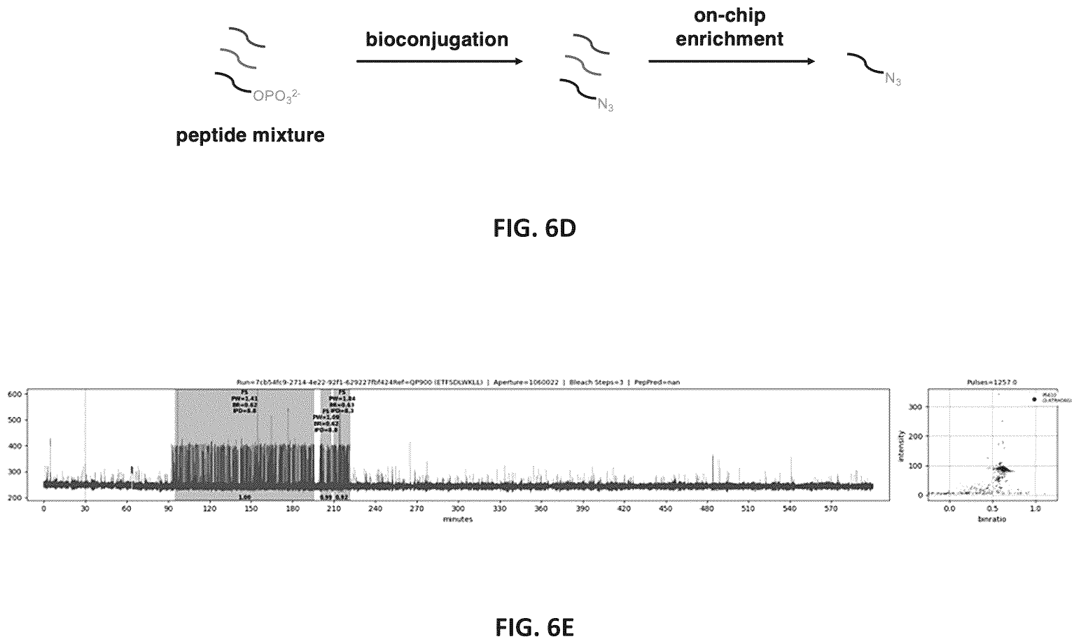

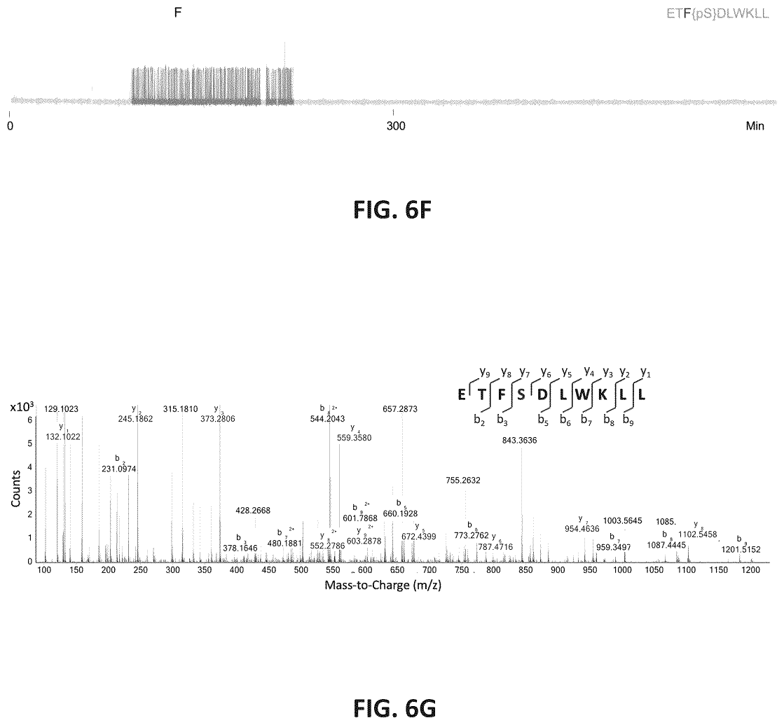

First, samples of p35 alone were characterized. A phosphoserine-containing fragment of p53 protein was selectively functionalized with an azide click chemistry handle according to the scheme shown in FIG. 6A. The functionalized protein fragment was then coupled to a macromolecular linker and utilized for on-chip sequencing using Quantum-Si's Platinum™ instrument. Representative traces are shown in FIGS. 6B and 6C. These traces confirm that Quantum-Si's Platinum™ instrument identified the phenylalanine (F) residue immediately preceding the phosphoserine residue. Sequencing terminated upon reaching the phosphoserine residue. This demonstrated that the serine residue of the peptide fragment was phosphorylated.

Second, a sample of p53 in a 1:1 mixture with a peptide having the sequence EFIAWLVAYPDDDWK-OH (SEQ ID NO: 10) (QP1042) was characterized. The p53 fragment was selectively functionalized as described above, and the functionalized p53 fragment was mixed in a 1:1 mixture with QP1042. During the on-chip loading and rinsing process, only the p53 peptide was immobilized to the bottom of the semiconductor chip, and the QP1042 peptide was rinsed off. A representative trace is shown in FIG. 6D. From FIG. 6D, it can be seen that the phenylalanine (F) residue immediately preceding the phosphoserine residue was again identified.

Third, a sample of p53 mixed with 10 different peptides (shown in Table 3) was characterized. During the on-chip loading and rinsing process, only the p53 peptide was immobilized at the bottom of the semiconductor chip, and all non-phosphorylated peptides were rinsed off. FIGS. 6E-6F highlight the results obtained from Quantum-Si's Platinum™ and comparison data from a QTOF instrument. As shown in FIG. 6E, only the p53 peptide comprising a phosphoserine residue was immobilized at the bottom of the semiconductor. Sequencing began at the N-terminus and stopped at the phosphoserine residue location. QTOF data shown in FIG. 6F confirms the site of phosphorylation.

TABLE 3

| Peptide sequence (N-Term→C-Term) | SEQ ID NO: | # Amino Acids | Mass (M + H) |
|---|---|---|---|
| H-ALYKKLLKKLLKSAKKLG-OH | 11 | 18 | 2043.8 |
| Myristoyl-RLYRKRIWRSAGR-OH | 12 | 13 | 1928.5 |
| H-LDYVNRRKMYQ-OH | 13 | 11 | 1486.1 |
| H-EMRISRIILDFLFLRKK-OH | 14 | 17 | 2178.8 |
| H-RIIDLLWRVRRPQKPKFVTVWVR-OH | 15 | 23 | 2962.8 |
| H-ASVYAWNRKR-OH | 16 | 10 | 1250.5 |
| H-FVQWFSKFLGRIL-NH2 | 17 | 13 | 1640.1 |
| H-WWGKKYRASKLGLAR-OH | 18 | 15 | 1820.3 |
| H-TFLLRNPNDK-NH2 | 19 | 10 | 1216.5 |
| H-ILRGSVAHK-OH | 20 | 9 | 980.2 |

Thus, this Example demonstrates the ability to selectively enrich phosphoserine-containing peptides to provide unambiguous detection of phosphoproteins.

### Experimental Protocols

p53

To a 1.5 mL microcentrifuge tube was added 3.7 μL of a 5.4 mM p53 peptide solution, followed by 1.8 μL of a 220 mM saturated Ba(OH)₂ solution and 14.5 μL of a 1 M NaOH. The tube was mixed by vortexing and briefly spun down in a tabletop microcentrifuge. The tube was incubated at 37° C. for 30 min. Then, 1.5 μL of a 25 mM azido-PEG₃-thioacetate and 3.5 μL of a 1 M NaOH were added and the tube was placed back at 37° C. for 2 h. The reaction was quenched by addition of 1 μL of a 600 mM AcOH solution. The peptide solution was then conjugated to a DBCO-Q24D-Cy3B complex, as described in Example 1.

p53+10 different peptides

To a 1.5 mL microcentrifuge tube were added 10 different peptides, 1 μL of a 1 mM of each peptide solution. To this tube was then added 1 μL of a 5.4 mM p53 peptide solution, followed by 2.2 μL of a 50 mM Ba(OH)₂ and 1.8 μL of a 1 M NaOH solutions. The tube was mixed by vortexing and briefly spun down in a tabletop microcentrifuge. The tube was incubated at 37° C. for 30 min. Then, 2.0 μL of a 5 mM azido-PEG₃-thioacetate was added and the tube was placed back at 37° C. for 2 h. The reaction was quenched by addition of 2.5 μL of a 50 mM AcOH solution. The peptide solution was then conjugated to a DBCO-Q24D-Cy3B complex, as described in Example 1.

### Example 3

In this Example, TIF1β, a member of the TIF1 subfamily of chromatin-associated transcriptional intermediary factors (TIFs) that regulate gene expression and T cell activation, was characterized. TIF1β is phosphorylated at two serine residues: Ser473 and Ser824.

In this Example, the phosphoserine residues of TIF1β, pSer473 and pSer824, were selectively functionalized with azide groups. Synthetic peptides corresponding to a Lys-C digest of TIF1β protein containing pSer473 and pSer824 were generated.

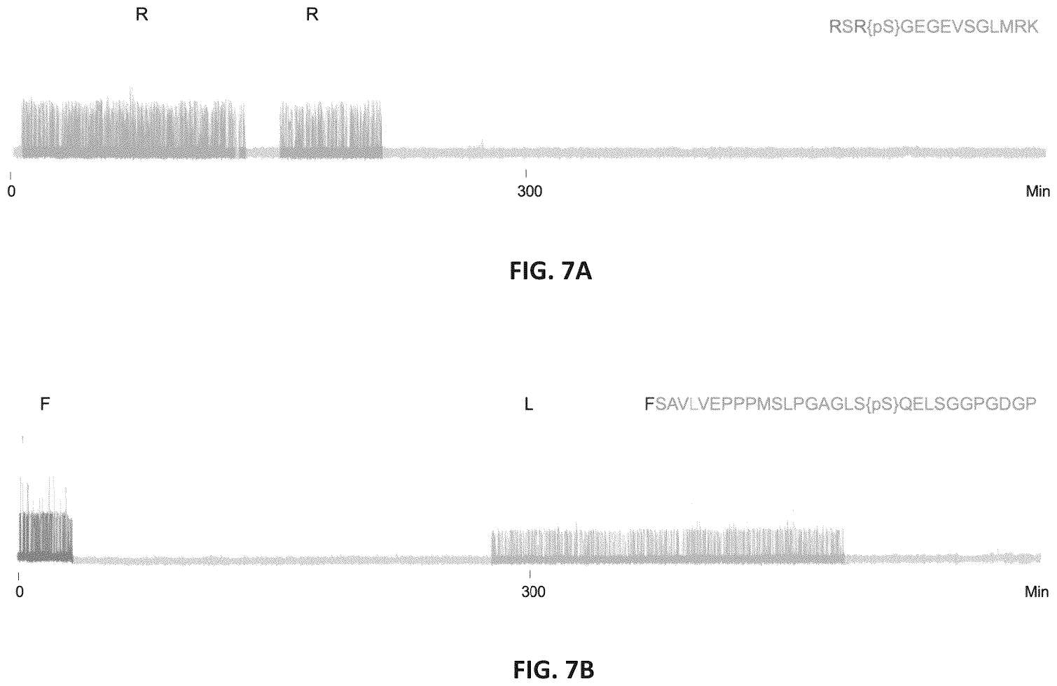

A representative traces of the first fragment, which had the sequence RSR{pS}GEGEVSGLMRK (SEQ ID NO: 21), is shown in FIG. 7A. From FIG. 7A, it can be seen that two arginine (R) residues were identified prior to termination upon reaching the phosphoserine residue. The presence of the two R residues in the trace demonstrated that the location of the pSer residue was at the second serine residue of the peptide fragment.

A representative trace of the second fragment, which had the sequence FSAVLVEPPPMSLPGAGLS{pS}QELSGGPGDGP (SEQ ID NO: 22), is shown in FIG. 7B. From FIG. 7B, it can be seen that a phenylalanine (F) residue and a leucine (L) residue were identified prior to termination upon reaching the proline residue.

### Example 4

In this Example, cardiac phospholamban (PLB), phosphorylation of which is responsible for intracellular Ca²⁺ concentration regulation and affects myocardial physiology and pathology, was characterized. PLB is phosphorylated at Ser16, producing phosphorylated cardiac phospholamban (pPLB).

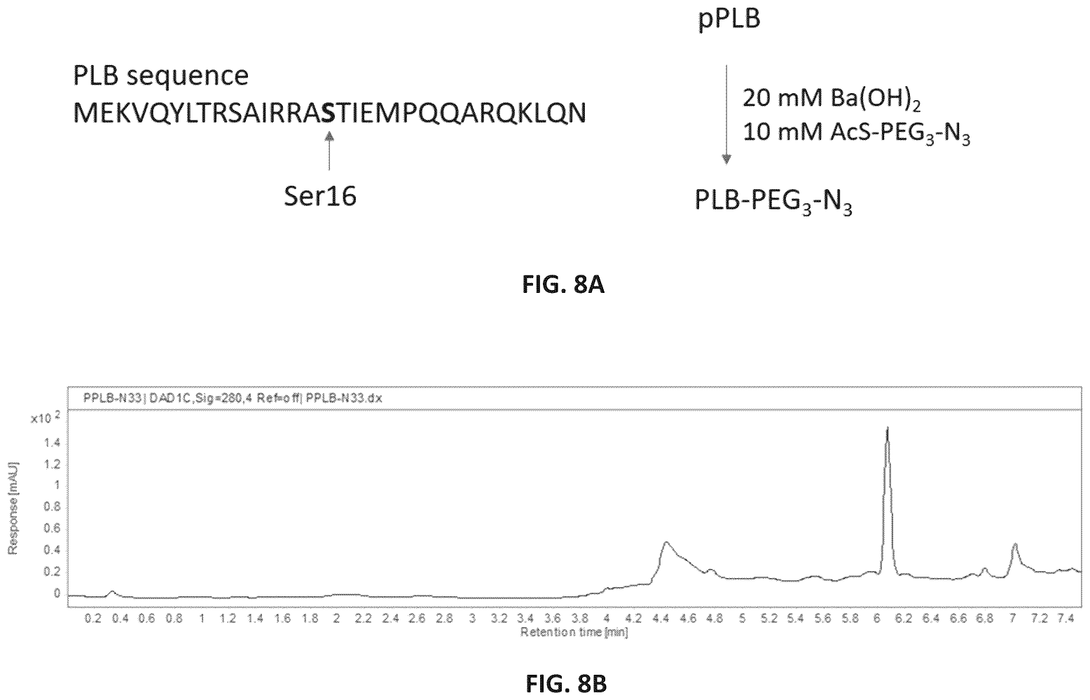

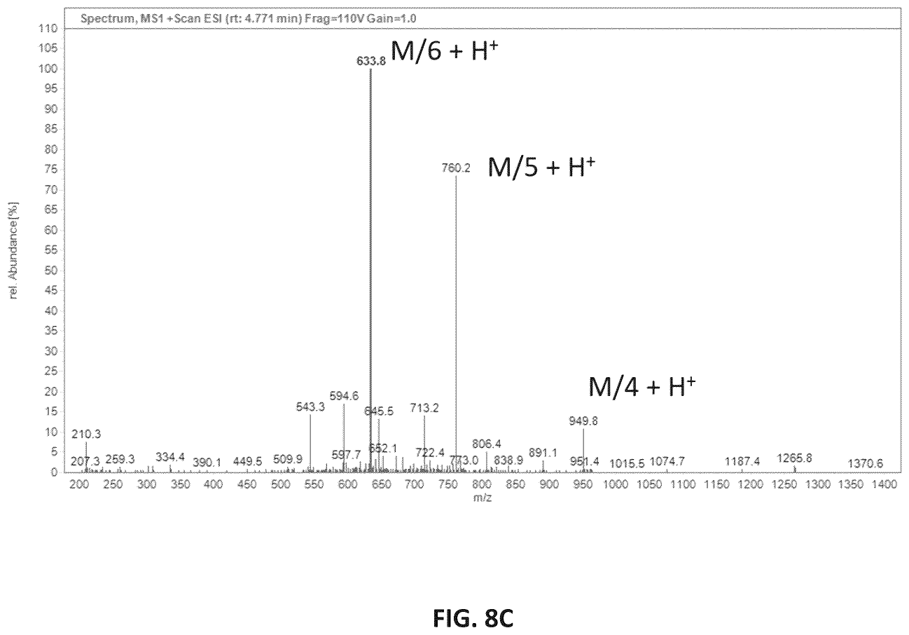

The sequence of PLB and the selective functionalization of the phosphoserine residue of pPLB with an azide group are shown in FIG. 8A. As shown in the LC-MS data in FIGS. 8B-8C, the phosphoserine residue of pPLB was functionalized with an azide group.

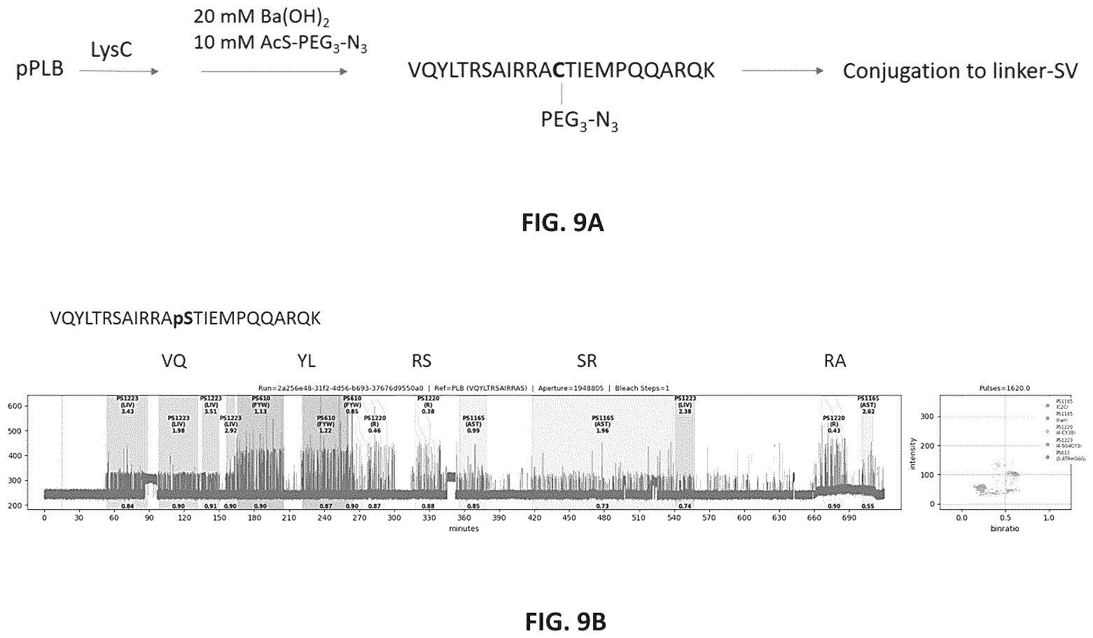

In this Example, pPLB was subjected to a biocompatible two-step reaction. As shown in FIG. 9A, pPLB was digested with Lys-C, an endopeptidase that cleaves at the C-terminus of lysine residues, to yield a plurality of peptide fragments. The peptide fragments were then subjected to conditions that selectively functionalized the phosphoserine residue with an azide group, and the mixture was incubated with linker-SV. A representative trace of the fragment, which had the sequence VQYLTRSAIRRA{pS}TIEMPQQARQK (SEQ ID NO: 23), is shown in FIG. 9B.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the application describes “a composition comprising A and B,” the application also contemplates the alternative embodiments “a composition consisting of A and B” and “a composition consisting essentially of A and B.”

Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof.

1. A method, comprising:
reacting a polypeptide comprising at least one dehydroalanine residue with a protected thiol compound of Formula (I):
R₁-(L₁)ₘ-S—X (I),
or a salt thereof, to provide a functionalized polypeptide comprising a residue of Formula (II): 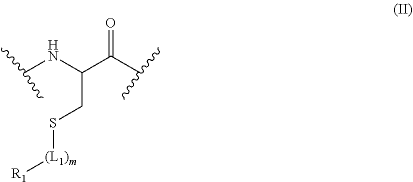
wherein:
X is a protecting group;
L₁ is a linking group;
m is an integer from 0 to 36; and
R₁ is a moiety comprising a click chemistry handle.

2. The method of claim 1, wherein X is selected from the group consisting of acetyl, allyl, propargyl, succinimide, thioether, thioester, glycosyl, alkenyl, diphenylmethyl, tetrahydropyranyl, 9-fluorenylmethyl, 9H-xanthen-9-yl, pseudoproline, tert-butyl, urea, aryl, benzyl, trityl, and disulfide.

3. The method of claim 1, wherein L₁ is selected from the group consisting of [symbol] substituted or unsubstituted alkylene, substituted or unsubstituted alkenylene, substituted or unsubstituted alkynylene, substituted or unsubstituted heteroalkylene, substituted or unsubstituted heteroalkenylene, substituted or unsubstituted heteroalkynylene, substituted or unsubstituted carbocyclylene, substituted or unsubstituted heterocyclylene, substituted or unsubstituted arylene, substituted or unsubstituted heteroarylene, or a combination thereof.

4. The method of claim 1, wherein L₁ comprises a substituted or unsubstituted heteroalkylene.

5. The method of claim 4, wherein the heteroalkylene comprises an alkylene oxide.

6. The method of claim 4, wherein the alkylene oxide comprises polyethylene glycol.

7. The method of claim 1, wherein m is an integer from 1 to 10.

8. The method of claim 1, wherein m is an integer from 1 to 5.

9. The method of claim 1, wherein m is 0.

10. The method of claim 1, wherein the click chemistry handle comprises an azide, tetrazine, nitrile oxide, alkyne, or alkene.

11. (canceled)

12. The method of claim 1, wherein a molar ratio of R₁-(L)ₘ-S—X to the polypeptide is in a range from 1.5:1 to 10:1.

13. The method of claim 1, wherein the polypeptide has 2-100 amino acid residues.

14. A method, comprising:
converting a phosphoserine residue of a polypeptide to a dehydroalanine residue to provide a dehydroalanine-containing polypeptide;
reacting the dehydroalanine-containing polypeptide with a protected thiol compound of Formula (I):
R₁-(L₁)ₘ-S—X (I),
or a salt thereof, to provide a functionalized polypeptide comprising a residue of Formula (II): 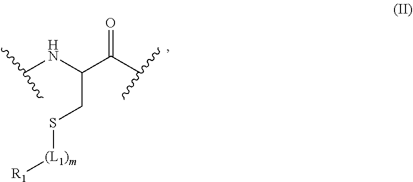
wherein:
X is a protecting group;
L₁ is a linking group;
m is an integer from 0 to 36; and
R₁ is a moiety comprising a click chemistry handle;
digesting the functionalized polypeptide to form two or more peptide fragments,
wherein at least one peptide fragment is a functionalized peptide fragment comprising R₁; and
conjugating the functionalized peptide fragment to a linker.

15. A method, comprising:
digesting a polypeptide comprising a phosphoserine residue to form two or more peptide fragments, wherein at least one peptide fragment comprises the phosphoserine residue;
converting the phosphoserine residue of the at least one peptide fragment to a dehydroalanine residue of the at least one peptide fragment to provide a dehydro alanine-containing peptide fragment;
reacting the dehydroalanine-containing peptide fragment with a protected thiol compound of Formula (I):
R₁-(L₁)ₘ-S—X (I),
or a salt thereof, to provide a functionalized peptide fragment comprising a residue of Formula (II): 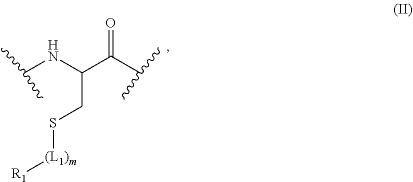
wherein:
X is a protecting group;
L₁ is a linking group;
m is an integer from 0 to 36; and
R₁ is a moiety comprising a click chemistry handle; and
conjugating the functionalized peptide fragment to a linker.

16-54. (canceled)

55. A kit, comprising:
a protected thiol compound comprising a click chemistry handle;
a base; and
a linker.

56-89. (canceled)

90. A method of enriching a sample comprising one or more polypeptides comprising a target residue, comprising:
selectively conjugating the target residue to a linker;
immobilizing the linker to a surface; and
removing any non-immobilized polypeptides or portions of polypeptides.

91-101. (canceled)

102. A system, comprising:
an integrated device comprising a sample well; and
a peptide immobilized to a surface of the sample well at a target residue, wherein the target residue is not a terminal amino acid of the peptide.

103-110. (canceled)

111. A method of characterizing a peptide, comprising:
immobilizing the peptide to a surface of a sample well at a target residue, wherein the target residue is not a terminal amino acid of the peptide;
contacting the peptide with one or more amino acid recognition molecules labeled with one or more detectable labels;
detecting one or more series of signal pulses indicative of binding events between the one or more amino acid recognition molecules and the peptide; and
determining at least one chemical characteristic of the peptide.

112-121. (canceled)

122. A method of characterizing a phosphoserine residue of a polypeptide, comprising:
(a) converting the phosphoserine residue of the polypeptide to a dehydroalanine residue to provide a dehydroalanine-containing polypeptide;
(b) reacting the dehydroalanine-containing polypeptide with a thiol compound comprising a click chemistry handle to provide a functionalized polypeptide comprising a functionalized dehydroalanine residue;
(c) digesting the functionalized polypeptide to form two or more peptide fragments, wherein at least one peptide fragment is a functionalized peptide fragment comprising a functionalized dehydroalanine residue;
(d) contacting the functionalized peptide fragment with one or more amino acid recognition molecules;
(e) detecting a first series of signal pulses indicative of binding events between the one or more amino acid recognition molecules and a first amino acid at a terminus of the functionalized peptide fragment;
(f) determining at least one chemical characteristic of an amino acid of the functionalized peptide fragment based on at least one characteristic of the first series of signal pulses;
(g) removing the first amino acid from the terminus of the functionalized peptide fragment; and
(h) repeating steps (d) to (g) until the functionalized dehydroalanine residue is exposed at the terminus of the functionalized peptide fragment.

123. A method of characterizing a phosphoserine residue of a polypeptide, comprising:
(a) digesting the polypeptide to form two or more peptide fragments, wherein at least one peptide fragment is a peptide fragment comprising the phosphoserine residue;
(b) converting the phosphoserine residue of the at least one peptide fragment to a dehydroalanine residue of the at least one peptide fragment to provide a dehydro alanine-containing peptide fragment;
(c) reacting the dehydroalanine-containing peptide fragment with a thiol compound comprising a click chemistry handle to provide a functionalized peptide fragment comprising a functionalized dehydroalanine residue;
(d) contacting the functionalized peptide fragment with one or more amino acid recognition molecules;
(e) detecting a first series of signal pulses indicative of binding events between the one or more amino acid recognition molecules and a first amino acid at a terminus of the functionalized peptide fragment;
(f) determining at least one chemical characteristic of an amino acid of the functionalized peptide fragment based on at least one characteristic of the first series of signal pulses;
(g) removing the first amino acid from the terminus of the functionalized peptide fragment; and
(h) repeating steps (d) to (g) until the functionalized dehydroalanine residue is exposed at the terminus of the functionalized peptide fragment.

124-135. (canceled)
